SEQUENCE LISTING (256 sequences)

SEQ ID NO: 1 - DNA, 128 bp, Artificial Sequence
  Fusion sequence
ctcctgctcctcgtcgttgctgtcttctactgcgtgagacgcaaagggggcccctgctgccgccagcggcgggagaagggggctccggaggagtggatgcgggccatccagatggtcgccaacagcct

SEQ ID NO: 2 - DNA, 135 bp, Artificial Sequence
  Fusion Sequence
gacgacgtagcagccatcttttccctggctttggtgattcagccctgacttctcaaaaagcactgcacagaggaggaggcagcagaaccccatggaggagtggatgcgggccatccagatggtcgccaacagcct

SEQ ID NO: 3 - DNA, 150 bp, Artificial Sequence
  Fusion sequence
agtctgggccacacgatttgacggaaacatcttacttgccaaggcaagatctagatgctcgtgtacaagtttgccagaaaacaccacatcactttaactaatctaattactgaagagactactcatgttgttatgaaaacagatgctgag

SEQ ID NO: 4 - DNA, 102 bp, Artificial Sequence
  Fusion sequence
gggtgacccagtctattaaagaaagaaaaatgctgaatgagggtgtccacccaattgtggttgtgcagccagatgcctggacagaggacaatggcttccatg

SEQ ID NO: 5 - DNA, 168 bp, Artificial Sequence
  Fusion sequence
cctggaagtaattgtaagcatcctgaaataaaaaagcaagaatatgaagaagtagttcagactgttaatacagatttctctccatatctgatttcagataacttagaacagcctatgggaatattaacttcacagaaaagtagtgaataccctataagccagaatcca

SEQ ID NO: 6 - DNA, 117 bp, Artificial Sequence
  Fusion sequence
ccttcacagtgtcctttatgtaagaatgatataaccaaaaggtcatccccttctaaatgcccatcattagatgataggtggtacatgcacagttgctctgggagtcttcagaataga

SEQ ID NO: 7 - DNA, 136 bp, Artificial Sequence
  Fusion sequence
gaactgtgagaactctgaggacaaagcagcggatacaacctcaaaagacgtctgtctacattgaattggcagagggataccatgcaacataacctgataaagctccagcaggaaatggctgaactagaagctgtgt

SEQ ID NO: 8 - DNA, 116 bp, Artificial Sequence
  Fusion sequence
gaactgtgagaactctgaggacaaagcagcggatacaacctcaaaagacgtctgtctacattgaattggtattaacttcacagaaaagtagtgaataccctataagccagaatcca

SEQ ID NO: 9 - DNA, 115 bp, Artificial Sequence
  Fusion sequence
agtctgggccacacgatttgacggaaacatcttacttgccaaggcaagatctagatgctgagtttgtgtgtgaacggacactgaaatattttctaggaattgcgggaggaaaatg

SEQ ID NO: 10 - DNA, 136 bp, Artificial Sequence
  Fusion sequence
gaagtcagaggagatgtggtcaatggaagaaaccaccaaggtccaaagcgagcaagagaatcccaggacagaaagggtgtccacccaattgtggttgtgcagccagatgcctggacagaggacaatggcttccatg

SEQ ID NO: 11 - DNA, 149 bp, Artificial Sequence
  Fusion sequence
tgcatcatgtttctttagagccgattacctgtgtaccctttcgggctctgtgtgacactccaggtgtggatccaaagcttatttctagaatttgggtttataatcactatagatggatcatatggaaactggcagctatggaatgtgcc

SEQ ID NO: 12 - DNA, 135 bp, Artificial Sequence
  Fusion sequence
gtcagcttactccggccaaaaaagaactgcacctctggagcggatttaggaccaataagtcttaattggtttgaagaactttcttcagaagctccaccctataattctgaacctgcagaagaatctgaacataaa

SEQ ID NO: 13 - DNA, 149 bp, Artificial Sequence
  Fusion sequence
ccatcacgtgcactaacaagacagcaagttcgtgctttgcaagatggtgcagagctttatgaagcagtgaagaatgcagcagacccagcttaccttgaggacttgcccctttcgtctatttgtcagacgaatgttacaatttactggca

SEQ ID NO: 14 - DNA, 150 bp, Artificial Sequence
  Fusion sequence
ttctgaaagtctaggagctgaggtggatcctgatatgtcttggtcaagttctttagctacaccacccacccttagttctactgtgctcataggatttggaaaaacatcagggaattcatttaaagtaaatagctgcaaagaccacattgg

SEQ ID NO: 15 - DNA, 161 bp, Artificial Sequence
  Fusion sequence
acgaggcattggatgattcagaggatattcttcataactctctagataatgatgaatgtagcacgcattcacattccttacacaaagttaagggagtgttagaggaatttgatttaatcagaactgagcatagtcttcactattcacctacgtctagacaa

SEQ ID NO: 16 - DNA, 110 bp, Artificial Sequence
  Fusion sequence
gcatgtctaacagctattcctaccattctgatgaggtatataatgattcaggatatggtttatcaagggatgtcacaaccgtgtggaagttgcgtattgtaagctattca

SEQ ID NO: 17 - DNA, 133 bp, Artificial Sequence
  Fusion sequence
acttgattctggtattgagccagtattgaagaatgttgaagatcaaagtcctttatcactttgtatggccaaaaggaagtctgtttccacacctgtctcagcccagatgacttcaaagtcttgtaaaggggag

SEQ ID NO: 18 - DNA, 143 bp, Artificial Sequence
  Fusion sequence
ccagcttcctataacttggacgatgaccaggcggcttgggagaatgagctgcagaagatgacccgggggcaggaggatccaaagtgggaattccctcggaagaacttggttcttggaaaaactctaggagaaggcgaatttgg

SEQ ID NO: 19 - DNA, 145 bp, Artificial Sequence
  Fusion sequence
ggcttaagacactagagattgctttggagcagaagaaggaggagtgtctgaaaatggaatcacaattgaaaaaggaggatccaaagtgggaattccctcggaagaacttggttcttggaaaaactctaggagaaggcgaatttgg

SEQ ID NO: 20 - DNA, 133 bp, Artificial Sequence
  Fusion sequence
caggcagaagttgatcgactcttagaaatcttgaaggaggtggaaaatgagaagaatgacaaagataagaagatagctgagttggaaagtactcttccaacccaagaggagattgaaaatcttcctgccttcc

SEQ ID NO: 21 - DNA, 143 bp, Artificial Sequence
  Fusion sequence
gcagtctctggcagaaaaggaaactcacttgactaatcttcgggcagagagaaggaaacacttagaggaagttctggagatgaagtgtccacgtgagctgccgcccacgctgctggggaacagttccgaagaggagagccagc

SEQ ID NO: 22 - DNA, 174 bp, Artificial Sequence
  Fusion sequence
gcagtctctggcagaaaaggaaactcacttgactaatcttcgggcagagagaaggaaacacttagaggaagttctggagatgaaccttgccctttaaggtggtggtgatctcagccatcctggccctggtggtgctcaccatcatctcccttatcatcctcatcatgctttggc

SEQ ID NO: 23 - DNA, 159 bp, Artificial Sequence
  Fusion sequence
aaagaagagtgcacaaatgttagaggaggcgcgacgacgggaggacaatctcaacgacagctctcagcagctacagaaagccttacagaaatctccaggacctcagcgagaaaggaagtcatcttcatcctcagaagacaggaatcgaatgaaaacact

SEQ ID NO: 24 - DNA, 140 bp, Artificial Sequence
  Fusion sequence
ccagcttcctataacttggacgatgaccaggcggcttgggagaatgagctgcagaagatgacccgggggcagccagcagatgaagatcatcgaaatcaatttgggcaacgagaccgatcctcatcagctcccaatgtgca

SEQ ID NO: 25 - DNA, 147 bp, Artificial Sequence
  Fusion sequence
aaagaagagtgcacaaatgttagaggaggcgcgacgacgggaggacaatctcaacgacagctctcagcagctacaggaggatccaaagtgggaattccctcggaagaacttggttcttggaaaaactctaggagaaggcgaatttgg

SEQ ID NO: 26 - DNA, 143 bp, Artificial Sequence
  Fusion sequence
gctggagagatacatgacctcaaggacatgttggatgtgaaggagcggaaggttaatgttcttcagaagaaggaggatccaaagtgggaattccctcggaagaacttggttcttggaaaaactctaggagaaggcgaatttgg

SEQ ID NO: 27 - DNA, 150 bp, Artificial Sequence
  Fusion sequence
gggtcaaagatccttgcatctaatagtatcatctgccctaatcactttacccctaggcggggctgccgaaatcatgagcatgttaatgttagctggtgctttgtgtgctcagaaggcatagacgtctcttccctttgccacaatggaggc

SEQ ID NO: 28 - DNA, 141 bp, Artificial Sequence
  Fusion sequence
gcttactgaccaacctggcagacagggagctggttcacatgatcaactgggcgaagagggtgccagaaaaatggctactggacaggatcgagtggttgctctcgtggacatggactgtttttttgttcaagtggagcagcg

SEQ ID NO: 29 - DNA, 101 bp, Artificial Sequence
  Fusion sequence
gcttctccgccatgcaaggctggcgcgtctccatggagtgatggaaggccacggggaagtgaatattaagcattatctaaactgcagtcactgtgaggtag

SEQ ID NO: 30 - DNA, 123 bp, Artificial Sequence
  Fusion sequence
ctgcagaaagacttgaaggcgtatacaggaacaatattgatgatgtagtaaggctagatcgcctttttattttactggatactggcactactcctgttacaagaaaagctgctgcacagcaac

SEQ ID NO: 31 - DNA, 150 bp, Artificial Sequence
  Fusion sequence
ctgcagaaagacttgaaggcgtatacaggaacaatattgatgatgtagtaaggaggaacgcccctgtcagtaggccgcactcatggcatgtggccaagctgctggagggatgccctgaagcagccaccaccatgcatttcccttctgaag

SEQ ID NO: 32 - DNA, 165 bp, Artificial Sequence
  Fusion sequence
ctgcagaaagacttgaaggcgtatacaggaacaatattgatgatgtagtaaggtttttggattcaaagcataaaaaccattacaagatatacaatcttgacgtgtgtgtgcagtggtgtccactctcccggcattgcagcaccgagaaaagcagctccattggca

SEQ ID NO: 33 - DNA, 138 bp, Artificial Sequence
  Fusion sequence
caacagctcattaaaaggcaccaagaatttgtacacatgtacaatgcccaatgcgatgctttgcatcctaaatcaggatcaaccacaggtttgtctgctaccccccctgcctcattacctggctcactaactaacgtg

SEQ ID NO: 34 - DNA, 151 bp, Artificial Sequence
  Fusion sequence
gctcggagttggcattcatcattttctaatcatcagcatttacatgacagaaatcacctatgtttacagcgacaggttatcttgacgaatcagattacaacccatctgagtggagccctggcttctcaggcagacctggtgtctccagctg

SEQ ID NO: 35 - DNA, 150 bp, Artificial Sequence
  Fusion sequence
ctcgcatcgcgcactttttggatcggcatctagtctttccgcttcttgaatttctctctgtaaaggagattacaggtccaccaggttgtggaaaaactcagttttgtataatgatgagcattttggctacattacccaccaacatgggag

SEQ ID NO: 36 - DNA, 168 bp, Artificial Sequence
  Fusion sequence
ctaaagcagctcaaaagaaagcagaagccactggagaaaaacggccaagaggcagacctaggaaatggagacaacattttgctctgtcacccaagctgaactgaactgggctccagaaatcctcccacctcagcctcctgagcagctaggactacagatgtgccacca

SEQ ID NO: 37 - DNA, 150 bp, Artificial Sequence
  Fusion sequence
gttatccgcgatgcgtttcctggcagctacattcctgctcctggcgctcagcaccgctgcccaggccgaaccggtgcagttcaaggactgcggcacttctggatccagctgtgtgatagccgcactaggaaatacctggagtcacagtgt

SEQ ID NO: 38 - DNA, 151 bp, Artificial Sequence
  Fusion sequence
caggccaccttcgtgaacgcgctgcacctctacctgtggctctttctgctgggcctgcccttcaccctctacatggttatcttgacgaatcagattacaacccatctgagtggagccctggcttctcaggcagacctggtgtctccagctg

SEQ ID NO: 39 - DNA, 199 bp, Artificial Sequence
  Fusion sequence
ctgagcacccagaattagaacatatcatctggacccttttccagcacaccctgcagaatgagtatgaactcatgagagacaggcatttggaccaaaatcttagtatcaattggtgaatcattcgggacttctgagaagttccagaaaataaatcagatggtatgtaacagcgaccgtgtgctcaaaagaagtgctgaag

SEQ ID NO: 40 - DNA, 148 bp, Artificial Sequence
  Fusion sequence
tgttccatgtatggcatatgcaaagtgaagaatatagaccttaaattcaaaatcattgtaacagcatacaaggatcttcctcatgctgttcaggagccccctaccttgtcaccaatacctcacattcctcgaagcccttacaagtttc

SEQ ID NO: 41 - DNA, 169 bp, Artificial Sequence
  Fusion sequence
tgttccatgtatggcatatgcaaagtgaagaatatagaccttaaattcaaaatcattgtaacagcatacaaggatcttcctcatgctgttcaggagacttctgagaagttccagaaaataaatcagatggtatgtaacagcgaccgtgtgctcaaaagaagtgctgaag

SEQ ID NO: 42 - DNA, 149 bp, Artificial Sequence
  Fusion sequence
aatgctatgtcaagactgttgaagaagtatgatgtattgtttgcactcttcagcaaattggaaaggacatgtgaacttatatatttgacacaacccagcagttcgatatctactgaaataaattctgcattggtgctaaaagtttcttg

SEQ ID NO: 43 - DNA, 148 bp, Artificial Sequence
  Fusion sequence
aggatcagatgaagcagatggaagtaaacatctcccaggagagtccaaatttcagcagaaactggcagaaatgacttctactcgaacacgaatgcaaaagcagaaaatgaatgatagcatggatacctcaaacaaggaagagaaatga

SEQ ID NO: 44 - DNA, 262 bp, Artificial Sequence
  Fusion sequence
tgttccatgtatggcatatgcaaagtgaagaatatagaccttaaattcaaaatcattgtaacagcatacaaggatcttcctcatgctgttcaggagacattcaaacgtgttttgatcaaagaagaggagtatgattctattatagtattctataactcggtcttcatgcagagactgaaaacaaatattttgcagtatgcttccaccaggccccctaccttgtcaccaatacctcacattcctcgaagcccttacaagtttc

SEQ ID NO: 45 - DNA, 148 bp, Artificial Sequence
  Fusion sequence
attccagggcggatggtggtgatggaagcaggtggaattatgccctgttggttccaatgctgggattggctgcttttcgggttggctgtgttccggccgcagagcaccgtctgcgtgaggagatcctggccaagttcctgcactggct

SEQ ID NO: 46 - DNA, 128 bp, Artificial Sequence
  Fusion sequence
ctataaactgctgatccacatgagagtccactctggggagaagcccaacaagtgtacggggttggctgtgttccggccgcagagcaccgtctgcgtgaggagatcctggccaagttcctgcactggct

SEQ ID NO: 47 - DNA, 130 bp, Artificial Sequence
  Fusion sequence
caaaggcaacatgaagtacaaagcagtgagtgtcaacgaaggctataaagtctgtgagaggggttggctgtgttccggccgcagagcaccgtctgcgtgaggagatcctggccaagttcctgcactggct

SEQ ID NO: 48 - DNA, 146 bp, Artificial Sequence
  Fusion sequence
gcagcagccagcctgatacgatttccatcgcctcacggacgtctcagaacacgctggacagcgataaggtgtcctgcctgaaggagctggtggcccgagtgctgcagaggctgtgcgagcgcggcgcgaagaacgtgctggccttc

SEQ ID NO: 49 - DNA, 195 bp, Artificial Sequence
  Fusion sequence
catgcccaccaacttcaccgtggtgcccgtggaggctcacgccgacggcggcggggacgagactgccgagcggacggaggctccgggcacccccgagggccccgagcccgagcgccccagcccgggggttggctgtgttccggccgcagagcaccgtctgcgtgaggagatcctggccaagttcctgcactggct

SEQ ID NO: 50 - DNA, 93 bp, Artificial Sequence
  Fusion sequence
cgggctgggctttcctcacccgggggttggctgtgttccggccgcagagcaccgtctgcgtgaggagatcctggccaagttcctgcactggct

SEQ ID NO: 51 - DNA, 130 bp, Artificial Sequence
  Fusion sequence
acggcctattcccctggtgcggcctgctgctggatacccggaccctggaggtgcagagcgactactccagttggacagtgctccagggaagaatcgggcgtccagacaacccatttcgagtggccctgga

SEQ ID NO: 52 - DNA, 150 bp, Artificial Sequence
  Fusion sequence
agtaaatatcgcagctgcacgattaatgaagatacaggtcttggactggccttcaccattgcccatgagtctggacacaagggttggctgtgttccggccgcagagcaccgtctgcgtgaggagatcctggccaagttcctgcactggct

SEQ ID NO: 53 - DNA, 123 bp, Artificial Sequence
  Fusion sequence
gagctgaatgaggccttggaactcaaggatgcccaggctgggaaggagccaggggggagcagggctcactccagtcccggccagtgtgcagctgcacacggcggtggagatgcaccactggtg

SEQ ID NO: 54 - DNA, 118 bp, Artificial Sequence
  Fusion sequence
cagaggaagagaatctccgcaagaaaggggagcctcaccacgagctgcccccagggagcactaagcgagtcccggccagtgtgcagctgcacacggcggtggagatgcaccactggtg

SEQ ID NO: 55 - DNA, 104 bp, Artificial Sequence
  Fusion sequence
ctcctctccccagccaaagaagaaaccactggatggagaatatttcacccttcagtcccggccagtgtgcagctgcacacggcggtggagatgcaccactggtg

SEQ ID NO: 56 - DNA, 139 bp, Artificial Sequence
  Fusion sequence
tcccctccttctccctttttatatcccatttttatatcgatctcttattttacaataaaactttgctgccacctgtgtgtctgaggggtgtcccggccagtgtgcagctgcacacggcggtggagatgcaccactggtg

SEQ ID NO: 57 - DNA, 125 bp, Artificial Sequence
  Fusion sequence
caagcgaggtcgcaagtctgccacagtaaaacctgccttgccctttaaggtggtggtgatctcagccatcctggccctggtggtgctcaccatcatctcccttatcatcctcatcatgctttggc

SEQ ID NO: 58 - DNA, 161 bp, Artificial Sequence
  Fusion Sequence
gtgctgtctcaattgcaggaaaagaaactctttcatctgctgctaaaagtgcttcaagggccaggctgccaggccatgttgcagctgaccacccacctgcagtgtaccgccggaagcaccaggagctgcaagccatgcagatggagctgcagagccctgag

SEQ ID NO: 59 - DNA, 149 bp, Artificial Sequence
  Fusion protein
ccaggaggtggtggagacaatgtgcccagtgcccgctgcagctaccagcaacgtccatatggtgaagaagattagcatcacagagcgaagctgcgatggagcagcaggtggtggaggtggctggaatgataacacttccttgctctggg

SEQ ID NO: 60 - DNA, 147 bp, Artificial Sequence
  Fusion sequence
atcgcaaacgctatcagcaagaagtagatcgcataaaggaagcagtcaggtcaaagaatatggccagaagagggcattctgcacagattgtgtcacccaccccggagccacacctgccactctcgctgatcctctctgtggtgacct

SEQ ID NO: 61 - DNA, 129 bp, Artificial Sequence
  Fusion sequence
ggaccagctccggcatgcggtcccagtggccctcggcgcggcagcgctccagctcgctctccaccttcagtgtaccgccggaagcaccaggagctgcaagccatgcagatggagctgcagagccctgag

SEQ ID NO: 62 - DNA, 131 bp, Artificial Sequence
  Fusion sequence
cccgctgaatggacacctgcagcactgtttggagactgtccgggaggaattcatcatattcaacagagagaatgtaccgccggaagcaccaggagctgcaagccatgcagatggagctgcagagccctgag

SEQ ID NO: 63 - DNA, 124 bp, Artificial Sequence
  Fusion sequence
gcttcctgatggccgcttccatctgccgccccgaatccatcgagcctccgacccaggtttgccagtgtaccgccggaagcaccaggagctgcaagccatgcagatggagctgcagagccctgag

SEQ ID NO: 64 - DNA, 175 bp, Artificial Sequence
  Fusion sequence
ctcagcgtgatccctttacagttgctggctttccaccttgctgtgctgagaggctatgatgacctcctccatcagtgacctgaaggaggtgccgcggaaaaacatcaccctcattcggggtctgggccatggcgcctttggggaggtgtatgaaggccaggtgtccggaatgccc

SEQ ID NO: 65 - DNA, 136 bp, Artificial Sequence
  Fusion sequence
ggaagaattagaagctgagaaaagagacttaattagaaccaatgagagactatctcaagaacttgaatacttaacagtgtaccgccggaagcaccaggagctgcaagccatgcagatggagctgcagagccctgag

SEQ ID NO: 66 - DNA, 137 bp, Artificial Sequence
  Fusion sequence
ccttcctggctgtaggatctcatgacaactttatttacctctatgtagtctctgaaaatggaagaaaatatagcagatatggaaggtgcactcaccaggagctgcaagccatgcagatggagctgcagagccctgag

SEQ ID NO: 67 - DNA, 149 bp, Artificial Sequence
  Fusion sequence
tggaaatggcacagaaacaaagtatggatgaatcattacatcttggctgggaactggaacagatatccagaactagtgaactttccgaagtgtaccgccggaagcaccaggagctgcaagccatgcagatggagctgcagagccctgag

SEQ ID NO: 68 - DNA, 130 bp, Artificial Sequence
  Fusion sequence
acacagttacagcagttgcttcaggcggtaaaccaacagctcacaaaggagaaagagcactaccaggtgttagtgtcacccaccccggagccacacctgccactctcgctgatcctctctgtggtgacct

SEQ ID NO: 69 - DNA, 166 bp, Artificial Sequence
  Fusion sequence
ctcgaatctcccagctggagatggcccgacagaagaaggagagtgaggctgtggagtggcagcagaagcaggcagcatgggagaaggcactcatggtttcgtcaaatacttactggagttctctcaggagctgcaagccatgcagatggagctgcagagccctgag

SEQ ID NO: 70 - DNA, 129 bp, Artificial Sequence
  Fusion sequence
ctggagatggacctgaaggacctggaggcgcacatcgactcggccaacaagaaccgggacgaagccatcaaacagctgcggaagctgcaggtcctgcaagccatgcagatggagctgcagagccctgag

SEQ ID NO: 71 - DNA, 125 bp, Artificial Sequence
  Fusion sequence
ggatgttattaactggaggagggaaagacagaaaaataattctgtgggatcatgatctgaatcctgaaagagaaatagagcaccaggagctgcaagccatgcagatggagctgcagagccctgag

SEQ ID NO: 72 - DNA, 101 bp, Artificial Sequence
  Fusion sequence
ggatgttattaactggaggagggaaagacagaaaaataattctgtgggatcatgatctgaatcctgaaagagaaatagagatggagctgcagagccctgag

SEQ ID NO: 73 - DNA, 215 bp, Artificial Sequence
  Fusion sequence
gacactgtgcagattttcatccaagtggcacagtggtggccataggaacgcactcaggcaggagacaaaaacatgaagtcaattttcccaaaattaaactcattaaaaaatgtggaatgctgccaggccatgttgcagctgaccacccacctgcagtgtaccgccggaagcaccaggagctgcaagccatgcagatggagctgcagagccctgag

SEQ ID NO: 74 - DNA, 130 bp, Artificial Sequence
  Fusion sequence
gtctgaaatgaaggagctccgtgaccttaaccggaggctccaggacgtgctgctcctgcggcttggcagcgtgtaccgccggaagcaccaggagctgcaagccatgcagatggagctgcagagccctgag

SEQ ID NO: 75 - DNA, 184 bp, Artificial Sequence
  Fusion sequence
tgcttcagaatcactgagaaaagaagaagaacaagctacagagacacaacccattgtttatgatggggtctcgtctgtcacccaggctggagtgcagtggcgtgatctcggctcactgcaacctttgtaccgccggaagcaccaggagctgcaagccatgcagatggagctgcagagccctgag

SEQ ID NO: 76 - DNA, 145 bp, Artificial Sequence
  Fusion sequence
ctgcagcctgcatctgagtacaccgtatccctcgtggccataaagggcaaccaagagagccccaaagccactggagtctttaccacactgtcacccaccccggagccacacctgccactctcgctgatcctctctgtggtgacct

SEQ ID NO: 77 - DNA, 144 bp, Artificial Sequence
  Fusion sequence
gcgggtgcagaactacctgtacaacgtgctggagagaccccgcggctgggcgttcatctaccacgctttcgtgttctggctgcagatggtcgcatggtggggacaaggatccagagccatcgtggcttttgacaatatctccat

SEQ ID NO: 78 - DNA, 132 bp, Artificial Sequence
  Fusion sequence
accacaggaaagcagtgtcaagattgtatgccaggttattatggagatccaaccaatggtggacagtgcacagtgtaccgccggaagcaccaggagctgcaagccatgcagatggagctgcagagccctgag

SEQ ID NO: 79 - DNA, 150 bp, Artificial Sequence
  Fusion sequence
ccagccttggaagactatgtagtattgatgaaaagagggactgccataacattcccaaagctccgaatgtcctggctcattcgtggagtcttgaggggaaacgtgtccttggtgctagtggagaacaaaaccgggaaggagcaaggcagg

SEQ ID NO: 80 - DNA, 125 bp, Artificial Sequence
  Fusion sequence
cggctatggtgcacagcagccgcaggctccacctcagcagcctcaacagtatggtattcagtattcagtgtcacccaccccggagccacacctgccactctcgctgatcctctctgtggtgacct

SEQ ID NO: 81 - DNA, 100 bp, Artificial Sequence
  Fusion sequence
cgtcataagaccgcgaccagacaggcggcgccatcttcgaacttagacttccggaaggactttggcgaggattatctaaactgcagtcactgtgaggtag

SEQ ID NO: 82 - DNA, 165 bp, Artificial Sequence
  Fusion sequence
gttagtgaaatggacagtgagagacttcagtatgaaaaaaagcttaaatcaaccaaagttactacgtgctcggcaatttacacatttcaattcattcgatcctcagtgtaccgccggaagcaccaggagctgcaagccatgcagatggagctgcagagccctgag

SEQ ID NO: 83 - DNA, 155 bp, Artificial Sequence
  Fusion sequence
ggacaaggacatgttccaggagaccatggaggccatgaggattatgggcatcccagaagaggagcaaatggtgctctccaggaacatccccaggctccaagatggccctgcagagctccttcacttgttggaatgggacagtcctccagcttggg

SEQ ID NO: 84 - DNA, 131 bp, Artificial Sequence
  Fusion sequence
gccagctgctggagacattgaatcaattgagcacacacacgcacgtagtagacatcactcgcaccagccctgtgtaccgccggaagcaccaggagctgcaagccatgcagatggagctgcagagccctgag

SEQ ID NO: 85 - DNA, 124 bp, Artificial Sequence
  Fusion sequence
aaaactgggagagcttcggaaaaagcactacgagcttgctggtgttgctgagggctgggaagaagtgtaccgccggaagcaccaggagctgcaagccatgcagatggagctgcagagccctgag

SEQ ID NO: 86 - DNA, 123 bp, Artificial Sequence
  Fusion sequence
caaaaggaggaacagtttaacatgctgtcttctgacttggagaagctgagagaaaacttagcagtgtaccgccggaagcaccaggagctgcaagccatgcagatggagctgcagagccctgag

SEQ ID NO: 87 - DNA, 130 bp, Artificial Sequence
  Fusion sequence
tgaggaacaggacacatttgtttctgtgatttactgttctggcacagacaggctgtgtgcatgcaccaaagtgtaccgccggaagcaccaggagctgcaagccatgcagatggagctgcagagccctgag

SEQ ID NO: 88 - DNA, 137 bp, Artificial Sequence
  Fusion sequence
ggagagagagtgttagtggtaggacagagactgggcaccattaggttctttgggacaacaaacttcgctccaggccccggttcatcctgctggagctcatggcggggggagacctcaagtccttcctccgagagacc

SEQ ID NO: 89 - DNA, 160 bp, Artificial Sequence
  Fusion sequence
gtcgaaaataccttcaacacccaaattaataccaaaagttaccaaaactgcagacaagcataaagatgtcatcatcaaccaagctgaccacccacctgcagtgtaccgccggaagcaccaggagctgcaagccatgcagatggagctgcagagccctgag

SEQ ID NO: 90 - DNA, 146 bp, Artificial Sequence
  Fusion sequence
ggaatggagatgttcttactggagactcaggtggagtcatgcttatatggagcaaaactactgtagagcccacacctgggaaaggacctaaagatccagggaggcttcctgtaggaagtggcctgtgtagtgcttcaagggccagg

SEQ ID NO: 91 - DNA, 168 bp, Artificial Sequence
  Fusion sequence
ggatgttattaactggaggagggaaagacagaaaaataattctgtgggatcatgatctgaatcctgaaagagaaatagaggtccctgagtacaagctgagcaagctccgcacctcgaccatcatgaccgactacaaccccaactactgctttgctggcaagacctcct

SEQ ID NO: 92 - DNA, 166 bp, Artificial Sequence
  Fusion sequence
ggatgttattaactggaggagggaaagacagaaaaataattctgtgggatcatgatctgaatcctgaaagagaaatagagggaaaggttcagagctcaggggaggatatggagatccagggaggcttcctgtaggaagtggcctgtgtagtgcttcaagggccagg

SEQ ID NO: 93 - DNA, 185 bp, Artificial Sequence
  Fusion sequence
gacactgtgcagattttcatccaagtggcacagtggtggccataggaacgcactcaggcagagtaacagattccctggataccctttcagaaatttcttcaaataaacagaaccattcttatcctgtgtaccgccggaagcaccaggagctgcaagccatgcagatggagctgcagagccctgag

SEQ ID NO: 94 - DNA, 188 bp, Artificial Sequence
  Fusion sequence
gacactgtgcagattttcatccaagtggcacagtggtggccataggaacgcactcaggcagagtcttgctctgtctcccaggctggagtgcagtggcaatttacacatttcaattcattcgatcctcagtgtaccgccggaagcaccaggagctgcaagccatgcagatggagctgcagagccctgag

SEQ ID NO: 95 - DNA, 150 bp, Artificial Sequence
  Fusion sequence
gacactgtgcagattttcatccaagtggcacagtggtggccataggaacgcactcaggcaggccatgttgcagctgaccacccacctgcagtgtaccgccggaagcaccaggagctgcaagccatgcagatggagctgcagagccctgag

SEQ ID NO: 96 - DNA, 176 bp, Artificial Sequence
  Fusion sequence
gcactgctcgacctgagagacccatggcattcctcagggaatactttgagaggttggagaagacctcctccatcagtgacctgaaggaggtgccgcggaaaaacatcaccctcattcggggtctgggccatggcgcctttggggaggtgtatgaaggccaggtgtccggaatgccc

SEQ ID NO: 97 - DNA, 148 bp, Artificial Sequence
  Fusion sequence
cgatggcactttcctgtggaagatcaccaatgtcaccaggcggtgccatgagtcggcctgtggcaggaccgtcagcctcttctccccagtgtaccgccggaagcaccaggagctgcaagccatgcagatggagctgcagagccctgag

SEQ ID NO: 98 - DNA, 169 bp, Artificial Sequence
  Fusion sequence
gcaccacttttgaccaatacttcagaagacaaatgtgaaaaggataatatagttggatcaaacaaaaacaacacaatttgtcccggtcatagctccttggaatcaccaacaaacatgccttctccttctcctgattattttacatggaatctcacctggataatgaaag

SEQ ID NO: 99 - DNA, 163 bp, Artificial Sequence
  Fusion sequence
agaactaaagcagcgtctgaacagccagtccaaacgcacgattgagggactccggggccctcctccttcaggcattgctactctggtctctggcattgctggtgtgtaccgccggaagcaccaggagctgcaagccatgcagatggagctgcagagccctgag

SEQ ID NO: 100 - DNA, 149 bp, Artificial Sequence
  Fusion sequence
caaatggctgcaaactaatcaggaatcgatcggattgtaaggacattgattggacgacatatacctgtgtgctaggatttcaagtatttgtgtaccgccggaagcaccaggagctgcaagccatgcagatggagctgcagagccctgag

SEQ ID NO: 101 - DNA, 135 bp, Artificial Sequence
  Fusion sequence
cagtcatcaagcctctgttggttgaacctgaaggactagagaaggaaacaacattcaactccctactttgtccatcagtgtcacccaccccggagccacacctgccactctcgctgatcctctctgtggtgacct

SEQ ID NO: 102 - DNA, 128 bp, Artificial Sequence
  Fusion sequence
ctgagactcgggctgagtttgcggagaggtcagtaactaaattggagaaaagcattgatgacttagaagtgtaccgccggaagcaccaggagctgcaagccatgcagatggagctgcagagccctgag

SEQ ID NO: 103 - DNA, 149 bp, Artificial Sequence
  Fusion sequence
aaatgcagcttgttgattccatagttcgtcagcgtgatatgtaccgtattttattgtcacaaacaacaggagttgccattccattacatgtgtaccgccggaagcaccaggagctgcaagccatgcagatggagctgcagagccctgag

SEQ ID NO: 104 - DNA, 125 bp, Artificial Sequence
  Fusion sequence
ctcaaaacagaagcagatggaacccagcaggtgcaacaggttcaggtgtttgctgacgtccagtgtacagtgaatctggtaggcggctgtggggctgctccagttcaatctcagcgagctgttca

SEQ ID NO: 105 - DNA, 117 bp, Artificial Sequence
  Fusion sequence
acagctagaaggttggctttcacaagtacagtctacaaaaagacctgctagagccattattgccccggaaactgcctgtgggtttttactgcaactttgaagatggcttctgtggct

SEQ ID NO: 106 - DNA, 123 bp, Artificial Sequence
  Fusion sequence
cctctcatccagaacgtccatgcctccaagcgcattctcttctccatcgtccatgacaagtcagtgtaccgccggaagcaccaggagctgcaagccatgcagatggagctgcagagccctgag

SEQ ID NO: 107 - DNA, 120 bp, Artificial Sequence
  Fusion sequence
agcgacgccattgctcatggtgccaccacctgcctcagagccccacctgagcctgccgacttgtaccgccggaagcaccaggagctgcaagccatgcagatggagctgcagagccctgag

SEQ ID NO: 108 - DNA, 107 bp, Artificial Sequence
  Fusion sequence
gcgttgtggcctcaaccatttccggcaaatcacagatcgaagagacagtgtaccgccggaagcaccaggagctgcaagccatgcagatggagctgcagagccctgag

SEQ ID NO: 109 - DNA, 125 bp, Artificial Sequence
  Fusion sequence
ggaatggagatgttcttactggagactcaggtggagtcatgcttatatggagcaaaactactgtagagcccacacctgggaaaggacctaaaggaagtggcctgtgtagtgcttcaagggccagg

SEQ ID NO: 110 - DNA, 180 bp, Artificial Sequence
  Fusion sequence
ggatgttattaactggaggagggaaagacagaaaaataattctgtgggatcatgatctgaatcctgaaagagaaatagagatatgctggatgagccctgagtacaagctgagcaagctccgcacctcgaccatcatgaccgactacaaccccaactactgctttgctggcaagacctcct

SEQ ID NO: 111 - DNA, 176 bp, Artificial Sequence
  Fusion sequence
gacactgtgcagattttcatccaagtggcacagtggtggccataggaacgcactcaggcagcatactatgtatacaagggagttgcagagccctgagtacaagctgagcaagctccgcacctcgaccatcatgaccgactacaaccccaactactgctttgctggcaagacctcct

SEQ ID NO: 112 - DNA, 159 bp, Artificial Sequence
  Fusion sequence
gacactgtgcagattttcatccaagtggcacagtggtggccataggaacgcactcaggcagggagttgcagagccctgagtacaagctgagcaagctccgcacctcgaccatcatgaccgactacaaccccaactactgctttgctggcaagacctcct

SEQ ID NO: 113 - DNA, 162 bp, Artificial Sequence
  Fusion sequence
cattccagctacatcacacaccttgactggtccccagacaacaagtatataatgtctaactcgggagactatgaaatattgtactctgaccacccacctgcagtgtaccgccggaagcaccaggagctgcaagccatgcagatggagctgcagagccctgag

SEQ ID NO: 114 - DNA, 162 bp, Artificial Sequence
  Fusion sequence
gcggctttggctgatgttttgaggcgtcttgcaatctctgaagatcatgtggcctcagtgaaaaaatcagtctcaagtaaaggttcagagctcaggggaggatatggagatccagggaggcttcctgtaggaagtggcctgtgtagtgcttcaagggccagg

SEQ ID NO: 115 - DNA, 135 bp, Artificial Sequence
  Fusion sequence
gtcgaaaataccttcaacacccaaattaataccaaaagttaccaaaactgcagacaagcataaagatgtcatcatcaaccaaggcggcaatgcagcctcaaacaatgaccccgaaatggatggggaagatggggt

SEQ ID NO: 116 - DNA, 146 bp, Artificial Sequence
  Fusion sequence
gtcgaaaataccttcaacacccaaattaataccaaaagttaccaaaactgcagacaagcataaagatgtcatcatcaaccaagtgatggaaggccacggggaagtgaatattaagcattatctaaactgcagtcactgtgaggtag

SEQ ID NO: 117 - DNA, 175 bp, Artificial Sequence
  Fusion sequence
gtcgaaaataccttcaacacccaaattaataccaaaagttaccaaaactgcagacaagcataaagatgtcatcatcaaccaagcaaaaatgtcaactcgcgaaaaaaacagccaagtgtaccgccggaagcaccaggagctgcaagccatgcagatggagctgcagagccctgag

SEQ ID NO: 118 - DNA, 143 bp, Artificial Sequence
  Fusion sequence
aattaccatgttcattccttccgatgttgacaactatgatgacatcagaacggaactgcctcctgagaagctgagcaagctccgcacctcgaccatcatgaccgactacaaccccaactactgctttgctggcaagacctcct

SEQ ID NO: 119 - DNA, 131 bp, Artificial Sequence
  Fusion sequence
caggcttcgcaggagatgatgccccccgggctgtcttcccctccattgtgggccgccctcgccaccagtgatggaaggccacggggaagtgaatattaagcattatctaaactgcagtcactgtgaggtag

SEQ ID NO: 120 - DNA, 140 bp, Artificial Sequence
  Fusion sequence
tggattttgccatgccctatttcatccaggtcatgaaggagtacttgacaaagggtgaaggttcagagctcaggggaggatatggagatccagggaggcttcctgtaggaagtggcctgtgtagtgcttcaagggccagg

SEQ ID NO: 121 - DNA, 141 bp, Artificial Sequence
  Fusion sequence
tgtctccaccaacaaacttgcatctggaggcaaaccctgacactggagtgctcacagtctcctgggagaggagcaccaccccagtgtcacccaccccggagccacacctgccactctcgctgatcctctctgtggtgacct

SEQ ID NO: 122 - DNA, 149 bp, Artificial Sequence
  Fusion sequence
gaagggaggaactgttacttctaaagaaagagaaaagatgtgttgatagtctcctgaagatgtgtgggaacgtgaaacatctgatacaagtgtaccgccggaagcaccaggagctgcaagccatgcagatggagctgcagagccctgag

SEQ ID NO: 123 - DNA, 129 bp, Artificial Sequence
  Fusion sequence
gatcttcgacagtgcctgaacaggtacaagaaaatgcaagacacggtggtactggcccaaggtaaaaaagtgtaccgccggaagcaccaggagctgcaagccatgcagatggagctgcagagccctgag

SEQ ID NO: 124 - DNA, 140 bp, Artificial Sequence
  Fusion sequence
cttctggtccatcggaggatccgagtgtgaatttcctgaagaacgttggggagagtgtggcagctgcccttagccctctggtgtaccgccggaagcaccaggagctgcaagccatgcagatggagctgcagagccctgag

SEQ ID NO: 125 - DNA, 140 bp, Artificial Sequence
  Fusion sequence
aggaaagagccaaataccacaagttgaaatacgggacagaattgaatcagggagatatgaagcctccaagctatgattctgtgtaccgccggaagcaccaggagctgcaagccatgcagatggagctgcagagccctgag

SEQ ID NO: 126 - DNA, 127 bp, Artificial Sequence
  Fusion sequence
ctgacaaactgaaagaggctgagacccgtgctgaatttgcagagagaacggttgcaaaactggaaaagtgtaccgccggaagcaccaggagctgcaagccatgcagatggagctgcagagccctgag

SEQ ID NO: 127 - DNA, 149 bp, Artificial Sequence
  Fusion sequence
ctgtgaaagctgcctctgatgaattgagcaaaaccatctccccgatggtgatggatgcaaaagctgtggctggaaacatttccgaccctgtgtaccgccggaagcaccaggagctgcaagccatgcagatggagctgcagagccctgag

SEQ ID NO: 128 - DNA, 136 bp, Artificial Sequence
  Fusion sequence
gagcagctgagatgatggcatctttactaaaagaccttgcagaaataggaattgctgtgggaaataatgatgtaaagtgtaccgccggaagcaccaggagctgcaagccatgcagatggagctgcagagccctgag

SEQ ID NO: 129 - DNA, 122 bp, Artificial Sequence
  Fusion sequence
gagcagctgagatgatggcatctttactaaaagaccttgcagaaataggaattgctgtgggaaataatgatgtaaagcaccaggagctgcaagccatgcagatggagctgcagagccctgag

SEQ ID NO: 130 - DNA, 127 bp, Artificial Sequence
  Fusion sequence
aaagaaaagacagttggaggaatctgtcgatgccctcagtgaagaactagtccagcttcgagcacaagtgtaccgccggaagcaccaggagctgcaagccatgcagatggagctgcagagccctgag

SEQ ID NO: 131 - DNA, 149 bp, Artificial Sequence
  Fusion sequence
atcgcaaacgctatcagcaagaagtagatcgcataaaggaagcagtcaggtcaaagaatatggccagaagagggcattctgcacagattgtgtaccgccggaagcaccaggagctgcaagccatgcagatggagctgcagagccctgag

SEQ ID NO: 132 - DNA, 143 bp, Artificial Sequence
  Fusion sequence
cctcctcagcagctcacccaccaggcgttcagccacagcagccaccatatacaggagctcagactcaagcaggtcagattgaagtgtaccgccggaagcaccaggagctgcaagccatgcagatggagctgcagagccctgag

SEQ ID NO: 133 - DNA, 130 bp, Artificial Sequence
  Fusion sequence
agtgaatcgtttattggatagcttggaaccacctggagaaccaggaccttccaccaatattcctgaaaatgtgtaccgccggaagcaccaggagctgcaagccatgcagatggagctgcagagccctgag

SEQ ID NO: 134 - DNA, 105 bp, Artificial Sequence
  Fusion sequence
aaaaatgttatgtcagcgtttggcttaacagatgatcaggtttcagtgtaccgccggaagcaccaggagctgcaagccatgcagatggagctgcagagccctgag

SEQ ID NO: 135 - DNA, 128 bp, Artificial Sequence
  Fusion sequence
cagagacccgtgctgagtttgctgagagatcggtagccaagctggaaaagacaattgatgacctggaagtgtaccgccggaagcaccaggagctgcaagccatgcagatggagctgcagagccctgag

SEQ ID NO: 136 - DNA, 134 bp, Artificial Sequence
  Fusion sequence
caaatgctgctggtcagcttcccacatctccaggtcatatgcacacccaggtaccaccttatccacagccacagctgtaccgccggaagcaccaggagctgcaagccatgcagatggagctgcagagccctgag

SEQ ID NO: 137 - DNA, 150 bp, Artificial Sequence
  Fusion sequence
gctccaccatcatcttcagcttatgcactgcctcctggaacaacaggtacactgcctgctgccagtgagctgcctgcgtcccaaagaacagtgtaccgccggaagcaccaggagctgcaagccatgcagatggagctgcagagccctgag

SEQ ID NO: 138 - DNA, 147 bp, Artificial Sequence
  Fusion sequence
catcgttggcccacagagaattatggaccagactcagtgcctgatggatatcaggggtcacagacatttcatggggctccactaacagtgtaccgccggaagcaccaggagctgcaagccatgcagatggagctgcagagccctgag

SEQ ID NO: 139 - DNA, 142 bp, Artificial Sequence
  Fusion sequence
gggctttgaaataacaccaccagtggtcttaaggttgaagtgtggttcagggccagtgcatattagtggacagcacttagtagtgtaccgccggaagcaccaggagctgcaagccatgcagatggagctgcagagccctgag

SEQ ID NO: 140 - DNA, 110 bp, Artificial Sequence
  Fusion sequence
ctcgaatctcccagctggagatggcccgacagaagaaggagagtgaggctgtggagtggcagcagaagcaggagctgcaagccatgcagatggagctgcagagccctgag

SEQ ID NO: 141 - DNA, 126 bp, Artificial Sequence
  Fusion sequence
caagcagaaacactgtacaaagagattctcactcgtgcacatgaaagggagtttggttctgtagatgtgtaccgccggaagcaccaggagctgcaagccatgcagatggagctgcagagccctgag

SEQ ID NO: 142 - DNA, 121 bp, Artificial Sequence
  Fusion sequence
tgcttcagaatcactgagaaaagaagaagaacaagctacagagacacaacccattgtttatgtgtaccgccggaagcaccaggagctgcaagccatgcagatggagctgcagagccctgag

SEQ ID NO: 143 - DNA, 152 bp, Artificial Sequence
  Fusion sequence
ggaatggagatgttcttactggagactcaggtggagtcatgcttatatggagcaaaactactgtagagcccacacctgggaaaggacctaaagtgtaccgccggaagcaccaggagctgcaagccatgcagatggagctgcagagccctgag

SEQ ID NO: 144 - DNA, 152 bp, Artificial Sequence
  Fusion sequence
tcctgaaagagaaatagaggttcctgatcagtatggcacaatcagagctgtagcagaaggaaaggcagatcaatttttagtaggcaagctccgcacctcgaccatcatgaccgactacaaccccaactactgctttgctggcaagacctcct

SEQ ID NO: 145 - DNA, 137 bp, Artificial Sequence
  Fusion sequence
tggatgcagaaaccagagatctagtttctatccacacagacgggaatgaacagctctctgtgatgcgctactcaatagtgtaccgccggaagcaccaggagctgcaagccatgcagatggagctgcagagccctgag

SEQ ID NO: 146 - DNA, 144 bp, Artificial Sequence
  Fusion sequence
cattccagctacatcacacaccttgactggtccccagacaacaagtatataatgtctaactcgggagactatgaaatattgtacttgtaccgccggaagcaccaggagctgcaagccatgcagatggagctgcagagccctgag

SEQ ID NO: 147 - DNA, 141 bp, Artificial Sequence
  Fusion sequence
gcggctttggctgatgttttgaggcgtcttgcaatctctgaagatcatgtggcctcagtgaaaaaatcagtctcaagtaaagtgtaccgccggaagcaccaggagctgcaagccatgcagatggagctgcagagccctgag

SEQ ID NO: 148 - DNA, 140 bp, Artificial Sequence
  Fusion sequence
gtcgaaaataccttcaacacccaaattaataccaaaagttaccaaaactgcagacaagcataaagatgtcatcatcaaccaagtgtcacccaccccggagccacacctgccactctcgctgatcctctctgtggtgacct

SEQ ID NO: 149 - DNA, 142 bp, Artificial Sequence
  Fusion sequence
gtcgaaaataccttcaacacccaaattaataccaaaagttaccaaaactgcagacaagcataaagatgtcatcatcaaccaagtgtaccgccggaagcaccaggagctgcaagccatgcagatggagctgcagagccctgag

SEQ ID NO: 150 - DNA, 167 bp, Artificial Sequence
  Fusion sequence
ggaaacagtacagcaaaggcgtatctcagatgcccttgagatatggaatgaacccacatcagacccctgcccagctgtacacactgcagcccaagcttcccatcacagtgtaccgccggaagcaccaggagctgcaagccatgcagatggagctgcagagccctgag

SEQ ID NO: 151 - DNA, 133 bp, Artificial Sequence
  Fusion sequence
cacagtcatgccctaccttcaggtgttatcagaattccgagaaggagtgcggaagattgcccgagagcaaaaagtgtaccgccggaagcaccaggagctgcaagccatgcagatggagctgcagagccctgag

SEQ ID NO: 152 - DNA, 135 bp, Artificial Sequence
  Fusion sequence
gccctactacctggagaacgagcccagcggctacacggtgcgcgaggccggcccgccggcattctacagtccaccaatcgattactttgatgtatttaaagaatcaaaagagcaaaatttctatgggtcacagga

SEQ ID NO: 153 - DNA, 150 bp, Artificial Sequence
  Fusion sequence
ctgtttgttcagaagacaatgttgatgttcatgatatagaattgttacagtatatcaatgtggattgtgcaaaattaaaacgactcctgaaggattctggaactgaagacatgctttggacagaaaagtatcaacctcagactgccagtg

SEQ ID NO: 154 - DNA, 140 bp, Artificial Sequence
  Fusion sequence
ctagccaaggtgtttaattcttcacattccttcttagaagacttgactggtcttacattgctgcatcaagaaacccaagctgctgaggacttgattagagaccaaggatttcgtggtgatggaggatcaaccacaggttt

SEQ ID NO: 155 - DNA, 160 bp, Artificial Sequence
  Fusion sequence
acatggatgagggtggaggttatcctgaaccccctggagctgcaggaggagctgaccccccaacccagccagaccctaaggattcctgtagctgcctcactgcggctgagattgacagacgaatatctgcatttattgaaagaaagcaagctgaaatcaa

SEQ ID NO: 156 - DNA, 150 bp, Artificial Sequence
  Fusion sequence
cactttgcgggaactggagagatatgtcaagtcttgtttacagaaaaagcaaaggaaaccgttctcatctgcattgccgggaccggatatgagcatgaaacctagtgccgccccgtctccatcccctgcacttccctttctcccaccaac

SEQ ID NO: 157 - DNA, 149 bp, Artificial Sequence
  Fusion sequence
gtcacagttccagagcctgacccaccagtctccaccccagcaaaacgtccagcctaagaaacagcatctgcattgccgggaccggatatgagcatgaaacctagtgccgccccgtctccatcccctgcacttccctttctcccaccaac

SEQ ID NO: 158 - DNA, 150 bp, Artificial Sequence
  Fusion sequence
ctcgtcctcagagtcggagagctccagtgagtccagctcctctgacagcgaagactccgaaacagcatctgcattgccgggaccggatatgagcatgaaacctagtgccgccccgtctccatcccctgcacttccctttctcccaccaac

SEQ ID NO: 159 - DNA, 133 bp, Artificial Sequence
  Fusion sequence
gccaagcctcagcaagtcatccagcaccaccattcaccccggcaccacaagtcggacccctactcaaccggtgaccgctccaaaatttccaaggacgtttatgagaacttccgtcagtggcagcgttacaaag

SEQ ID NO: 160 - DNA, 160 bp, Artificial Sequence
  Fusion sequence
cagaatgggctgtgtgaggaagaagaggcagcctcagcacctgcagttgaagaccaggtacctgaagctgccttgccctttaaggtggtggtgatctcagccatcctggccctggtggtgctcaccatcatctcccttatcatcctcatcatgctttggc

SEQ ID NO: 161 - DNA, 146 bp, Artificial Sequence
  Fusion sequence
ctcccagagaccaacgttcaagcagttggtagaagacttggatcgaattctcactctcacaaccaatgagggtggggagaaacagcgggtcttcactggtattgttaccagcttgcatgactactttggggttgtggatgaagagg

SEQ ID NO: 162 - DNA, 126 bp, Artificial Sequence
  Fusion sequence
ggagacctacaaactgaagtgcaaggcactgcaggaggagaaccgcgacctgcgcaaagccagcgtgaccatcgacttgattagagaccaaggatttcgtggtgatggaggatcaaccacaggttt

SEQ ID NO: 163 - DNA, 159 bp, Artificial Sequence
  Fusion sequence
ctcccagagaccaacgttcaagcagttggtagaagacttggatcgaattctcactctcacaaccaatgagtcgccgccgctccgagtctgcgccctggtgccaggcgctcagctcggcgctcccctgtgctcgcccggcgcccactcattcgcagcccg

SEQ ID NO: 164 - DNA, 144 bp, Artificial Sequence
  Fusion sequence
ggagacctacaaactgaagtgcaaggcactgcaggaggagaaccgcgacctgcgcaaagccagcgtgaccatcgaggatccaaagtgggaattccctcggaagaacttggttcttggaaaaactctaggagaaggcgaatttgg

SEQ ID NO: 165 - DNA, 168 bp, Artificial Sequence
  Fusion sequence
tggtttcacgccaccaacttcactgactagagctggaatgtcttattacaattccccgggtcttcacgtgcagcacatgggaacatcccatggtatcacaatctcctcagctgagatgaccttccggaggcccgcccaggccttcccggtcagctactcctcttccgg

SEQ ID NO: 166 - DNA, 116 bp, Artificial Sequence
  Fusion sequence
ggagacctacaaactgaagtgcaaggcactgcaggaggagaaccgcgacctgcgcaaagccagcgtgaccatcgagtgagctgcgagacctgctgtcagagttcaacgtcctgaag

SEQ ID NO: 167 - DNA, 97 bp, Artificial Sequence
  Fusion sequence
aggagaaagaaacccttgctgtaaattatgagaaagaagaagaattccctcggaagaacttggttcttggaaaaactctaggagaaggcgaatttgg

SEQ ID NO: 168 - DNA, 188 bp, Artificial Sequence
  Fusion sequence
tggtttcacgccaccaacttcactgactagagctggaatgtcttattacaattccccgggtcttcacgtgcagcacatgggaacatcccatggtatcacaagtttgcccacaagccacccatctcctcagctgagatgaccttccggaggcccgcccaggccttcccggtcagctactcctcttccgg

SEQ ID NO: 169 - DNA, 133 bp, Artificial Sequence
  Fusion sequence
tggtttcacgccaccaacttcactgactagagctggaatgtcttattacaccacggtggccgtgaagatgctgaaagagaacgcctccccgagtgagctgcgagacctgctgtcagagttcaacgtcctgaag

SEQ ID NO: 170 - DNA, 118 bp, Artificial Sequence
  Fusion sequence
ggagacctacaaactgaagtgcaaggcactgcaggaggagaaccgcgacctgcgcaaagtgggaattccctcggaagaacttggttcttggaaaaactctaggagaaggcgaatttgg

SEQ ID NO: 171 - DNA, 133 bp, Artificial Sequence
  Fusion sequence
ggagacctacaaactgaagtgcaaggcactgcaggaggagaaccgcgacctgcgcaaagccacccatctcctcagctgagatgaccttccggaggcccgcccaggccttcccggtcagctactcctcttccgg

SEQ ID NO: 172 - DNA, 159 bp, Artificial Sequence
  Fusion sequence
ggagacctacaaactgaagtgcaaggcactgcaggaggagaaccgcgacctgcgcaaagccagcgtgaccatctttgcccacaagccacccatctcctcagctgagatgaccttccggaggcccgcccaggccttcccggtcagctactcctcttccgg

SEQ ID NO: 173 - DNA, 126 bp, Artificial Sequence
  Fusion sequence
aggagaaagaaacccttgctgtaaattatgagaaagaagaagaattcctcactaatgagctctccagaaaattgatgcagatccactgtgcgacgagctgtgccgcacggtgatcgcagccgctgt

SEQ ID NO: 174 - DNA, 146 bp, Artificial Sequence
  Fusion sequence
cggctgaagaagcaactgagagctgctcagttacagcagtcttgctgtgttgcccacaagtttgcccacaagccacccatctcctcagctgagatgaccttccggaggcccgcccaggccttcccggtcagctactcctcttccgg

SEQ ID NO: 175 - DNA, 119 bp, Artificial Sequence
  Fusion sequence
ggagacctacaaactgaagtgcaaggcactgcaggaggagaaccgcgacctgcgcaaagccagcgtgaccatcggatcaccaggaacttctccacctgctctcccagcaccaagacctg

SEQ ID NO: 176 - DNA, 119 bp, Artificial Sequence
  Fusion sequence
cggctgaagaagcaactgagagctgctcagttacagctctggcatagaagattaaagaatcaaaaaagtgccaaggaaggggtgacagtgcttataaacgaagacaaagagttggctga

SEQ ID NO: 177 - DNA, 132 bp, Artificial Sequence
  Fusion sequence
cttacacaccttctccgagttcaagcaggcctatatcacctgccttgccctttaaggtggtggtgatctcagccatcctggccctggtggtgctcaccatcatctcccttatcatcctcatcatgctttggc

SEQ ID NO: 178 - DNA, 155 bp, Artificial Sequence
  Fusion sequence
ctcccagagaccaacgttcaagcagttggtagaagacttggatcgaattctcactctcacaaccaatgagcaagccagggctgagcaggaagaagaattcattagtaacactttattcaagaaaattcaggctttgcagaaggagaaagaaaccc

SEQ ID NO: 179 - DNA, 164 bp, Artificial Sequence
  Fusion sequence
agaaagtaccaatcagaaggacgtgttgcttcaggcctggagccctcccttctcaaagagaaccctgcgggcaacttatgactcaagatgggagtttccaagagatggactagtgcttggtcgggtcttggggtctggagcgtttgggaaggtggttgaaggaa

SEQ ID NO: 180 - DNA, 147 bp, Artificial Sequence
  Fusion sequence
ctcccagagaccaacgttcaagcagttggtagaagacttggatcgaattctcactctcacaaccaatgaggcacatagagatgaaatccagcgcaaatttgatgctcttcgtaacagctgtactgtaatcacagacctggaggagca

SEQ ID NO: 181 - DNA, 138 bp, Artificial Sequence
  Fusion sequence
ggaggaaggtctgaggagcagcttcagtccccgccgagccgccaccgcaggtcgaggacggtcggactcccgcggcgggaggagcctgttcccctgaggtttcggctgagtccagctcctccatgaactccaacaccc

SEQ ID NO: 182 - DNA, 91 bp, Artificial Sequence
  Fusion sequence
tcttgcagcagaacccagttactgaatatatgaaaaaggacttgattagagaccaaggatttcgtggtgatggaggatcaaccacaggttt

SEQ ID NO: 183 - DNA, 157 bp, Artificial Sequence
  Fusion sequence
ccagtcgtcagaccccagacctccccacctcccacctgcaccagcaccaacgggaacagcctgcaagccttgccctttaaggtggtggtgatctcagccatcctggccctggtggtgctcaccatcatctcccttatcatcctcatcatgctttggc

SEQ ID NO: 184 - DNA, 164 bp, Artificial Sequence
  Fusion sequence
ccatcgattatggtttccagaggttacagaaggtcattcctcggcaccctggtgaccctgagcgtttgccaaagccttgccctttaaggtggtggtgatctcagccatcctggccctggtggtgctcaccatcatctcccttatcatcctcatcatgctttggc

SEQ ID NO: 185 - DNA, 164 bp, Artificial Sequence
  Fusion sequence
ctctgccgcaatgtccaatttgggcggctcccccaccttcctcaacggctcagctgccaactccccctatgccaccttgccctttaaggtggtggtgatctcagccatcctggccctggtggtgctcaccatcatctcccttatcatcctcatcatgctttggc

SEQ ID NO: 186 - DNA, 162 bp, Artificial Sequence
  Fusion sequence
ctctgccgcaatgtccaatttgggcggctcccccaccttcctcaacggctcagctgccaactccccctatgccattcttcaggagagaataccatgggtaccacctgaatgcattgaaaatcctaaaaatttaaatttggcaacagacaaatggagttttgg

SEQ ID NO: 187 - DNA, 139 bp, Artificial Sequence
  Fusion sequence
ctcgcatcgcgcactttttggatcggcatctagtctttccgcttcttgaatttctctctgtaaaggaggttcgtggcggagagatgctgatcgcgctgaactgaccggtgcggcccgggggtgagtggcgagtctccct

SEQ ID NO: 188 - DNA, 150 bp, Artificial Sequence
  Fusion sequence
ctcgcatcgcgcactttttggatcggcatctagtctttccgcttcttgaatttctctctgtaaaggagctagttatgtatcaaatcccatttgcaagggttgtttgtcttgttcaaaggacaatgggtgtagccgatgtcaacagaagtt

SEQ ID NO: 189 - DNA, 139 bp, Artificial Sequence
  Fusion sequence
ctgcaacctctgcctccttagttcaagcgattctcctgcctcagcctcctgagtagctggtactacaggttcgtggcggagagatgctgatcgcgctgaactgaccggtgcggcccgggggtgagtggcgagtctccct

SEQ ID NO: 190 - DNA, 155 bp, Artificial Sequence
  Fusion sequence
aaaactgggagagcttcggaaaaagcactacgagcttgctggtgttgctgagggctgggaagaagccttgccctttaaggtggtggtgatctcagccatcctggccctggtggtgctcaccatcatctcccttatcatcctcatcatgctttggc

SEQ ID NO: 191 - DNA, 144 bp, Artificial Sequence
  Fusion sequence
ctaaagcagctcaaaagaaagcagaagccactggagaaaaacggccaagaggcagacctaggaaatgggttatcttgacgaatcagattacaacccatctgagtggagccctggcttctcaggcagacctggtgtctccagctg

SEQ ID NO: 192 - DNA, 107 bp, Artificial Sequence
  Fusion sequence
ggcccttcgagagcaagtttaagaaggagccggccctgactgcagacactaacagcacatctggagacccggtggagaagaaggacgaaacaccttttggggtctcg

SEQ ID NO: 193 - DNA, 171 bp, Artificial Sequence
  Fusion sequence
tgacctgcgcatgtctgccatggcccccacaccgccccagggtgaggttgacgccgactgcatggacgtcaatgtccgcgggcctgccggacccagcaggacgggatatctccatccgccctctcctggagcactgtgagaacacccatatgaccatctggcttggcatcg

SEQ ID NO: 194 - DNA, 115 bp, Artificial Sequence
  Fusion sequence
ctgaggtgtctgtgctcgtcgccagcgtcgggtgggctttcgcccgcggctcctgagggatcggtctcagccgcgcggctccatcgtctacctggagattgacaaccggcagtgt

SEQ ID NO: 195 - DNA, 133 bp, Artificial Sequence
  Fusion sequence
ctgaggtgtctgtgctcgtcgccagcgtcgggtgggctttcgcccgcggctcctgagggatcggtctcagccgcgcgggtgagaccgtggagccgcccccgccggcgcagctgcacttcatgtacgtggcggc

SEQ ID NO: 196 - DNA, 146 bp, Artificial Sequence
  Fusion sequence
gctgggtctaattagttgagaagcagtgacacccccaaccactccccaaacaggctggctcccgtctccaggccccaaggagccacacctggaccagaccccaggaaagctccatcgtctacctggagattgacaaccggcagtgt

SEQ ID NO: 197 - DNA, 150 bp, Artificial Sequence
  Fusion sequence
cggtgagacctgcctgaatggcgggaagtgtgaagcggccaatggcacggaggcctgcgtttcttcagtgccctactccacagccaaaacacctcacccagtgttgaccccagtggctgctaaccaagccaagcaggggtctctaataaa

SEQ ID NO: 198 - DNA, 132 bp, Artificial Sequence
  Fusion sequence
actgtgaggacctggtggacgagtgctcacccagcccctgccagaacggggccacctgcacggactacctgggcggctactcctgcaagctgaaagatgaaaattctaagctgagaagaaagctgaatgagg

SEQ ID NO: 199 - DNA, 149 bp, Artificial Sequence
  Fusion sequence
cggtgagacctgcctgaatggcgggaagtgtgaagcggccaatggcacggaggcctgcgtatgcagaggccaggatgtgggcccagccctgtgccaggaggctggctggaataaagagtaacaaacccccttggaggactctcctgccg

SEQ ID NO: 200 - DNA, 120 bp, Artificial Sequence
  Fusion sequence
ctcccagagaccaacgttcaagcagttggtagaagacttggatcgaattctcactctcacaaccaatgagacacaacttcgaaaccagctaattcatgagttgatgcaccctgtattgag

SEQ ID NO: 201 - DNA, 143 bp, Artificial Sequence
  Fusion sequence
aatctgctcacagtgaaaatcctttagagaaatacatgaaaatcatccagcaggagcaagaccaggagtcggcagataagaatgaaagccttggccaaggcacttttacaaagatttttaaaggcgtacgaagagaagtagga

SEQ ID NO: 202 - DNA, 129 bp, Artificial Sequence
  Fusion sequence
ccctcacagagacccaccttcaagcagctggtggaagacctggaccgcatcgtggccttgacctccaaccaggggctgctggagtcctctgcagagaaggcccctgtgtcggtgtcctgtggaggtgag

SEQ ID NO: 203 - DNA, 110 bp, Artificial Sequence
  Fusion sequence
tccgtccctgtcccctttcctgctggcaggagccggctgcctaccaggggcctgggctgctggagtcctctgcagagaaggcccctgtgtcggtgtcctgtggaggtgag

SEQ ID NO: 204 - DNA, 159 bp, Artificial Sequence
  Fusion sequence
ctcccagagaccaacgttcaagcagttggtagaagacttggatcgaattctcactctcacaaccaatgaggtaaaggcgacacaggaggagaaccgggagctgaggagcaggtgtgaggagctccacgggaagaacctggaactggggaagatcatgga

SEQ ID NO: 205 - DNA, 115 bp, Artificial Sequence
  Fusion sequence
ggacctggaccgtgtccttaccgtgacgtccaccgacgtgccaggcccacccccaggtgttcccgcgcctgggggcccacccctgtccaccggacctatagtggacctgctccag

SEQ ID NO: 206 - DNA, 114 bp, Artificial Sequence
  Fusion sequence
ggacctggaccgtgtccttaccgtgacgtccaccgactttaaggagtcggccttgaggaagcagtccttatacctcaagttcgaccccctcctgagggacagtcctggtagacc

SEQ ID NO: 207 - DNA, 126 bp, Artificial Sequence
  Fusion sequence
ggacctggaccgtgtccttaccgtgacgtccaccgacgtaaaggcgacacaggaggagaaccgggagctgaggagcaggtgtgaggagctccacgggaagaacctggaactggggaagatcatgga

SEQ ID NO: 208 - DNA, 128 bp, Artificial Sequence
  Fusion sequence
ggccttgtttgaccgagtctacactcaccagagtgacgtgtaaaggcgacacaggaggagaaccgggagctgaggagcaggtgtgaggagctccacgggaagaacctggaactggggaagatcatgga

SEQ ID NO: 209 - DNA, 144 bp, Artificial Sequence
  Fusion sequence
ggagctcttcaagctgctgaaggagggccaccgcatggacaagcccgccaactgcacacacgacctgtgccaggcccacccccaggtgttcccgcgcctgggggcccacccctgtccaccggacctatagtggacctgctccag

SEQ ID NO: 210 - DNA, 155 bp, Artificial Sequence
  Fusion sequence
ggagctcttcaagctgctgaaggagggccaccgcatggacaagcccgccaactgcacacacgacctgtaaaggcgacacaggaggagaaccgggagctgaggagcaggtgtgaggagctccacgggaagaacctggaactggggaagatcatgga

SEQ ID NO: 211 - DNA, 148 bp, Artificial Sequence
  Fusion sequence
gcacacacgacctgtacatgatcatgcgggagtgctggcatgccgcgccctcccagaggcccaccttcaagcagaaggaactttccaaagctgaaatccagaaagttctaaaagaaaaagaccaacttaccacagatctgaactccat

SEQ ID NO: 212 - DNA, 124 bp, Artificial Sequence
  Fusion sequence
ggacctggaccgtgtccttaccgtgacgtccaccgacgcagctgcattcagcctcagcggaggacacgcctgtggtgcagttggcagccgagaccccaacagcagagagcaaggagagagcctt

SEQ ID NO: 213 - DNA, 113 bp, Artificial Sequence
  Fusion sequence
ggacctggaccgtgtccttaccgtgacgtccaccgacgagagagccttgaactctgccagcacctcgcttcccacaagctgtccaggcagtgagccagtgcccacccatcagc

SEQ ID NO: 214 - DNA, 123 bp, Artificial Sequence
  Fusion sequence
ggacctggaccgtgtccttaccgtgacgtccaccgaccatgcacggtgcaaatgagactccctcaggacgtccgcgggaagccaagcttgtggagttcgatttcttgggagcactggacattc

SEQ ID NO: 215 - DNA, 144 bp, Artificial Sequence
  Fusion sequence
ggacctggaccgtgtccttaccgtgacgtccaccgacgagtacctggagcagtttggaacttcctcgtttaaggagtcggccttgaggaagcagtccttatacctcaagttcgaccccctcctgagggacagtcctggtagacc

SEQ ID NO: 216 - DNA, 155 bp, Artificial Sequence
  Fusion sequence
gtgaccgaggacaacgtgatgaagatcgcagacttcgggctggcccgggacgtgcacaacctcgacgtaaaggcgacacaggaggagaaccgggagctgaggagcaggtgtgaggagctccacgggaagaacctggaactggggaagatcatgga

SEQ ID NO: 217 - DNA, 117 bp, Artificial Sequence
  Fusion sequence
ggcgcctttcgagcagtactccccgagccagcagctgcattcagcctcagcggaggacacgcctgtggtgcagttggcagccgagaccccaacagcagagagcaaggagagagcctt

SEQ ID NO: 218 - DNA, 163 bp, Artificial Sequence
  Fusion sequence
ggcgcctttcgagcagtactccccgggtggccaggacacccccagctccagctcctcaggggacgactccgtgtttgcccacgacgtgccaggcccacccccaggtgttcccgcgcctgggggcccacccctgtccaccggacctatagtggacctgctccag

SEQ ID NO: 219 - DNA, 171 bp, Artificial Sequence
  Fusion sequence
ggcgcctttcgagcagtactccccgggtggccaggacacccccagctccagctcctcaggggacgaggacctggatgcagtggtaaaggcgacacaggaggagaaccgggagctgaggagcaggtgtgaggagctccacgggaagaacctggaactggggaagatcatgga

SEQ ID NO: 220 - DNA, 117 bp, Artificial Sequence
  Fusion sequence
ggcgcctttcgagcagtactccccgggtgtaaaggcgacacaggaggagaaccgggagctgaggagcaggtgtgaggagctccacgggaagaacctggaactggggaagatcatgga

SEQ ID NO: 221 - DNA, 92 bp, Artificial Sequence
  Fusion sequence
ggacctggaccgtgtccttaccgtgaatggaattctacagaaaccagtggaggctgacaccgacctcctgggggatgcaagcccagcctttg

SEQ ID NO: 222 - DNA, 187 bp, Artificial Sequence
  Fusion sequence
gcacacacgacctgtacatgatcatgcgggagtgctggcatgccgcgccctcccagaggcccaccttcaagcagctggtggagtacctggagcagtttggaacttcctcgtttaaggagtcggccttgaggaagcagtccttatacctcaagttcgaccccctcctgagggacagtcctggtagacc

SEQ ID NO: 223 - DNA, 187 bp, Artificial Sequence
  Fusion sequence
gcacacacgacctgtacatgatcatgcgggagtgctggcatgccgcgccctcccagaggcccaccttcaagcagctggtggaggacctggatgcagtggtaaaggcgacacaggaggagaaccgggagctgaggagcaggtgtgaggagctccacgggaagaacctggaactggggaagatcatgga

SEQ ID NO: 224 - DNA, 124 bp, Artificial Sequence
  Fusion sequence
gcacacacgacctgtacatgatcatgcgggagtgctggcatgccgcgccctcccagaggcccaccttcaagcagctggtggaggacctccacgggaagaacctggaactggggaagatcatgga

SEQ ID NO: 225 - DNA, 125 bp, Artificial Sequence
  Fusion sequence
gcacacacgacctgtacatgatcatgcgggagtgctggcatgccgcgccctcccagaggcccaccttcaagcagcaggtgtgaggagctccacgggaagaacctggaactggggaagatcatgga

SEQ ID NO: 226 - DNA, 137 bp, Artificial Sequence
  Fusion sequence
ggtgccacccgcctatgcccctccccctgccgtccccggccatcctgccccccagagtgctgaggtgtggggcgggccttctggcccaggtgccctggctgacctggactgctcaagctcttcccagagcccaggaa

SEQ ID NO: 227 - DNA, 107 bp, Artificial Sequence
  Fusion sequence
ggtgccacccgcctatgcccctccccctgccgtccccggccatccctcaggacgtccgcgggaagccaagcttgtggagttcgatttcttgggagcactggacattc

SEQ ID NO: 228 - DNA, 121 bp, Artificial Sequence
  Fusion sequence
ggacctggaccgtgtccttaccgtgacgtccaccgacaacgaagagtcactgaagaagtgcgtggaggattacctggcaaggatcacccaggagggccagaggtaccaagccctgaaggcc

SEQ ID NO: 229 - DNA, 138 bp, Artificial Sequence
  Fusion sequence
ggacctggaccgtgtccttaccgtgacgtccaccgacgagtacctggacctgtcggcgacacaggaggagaaccgggagctgaggagcaggtgtgaggagctccacgggaagaacctggaactggggaagatcatgga

SEQ ID NO: 230 - DNA, 220 bp, Artificial Sequence
  Fusion sequence
ggcgcctttcgagcagtactccccgggtggccaggacacccccagctccagctcctcaggggacgactccggaggtcctgggagggtcagtctggcccgcctgcctgctgacttgggtgtggcctgagcaggtaaaggcgacacaggaggagaaccgggagctgaggagcaggtgtgaggagctccacgggaagaacctggaactggggaagatcatgga

SEQ ID NO: 231 - DNA, 188 bp, Artificial Sequence
  Fusion sequence
gcacacacgacctgtacatgatcatgcgggagtgctggcatgccgcgccctcccagaggcccaccttcaagcagctggtggaaggacctggatgcagtggtaaaggcgacacaggaggagaaccgggagctgaggagcaggtgtgaggagctccacgggaagaacctggaactggggaagatcatgga

SEQ ID NO: 232 - DNA, 151 bp, Artificial Sequence
  Fusion sequence
ggacctggaccgtgtccttaccgtgacgtccaccgactgaaggcccacgcggaggagaagctgcagctggcaaacgaggagatcgcccaggtccggagcaaggcccaggcggaagcgttggccctccaggccagcctgaggaaggagcaga

SEQ ID NO: 233 - DNA, 147 bp, Artificial Sequence
  Fusion sequence
ggtgccacccgcctatgcccctccccctgccacggaggagccaggtccctgtctgagccagcagctgcattcagcctcagcggaggacacgcctgtggtgcagttggcagccgagaccccaacagcagagagcaaggagagagcctt

SEQ ID NO: 234 - DNA, 177 bp, Artificial Sequence
  Fusion sequence
ccaaagtacccaaaggcaagtggaacagctctaatggggttgaagaaaaggagacttgggtggaagaggatgaactgtttcaagttcaggattatgaactattaacagaaaatgacatgttaccaaatatgaggataggtgccctggggttttctggtgcctttgaagaccgggatc

SEQ ID NO: 235 - DNA, 186 bp, Artificial Sequence
  Fusion sequence
agatggaagaaatggagatgtctctcagtttccatatgtggaatttacaggaagagatagtgtcacctgccctacttgtcagggaacaggaagaattcctagggtctggcatagaagattaaagaatcaaaaaagtgccaaggaaggggtgacagtgcttataaacgaagacaaagagttggctga

SEQ ID NO: 236 - DNA, 147 bp, Artificial Sequence
  Fusion sequence
caggtctgtctgccagtaattagtgccgaaaactggaagccagcaaccaaaaccgaccaaggcctgctgaaaatgactgaatataaacttgtggtagttggagctggtggcgtaggcaagagtgccttgacgatacagctaattcag

SEQ ID NO: 237 - DNA, 140 bp, Artificial Sequence
  Fusion sequence
ctcccagagaccaacgttcaagcagttggtagaagacttggatcgaattctcactctcaagttgctggagaatgtaaccctaatccataaaccagtgtcgttgcaaccccgtgggctgatcaataaagggaactggtgct

SEQ ID NO: 238 - DNA, 121 bp, Artificial Sequence
  Fusion sequence
ctgcaatttgggttgcaacaactaagtctacagtaaataaatggaagccacgttacgagatccgatggaaggtgattgagtctgtgagctctgacggccatgagtacatctacgtggaccc

SEQ ID NO: 239 - DNA, 150 bp, Artificial Sequence
  Fusion sequence
gcttactgaccaacctggcagacagggagctggttcacatgatcaactgggcgaagagggtgccaggtcctcttcctgatggatgggaacaagccatgactcaggatggagaaatttactatataaaccataagaacaagaccacctctt

SEQ ID NO: 240 - DNA, 146 bp, Artificial Sequence
  Fusion sequence
cagagagtggcatgtatgcatgtgacttatgtgacaagacattccagaaaagcagttcccttctgcgacataaatacgaacacacagtccctgtccgagtgctggagctaagtgagagccaccctgacagtggggaacagacagtc

SEQ ID NO: 241 - DNA, 144 bp, Artificial Sequence
  Fusion sequence
gctcgacccttgacctttctggctccagagagacgccctcctccattctcttaggctccaaccaaggctctgaactgttccccctcatcttcccggcagagccagcccaggcctctggcccctatgtggagatcattgagcagc

SEQ ID NO: 242 - DNA, 19 bp, Artificial Sequence
  Forward Primer
cccagttctgcgcagcttc

SEQ ID NO: 243 - DNA, 45 bp, Artificial Sequence
  Insert Sequence
ccgaggctccgcaccagccgcgcttctgtccgcctgcagggcatt

SEQ ID NO: 244 - DNA, 27 bp, Artificial Sequence
  Reverse Primer
agacagcaaatatcctcatctttctgg

SEQ ID NO: 245 - DNA, 28 bp, Artificial Sequence
  Forward Primer
tgctgtctttatattcatgacctactgg

SEQ ID NO: 246 - DNA, 61 bp, Artificial Sequence
  Insert Sequence
catttgctgaacgcatttactgtcacggttcccaaggacctatatgtggtagagtatggta

SEQ ID NO: 247 - DNA, 28 bp, Artificial Sequence
  Reverse Primer
gggaatttgcattcaattgtcatattgc

SEQ ID NO: 248 - DNA, 21 bp, Artificial Sequence
  Forward Primer
cgctgcatgatcagctatggt

SEQ ID NO: 249 - DNA, 76 bp, Artificial Sequence
  Insert Sequence
ggtgccgactacaagcgaattactgtgaaagtcaatgccccatacaacaaaatcaaccaaagaattttggttgtgg

SEQ ID NO: 250 - DNA, 23 bp, Artificial Sequence
  Reverse Primer
ttcatgttcagaggtgactggat

SEQ ID NO: 251 - DNA, 27 bp, Artificial Sequence
  Forward Primer
actgcacttttaggagattagatcctg

SEQ ID NO: 252 - DNA, 70 bp, Artificial Sequence
  Insert Sequence
aggaaaaccatacagctgaattggtcatcccagaactacctctggcacatcctccaaatgaaaggactca

SEQ ID NO: 253 - DNA, 23 bp, Artificial Sequence
  Reverse Primer
agatggctcccagaattaccaag

SEQ ID NO: 254 - DNA, 22 bp, Artificial Sequence
  Forward Primer
agcactgacattcatcttccgt

SEQ ID NO: 255 - DNA, 75 bp, Artificial Sequence
  Insert Sequence
ttaagaaaagggagaatgatggatgtgaaaaaatgtggcatccaagatacaaactcaaagaagcaaagtgataca

SEQ ID NO: 256 - DNA, 23 bp, Artificial Sequence
  Reverse Primer
ctggattacgtctcctccaaatg

(19) United States
(12) Patent Application Publication
(10) Pub. No.: US 2024/0006024 A1
(43) Pub. Date: Jan. 4, 2024

(54) PD-L1 EXPRESSION AS MARKER FOR CANCER TREATMENT RESPONSE

(71) Applicant: ONCOLOGICA UK LIMITED, Cambridge (GB)

(72) Inventors: Marco LODDO, Cambridge (GB); Gareth WILLIAMS, Cambridge (GB); Keeda HARDISTY, Cambridge (GB)

(73) Assignee: ONCOLOGICA UK LIMITED, Cambridge (GB)

(21) Appl. No.: 17/998,784

(22) PCT Filed: May 14, 2021
(86) PCT No.: PCT/GB2021/051176

§ 371 Date: Nov. 14, 2022

(30) Foreign Application Priority Data
May 15, 2020 (GB) 2007218.7

(51) Int. Cl. G16B 25/10; G16H 20/00

(52) U.S. Cl. CPC G16B 25/10; G16H 20/00

(57) ABSTRACT

A method for determining the susceptibility of a patient suffering from proliferative disease to treatment using an agent targeting a cell pathway or components thereof comprises an immune-checkpoint comprising components of the PD1/PD-L1 pathway, an agent targeting DDR signalling pathway comprising PARP inhibitors, DDR inhibitors and cell cycle checkpoint inhibitors, or a combination of thereof. The method comprises determining tumour type, determining expression levels of PD-L1, determining tumour mutational burden, preparing a DNA damage and repair related genes analysis based on the tumour type and PD-L1 expression levels.

The present invention relates to a method for determining the susceptibility of a patient suffering from proliferative disease, such as cancer, to treatment using a target agent. It further comprises the development of treatment regimens for selected patients, based upon the determination, and computers programmed to carry out the determination.

### BACKGROUND OF THE INVENTION

Programmed death 1 receptor (PD-1) and its ligands, PD-1 programmed death-ligand 1 (PD-L1) and PD-L2, deliver inhibitory signals that regulate the balance between T cell activation, tolerance, and immunopathology. The PD-L1 is a transmembrane protein that binds to the PD-1 during immune system modulation. This PD-1/PD-L1 interaction protects normal cells from immune recognition by inhibiting the action of T-cells thereby preventing immune-mediated tissue damage. The PD-1/PD-L1 pathway is normally involved in promoting tolerance and preventing tissue damage in the setting of chronic inflammation.

Harnessing the immune system in the fight against cancer has become a major topic of interest. Immunotherapy for the treatment of cancer is a rapidly evolving field from therapies that globally and non-specifically stimulate the immune system to more targeted approaches.

The PD-1/PD-L1 pathway has emerged as a powerful target for immunotherapy. A range of cancer types have been shown to express PD-L1 which binds to PD-1 expressed by immune cells resulting in immunosuppressive effects that allows these cancers to evade tumour destruction. The PD-1/PD-L1 interaction inhibits T-cell activation and augments the proliferation of T-regulatory cells (T-regs) which further suppresses the effector immune response against the tumour. This mimicks the approach used by normal cells to avoid immune recognition. Targeting PD-1/PD-L1 has therefore emerged as a new and powerful approach for immunotherapy directed therapies.

Disrupting the PD-1/PD-L1 pathway with therapeutic antibodies directed against either PD-1 or PD-L1 (anti-PD-L1 or anti-PD-1 agents) results in restoration of effector immune responses with preferential activation of T-cells directed against the tumour.

All solid tumours and haematological malignancies including, melanoma, renal cell carcinoma, lung cancers of the head and neck, gastrointestinal tract malignancies, ovarian cancer, haematological malignancies are known to express PD-L1 resulting in immune evasion. Anti-PD-L1 and anti-PD-1 therapy has been shown to induce a strong clinical response in many of these tumour types, for example 20-40% in melanoma and 33-50% in advanced non-small cell lung cancer (NSCLC). A number of these antibodies, for example anti-PD-1 directed agents Nivolumab and Pembrolizumab, have now received FDA-approval for the treatment of metastatic NSCLC and advanced melanoma.

There are nine drugs in development targeting the PD-1/PD-L1 pathway, and the current practice of pharmaceutical companies is to independently develop an anti-PD-L1 IHC diagnostic assays as a predictor of response to anti PD-1/anti PD-L1 directed therapies. These PD-1/PD-L1 directed therapies include Pembrolizumab, atezolizumab, avelumab, nivolumab, durvalumab, PDR-001, BGB-A317, REG W2810 and SHR-1210.

The leading Biopharma companies have all chosen an immunohistochemical approach on paraffin wax embedded formalin fixed diagnostic biopsies and resection tissues/samples (PWET) for the development of companion diagnostics for anti-PD-1/PD-L1 directed therapies. All these tests involve the application of a monoclonal antibody raised against PD-L1 applied to the tissue section using a standard immunohistochemical assay approach with enzyme linked chromogen detection systems. The immunohistochemical staining of cells, either partial or complete surface membrane staining for PD-L, is then assessed manually by microscopic examination by a pathologists to determine the proportion of cells which express PD-L1. A tumour proportion score is then reported. Some assays assess only the tumour cell expression of PD-L1, others assess both tumour cells and the expression of PD-L1 in the associated intratumoural and peritumoural immune cell infiltrates (ICs).

Several independently developed PD-L1 immunohistochemical (IHC) predictive assays are commercially available. Published studies using the VENTANA PD-L1 (SP263) Assay, VENTANA PD-L1 (SP142) Assay, Dako PD-L1 IHC 22C3 pharmDx assay, Dako PD-L1 IHC 28-8 pharmDx assay, and laboratory-developed tests utilizing the E1L3N antibody (Cell Signaling Technology), have demonstrated differing levels of PD-L1 staining between assays. Moreover, different cut-points have been developed for prediction of response in relation to the tumour proportion score and/or PD-L1 positive IC populations.

However major problems have arisen in relation to the ability of these IHC PD-L1 companion diagnostic assays to predict response to anti-PD-L1/PD-1 directed therapies.

For instance, it has been observed that the percentage of PD-L1-stained tumour cells varies with the type of IHC assay used. For example, comparable results are observed in relation to 22C3, 28-8, and SP263 whereas the SP142 assay exhibits fewer stained tumour cells.

PD-L1 ring studies have also shown poor correlation between the scores generated by individual pathologists. The poor Inter-reader reliability is a particular problem in the assessment of PD-L1 immune cell populations.

The immune checkpoint involves not only PD-L1 but many other biological factors. For example, the PD-L1 signalling axis involves other major components in addition to PD-1 and PD-L1 which have been shown to be predictors of response to anti-PD-1/PD-L1/PD-L2 directed immunotherapy agents including aberration of NFATC1, PIK3CA, PIK3CD, PRDM1, PTEN, PTPN11, MTOR, HIF1A, FOX01.

Similar issues arise with regard to tests developed for drugs developed to target other cell pathways or components thereof such as DDR/MMR signalling pathway.

Accordingly, there is a need to develop further methods to determine the susceptibility of a patient suffering from proliferative disease, such as cancer, to treatment using particular types of agent.

### SUMMARY OF THE INVENTION

According to the present invention there is provided a method for determining the susceptibility of a patient suffering from proliferative disease to treatment using an agent targeting a cell pathway or components thereof comprising an immune-checkpoint comprising components of the PD1/PD-L1 pathway, an agent targeting DDR/MMR signalling pathway comprising PARP inhibitors, DDR inhibitors and cell cycle checkpoint inhibitors, or a combination of thereof, said method comprising determining tumour type, determining expression levels of PD-L1, determining tumour mutational burden, preparing a DNA damage and repair related genes analysis based on the tumour type and the PD-L1 expression levels.

PD-L1 mRNA expression levels can be measured using next generation sequencing (NGS) analysis to provide a readout measured in RPM (Reads per million mapped reads). The RPM reads were first normalised and a log score generated to derive a nLRPM.

It has been identified that the pattern of DNA damage and repair related (DDR) genes within a cell is dependent upon the tumour type and the PD-L1 expression of the cell. Therefore, instead of having to conduct a scattergun approach to the analysis of DDR genes within tumour cells a targeted approach can be followed. This allows the analysis to be carried out more efficiently and effectively. Further, if the PD-L1 expression levels are 10% or greater (i.e. 7 or more nLRPM) then fewer DDR genes will need to be investigated. Accordingly, although this is a complex and multicomponent system, it provides a simple approach.

The present method can be used in relation to treatments using an agent which targets immune checkpoint components, for example, the PD-L1 signalling axis, Wnt/β-catenin, RAS/RAF/MEK/ERK, PI3K/AKT/MTOR, TGF-β, ID01 and JAK/STAT signaling pathways, TMB-neoantigen load and HLA variability and pathways involved in innate and adaptive immune responses, druggable immune checkpoint components, for example, PD-1/PD-L1, CTLA-4, B7-1 and B7-2, and druggable targets in the DNA damage and response (DDR) signaling pathways include, for example, PARP, DNA-PK, Cdc7, ATM, ATR, CHK1 and CHK2.

Agents which target immune checkpoint components include Pembrolizumab, atezolizumab, avelumab, nivolumab, durvalumab, PDR-001, BGB-A317, REG W2810, SHR-1210 against PD-1/PD-L1 and Ipilimumab, Tremelimumab against CTLA-4. PARP can be targeted by agents such as rucaparib, veliparib, niraparib, DNA-PK by agents such as omipalisib, DMNB, compound 401, AZD7648, Cdc7 by agents such as LY3143921 or SRA141, ATM by agents such as AZD0156, ATR by agents such as AZD6738 and BAY 1895344, CHK1 by agents such as prexasertib and SRA737, CHK2 by agents such as CCT241533 and LY2606368.

Further, the present approach can be used when a combination of agents, such as those aforementioned, are being used.

In the present invention, analysis of the tumour mutational burden (TMB) can take place at any point of time in the method of the present invention.

The analysis of the TMB is not specific to the tumour type nor the PD-L1 expression levels and, therefore, can be conducted at any stage of the method. Determining the levels of TMB is a well known practice and many methods will be known to those skilled in the art.

Conveniently testing is performed on formalin fixed paraffin wax embedded tissue samples (PWET). Quantative analysis of RNA performed in parallel and integrated with DNA DDR mutation analysis has to date been a technical challenge because formalin fixation results in degradation of nucleic acid resulting in low DNA/RNA yields with low integrity and quality. In the present invention, the combined PD-L1-DDR NGS assay design is unique in being able to analyse PWET tissues and circumvent the problem of degraded DNA/RNA thereby enabling a combined integrated PD-L1 mRNA gene expression and DDR signature to be generated.

Conveniently the tumour type is selected from bladder, breast, cervical, colorectal, cancer of unknown primary (CUP), endometrial, gallbladder, gastric, glioblastoma, glioma, gastro oesophageal junction, head and neck, kidney, liver, lung, melanoma, mesothelioma, oesophageal, ovarian, pancreatic, prostrate, sarcoma, small bowel and thyroid. Tumours of other origins can also be included under the term “Other”. In this regard, the DDR analysis of some “Other” cancers have been identified in Table A. However, it will be appreciated that many “Other” cancers may not be encompassed by the DDR analysis. However, the experimental protocol in the present application allows a person skilled in the art to carry out the relevant analysis of the tumour to identify the DDR genes which would be relevant for analysis in the relevant cancer.

The tumour is typed by any method known to those skilled in the art. Tumour typing is a well known practice and many methods will be known to those skilled in the art.

The tumour type is based upon the origin of the cancer and not the tissue type. In this regard, it will be appreciated by those skilled in the art that, for example, breast cancer can spread to bones, liver, lungs and/or brain. However, despite not being in the breast the tumour type will remain breast cancer.

Conveniently the DNA damage and repair (DDR) related gene analysis is prepared using the tumour type and PDL-1 gene expression levels to select the core genes in Table A for analysis.

DDR genes analysis is a well known practice and many methods will be known to those skilled in the art.

It has been found that the presence of specific DDR genes is dependent upon the tumour type and the PD-L1 expression levels. Table A sets out the core DDR genes which should be investigated for specific tumour types. Other DDR genes could also be analysed.

Conveniently scores are assigned to each of the analysed parameters:
i) a score of ‘0’ is applied in the absence of PD-L1 expression;
ii) a score of ‘1’ is applied in the presence <7 nLRPM but not 0 in relation to PD-L1 expression;
iii) a score of ‘2’ is applied in the presence 7-10 nLRPM in relation to PD-L1 expression;
iv) a score of ‘3’ is applied in the presence >10 nLRPM in relation to PD-L1 expression;
v) a score of ‘0’ is applied if the tumour mutational burden is ‘low’;
vi) a score of ‘1’ is applied if the tumour mutational burden is ‘high’;
vii) a score of ‘0’ is applied if there are no aberrations in the DNA damage and repair related genes analysis;
viii) a score of ‘1’ is applied if there is 1 aberration in the DNA damage and repair related genes analysis;
xi) a score of ‘2’ is applied if there are 2 aberrations in the DNA damage and repair related genes analysis;
x) a score of ‘3’ is applied if there are aberrations in the DNA damage and repair related genes analysis;
wherein an overall score of 0 is indicative of no susceptibility to the target agent, an overall score of 1-2 indicates a weak response, an overall score of 3-4 indicates a moderate response, and an overall score of 5 to 7 indicates a strong response.

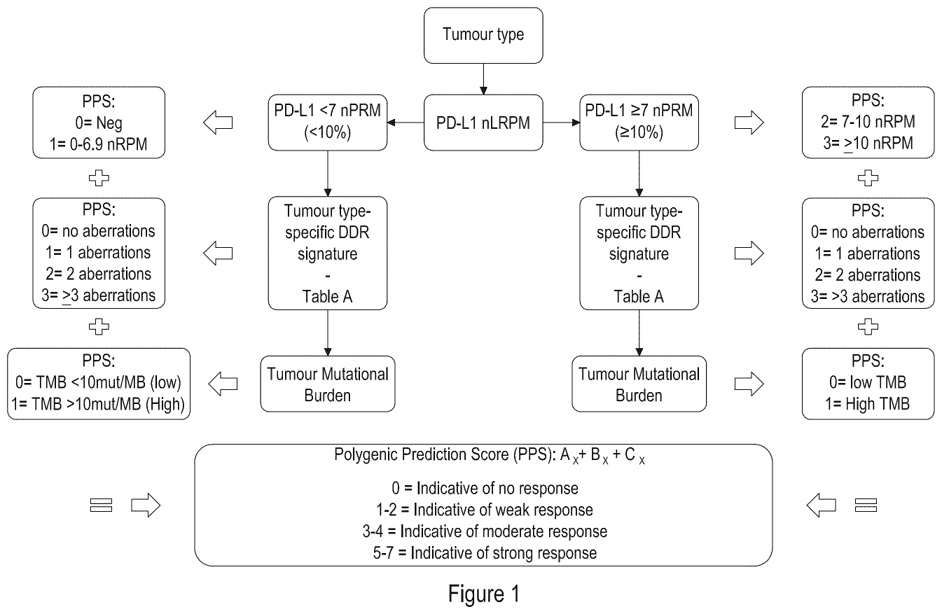

An example of this method is illustrated in FIG. 1.

This scoring system ensures that there is less likelihood of poor inter-reader reliability. The scores given are based on absolute values. Further, it allows a complex, multicomponent predictive system to be utilised but in a simple manner.

If a moderate or strong response is shown then the relevant practitioner has empirical data to support starting or continuing the patient on a certain treatment. Further if a weak or null response is given then alternative treatments can be explored at an early stage which can be vital when treating proliferative diseases such as cancer.

Conveniently the tumour mutational burden is designated ‘low’ if there are <10 mut/MB and the tumour mutational burden is designated ‘high’ if there are ≥1.0 mut/MB.

Conveniently the method of the present invention further comprising administering to a patient found to have a moderate response or strong response, an effective amount of the target agent.

According to the present invention there is provided a method for treating a patient suffering from proliferative disease, said method comprising carrying out a method according to the present invention using a tumour sample from said patient, developing a customised recommendation for treatment or continued treatment, based upon the overall score, and administering a suitable target agent, therapy or treatment to said patient.

According to the present invention there is provided a computer or machine-readable cassette programmed to implement the method according to the present invention.

According to the present invention there is provided a system for identifying patients suffering from proliferative disease who would respond to treatment using an agent targeting a cell pathway or components thereof comprising an immune-checkpoint comprising components of the PD1/PD-L1 pathway, an agent targeting DDR signalling pathway comprising PARP inhibitors, DDR inhibitors and cell cycle checkpoint inhibitors, or a combination of thereof, said system comprising:
a processor; and
a memory that stores code of an algorithm that, when executed by the processor, causes the computer system to:
receive data regarding tumour type of a sample;
receive data regarding level of expression of PD-L1 in the sample;
receive data regarding level of the tumour mutational burden in said sample;
receive data regarding level of DNA damage and repair related genes analysis based on the tumour type and PD-L1 levels;
analyse and transform the input levels via an algorithm to provide an output indicative of the level of susceptibility of said patient to treatment using the target agent; display the output on a graphical interface of the processor.

Conveniently instead of merely receiving the data, the memory further comprises code which allows at least one of the levels to be determined by the system.

Conveniently the memory further comprises code to provide a customised recommendation for the treatment of the patient, based upon the output.

Conveniently the customised recommendation is displayed on a graphical interface of the processor.

According to the present invention there is provided a non-transitory computer-readable medium storing instructions that, when executed by a processor, cause a computer system to identify patients suffering from proliferative disease who would respond to treatment using an agent targeting a cell pathway or components thereof comprising an immune-checkpoint comprising components of the PD1/PD-L1 pathway, an agent targeting DDR signalling pathway comprising PARP inhibitors, DDR inhibitors and cell cycle checkpoint inhibitors, or a combination of thereof, by:
receiving data regarding tumour type of a sample;
receiving data regarding level of expression of PD-L1 in the sample;
receiving data regarding level of the tumour mutational burden in said sample;
receiving data regarding level of DNA damage and repair related genes analysis based on the tumour type and PD-L1 levels;
analysing and transforming the input levels via an algorithm to provide an output indicative of the level of susceptibility of said patient to treatment using the target agent;
displaying the output on a graphical interface of the processor.

Conveniently the non-transitory computer-readable medium further comprises instructions which allows at least one of the levels to be determined by the system.

Conveniently the non-transitory computer-readable medium further stores instructions for developing a customised recommendation for treatment of the patient based upon the output and displaying the customised recommendation on a graphical interface of the processor.

Conveniently, the algorithm used in the present invention is shown diagrammatically in FIG. 1 and is as follows:

Scores are assigned to each of the analysed parameters:
i) a score of ‘0’ is applied in the absence of PD-L1 expression;
ii) a score of ‘1’ is applied in the presence <7 nLRPM but not 0 in relation to PD-L1 expression;
iii) a score of ‘2’ is applied in the presence 7-10 nLRPM in relation to PD-L1 expression;
iv) a score of ‘3’ is applied in the presence >10 nLRPM in relation to PD-L1 expression;
v) a score of ‘0’ is applied if the tumour mutational burden is ‘low’;
vi) a score of ‘1’ is applied if the tumour mutational burden is ‘high’;
vii) a score of ‘0’ is applied if there are no aberrations in the DNA damage and repair related genes analysis;
viii) a score of ‘1’ is applied if there is 1 aberration in the DNA damage and repair related genes analysis;
xi) a score of ‘2’ is applied if there are 2 aberrations in the DNA damage and repair related genes analysis;
x) a score of ‘3’ is applied if there are aberrations in the DNA damage and repair related genes analysis;
wherein an overall score of 0 is indicative of no susceptibility to the target agent, an overall score of 1-2 indicates a weak response, an overall score of 3-4 indicates a moderate response, and an overall score of 5 to 7 indicates a strong response.

Automation of the system minimises human error when calculating the output.

TABLE A
DDR signatures in relation to tumour type and PD-L1 positive cut-offs

| | DDR Signature ≥7 nLRPM | DDR Signature <7 nLRPM |
|---|---|---|
| Tissue | (PD-L1 IHC ≥10%) | (PD-L1 IHC <10%) |
| Bladder | TP53 | AKT2 ARID1A BRCA2 CDK12 |
| | | CREBBP MSH6 NBN PALB2 RB1 |
| | | SLX4 TP53 |
| Breast | AKT1 AKT2 ATM ATR BRCA1 | AKT1 AKT2 AKT3 ARID1A ATM |
| | TP53 | ATR AXL BAP1 BRCA1 BRCA2 |
| | | CHEK1 CHEK2 CREBBP FANCA |
| | | MLH1 NBN NF1 NOTCH1 |
| | | NOTCH2 PALB2 PMS2 PTEN |
| | | RAD50 RAD51D RB1 SETD2 |
| | | TP53 |
| Cervical | DDR2 TP53 | ARID1A BAP1 BRCA2 NBN |
| | | NOTCH3 PTEN RAD51B |
| Colorectal | ATM ATR CREBBP IDH2 PTEN | AKT1 AKT2 ALK ARID1A ATM |
| | RNF43 TP53 | ATR ATRX BRCA1 BRCA2 CDK12 |
| | | CHEK2 FANCA FANCD2 MLH1 |
| Bladder | TP53 | AKT2 ARID1A BRCA2 CDK12 |
| | | CREBBP MSH6 NBN PALB2 RB1 |
| | | SLX4 TP53 |
| | | MRE11 NBN NF1 PMS2 POLE |
| | | PTEN RAD51C RAD51D RB1 |
| | | SETD2 TP53 |
| CUP | ATM BRCA1 TP53 | AKT3 ARID1A BAP1 BRCA2 |
| | | FANCI IDH1 MLH1 PTEN SETD2 |
| | | TP53 |
| Endometrial | AKT2 TP53 | AKT1 ALK ARID1A ATM ATR |
| | | ATRX BRCA2 CREBBP MSH2 |
| | | MSH6 NF1 POLE PTEN RAD51C |
| | | TP53 |
| Gallbladder | — | ARID1A FANCD2 TP53 |
| Gastric | AKT1 ARID1A ATM ATR AXL | ARID1A ATM ATR BAP1 PTEN |
| | TP53 | RAD50 TP53 |
| Glioblastoma/Glioma | ATM NBN NF1 PTEN RB1 | ARID1A ATM ATR ATRX BRCA2 |
| | SETD2 TP53 | CREBBP FGFR3 IDH1 MLH1 |
| | | MRE11 NBN NF1 PTEN RB1 |
| | | SETD2 TP53 |
| GOJ | — | AKT2 NBN NF2 POLE TP53 |
| Head and Neck | ATM BRCA2 TP53 | BAP1 FGFR3 NF1 NOTCH1 PTEN |
| | | SETD2 TP53 |
| Kidney | SLX4 | BAP1 PTEN SETD2 SMARCB1 |
| | | TP53 |
| Bladder | TP53 | AKT2 ARID1A BRCA2 CDK12 |
| | | CREBBP MSH6 NBN PALB2 RB1 |
| | | SLX4 TP53 |
| Liver | TP53 | ARID1A ATM BAP1 BRCA2 |
| | | CHEK2 FANCA NBN NF1 NF2 |
| | | PTEN RB1 TP53 |
| Lung | AKT2 ARID1A ATM ATR | AKT1 AKT2 ARID1A ATM ATR |
| | CHEK2 NBN NF1 NF2 | AXL BAP1 BRCA2 CHEK1 |
| | NOTCH1 PTEN RB1 TP53 | CREBBP DDR2 FANCA FANCD2 |
| | | MLH1 MRE11 NBN NF1 |
| | | NOTCH3 PALB2 RAD50 RB1 RET |
| | | SETD2 SMARCA4 TP53 |
| Melanoma | PTEN | ATM ATR BAP1 CHEK1 FANCD2 |
| | | FANCI MRE11 NF1 PTEN SETD2 |
| | | TP53 |
| Mesothelioma | BAP1 NF2 TP53 | ATM BAP1 NF2 TP53 |
| Oesophageal | ARID1A BRCA2 PTEN TP53 | ATM ATRX CREBBP PTEN SETD2 |
| | | TP53 |
| Other | ARID1A IDH1 NOTCH1 PMS2 | ARID1A BRCA2 NBN RAD51B |
| | SETD2 TP53 | SMARCB1 TP53 |
| Ovarian | ARID1A ATM BRCA1 BRCA2 | AKT2 ARID1A ATM ATR AXL |
| | FANCD2 MLH1 MSH6 NF1 | BRCA1 BRCA2 CDK12 FANCI |
| | NOTCH1 PTEN RB1 TP53 | NBN NF1 POLE PTEN TP53 |
| Pancreatic | AKT2 ATM BRCA2 NF2 TP53 | ARID1A ATM BRCA2 CDK12 |
| | | CHEK2 NBN NF2 PTEN RB1 |
| | | RNF43 TP53 |
| Bladder | TP53 | AKT2 ARID1A BRCA2 CDK12 |
| | | CREBBP MSH6 NBN PALB2 RB1 |
| | | SLX4 TP53 |
| Prostate | — | AKT1 ARID1A ATM ATR BAP1 |
| | | BRCA2 CDK12 CHEK2 FANCA |
| | | FANCD2 FGFR3 PALB2 PTEN |
| | | RAD50 RB1 TP53 |
| Sarcoma | NF1 TP53 | ALK ATM ATRX BRCA2 CREBBP |
| | | IDH1 MRE11 NF1 NOTCH3 |
| | | PALB2 RAD51C RB1 SLX4 |
| | | SMARCB1 TP53 |
| Small bowel | NBN TP53 | NBN TP53 |
| Thyroid | NF1 TP53 | ATM PTEN RET |

### DETAILED DESCRIPTION OF THE INVENTION

The invention will now be particularly described by way of example with reference to the accompanying diagrammatic drawings in which:

FIG. 1 shows a diagrammatic representation of the method of the present invention which integrates PD-L1 expression levels as determined by normalised log RPM (nLRPM) with DDR mutation signature (DDR) and tumour mutation burden (TMB) to generate a polygenic prediction score (PPS) which is predictive of response to PD-L1 immune checkpoint targeted agents/immunotherapies

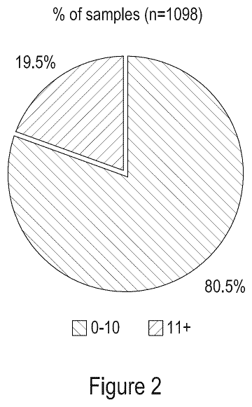

FIG. 2 shows a pie chart noting the frequency of samples with a PD-L1 tumour proportion score of 11+ compared to 0-10. Nineteen percent of tumours in the cohort of 1098 tumours analysed for PD-L1 expression by immunohistochemistry (IHC) show PD-L1 expression levels 1.0%.

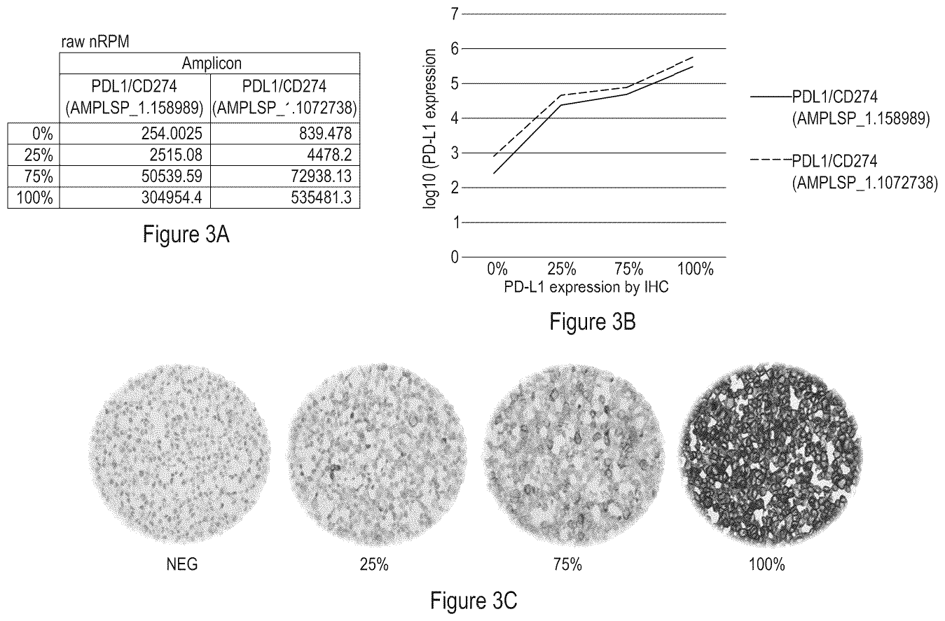

FIGS. 3A to C show a validation of analysis of PD-L1 mRNA expression by NGS (nLRPM) on stably expressing PD-L1 cell lines provided by Horizon Discovery Group plc. A CD274 (PD-L1) Reference Standard highly-characterized, biologically-relevant quality control material with negative (−), low positive (25%), intermediate positive (75%) and strong positive (100%) controlled protein expressing cell lines which can be utilised to test analytical (technical) performance of PD-L1 assays. The nLRPM readout shows strong correlation with PD-L1 expression as measured by IHC.

A) shows nLRPM counts from the two different amplicons targeting the PD-L1 gene.

B) shows PD-L1 nLRPM counts (mRNA) generated by the method of the present invention compared to PD-L1 protein expression assessed by IHC.

C) shows photomicrographs of four cell line controls immunohistochemically stained with an antibody against PD-L1 and expressing different levels of PD-L1 protein together with the observed tumour proportion score (TPS).

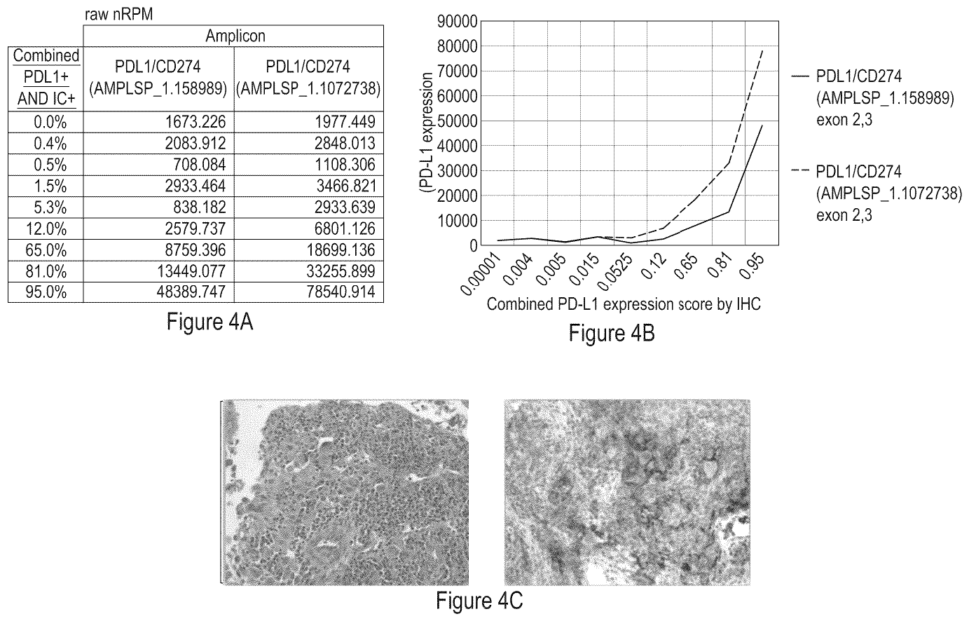

FIGS. 4A to C show a validation of analysis of PD-L1 mRNA expression by NGS (non-normalised RPM) with PD-L1 expression as assessed by IHC. Analysis was performed on 9 cases of non-small cell lung cancer (NSCLC). PD-L1 expression by IHC was determined by combining the PD-L1 tumour proportion score with the area of the section occupied by PD-L1 positive immune cells (ICs) [combined PD-L1 IHC score] using the algorithm [Combined PD-L1 expression score=tumour content×PD-L1 positive tumour cells+PIC score×PD-L1 positive ICs].

A) shows nRPM counts from the two different amplicons targeting the PD-L1 gene.

B) shows PD-L1 RPM counts (mRNA) generated by the method of the present invention compared to PD-L1 protein expression assessed by IHC.

C) shows photomicrographs of a representative sample of NSCLC stained with hematoxylin and eosin and immunohistochemically stained with an antibody against PD-L1.

The PD-L1 RPM expression levels show strong correlation with combined PD-L1 IHC expression levels.

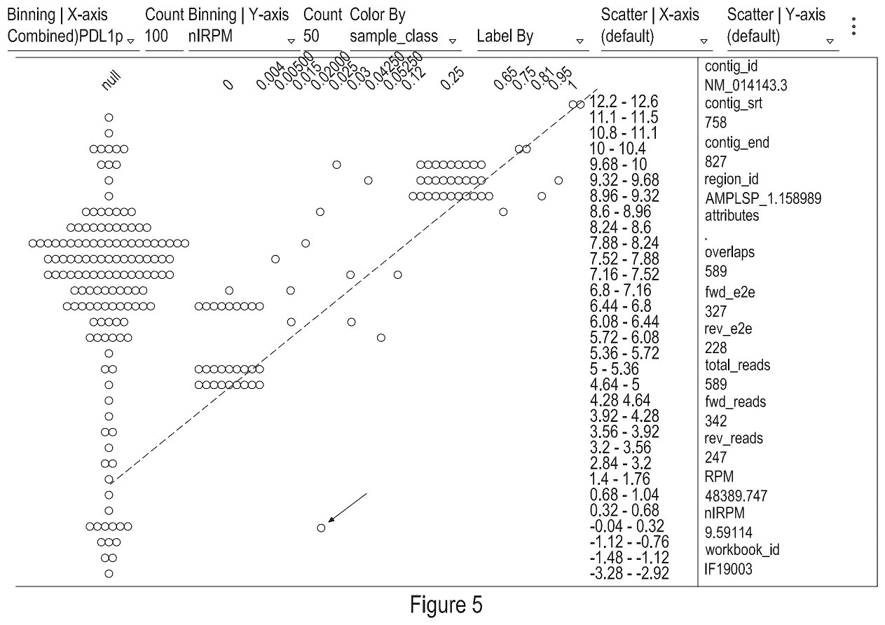

FIG. 5 shows log of normalised reads per million (nLRPM) and PD-L1 IHC expression (combined PD-L1 score as described above) The cohort tested includes PD-L1 Horizon control cell lines and 16 cases of non-small cell lung cancer (NSCLC). There is a strong correlation between PD-L1 nLRPM scores and PD-L1 IHC scores.

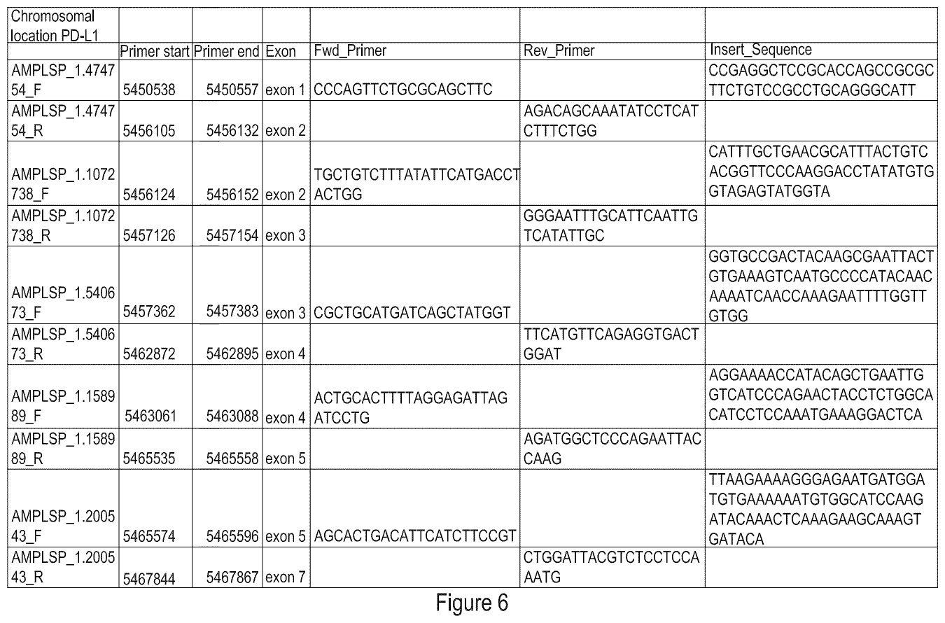

FIG. 6 sets out the primer sets which were designed to span the exon/intron boundaries across the PD-L1 gene. A person skilled in the art would be able to design their own primers based on the information given in the experimental protocol herein. However, it was found that AMPLSP_1.158989 and AMPLSP_1.1072738 provided a strong signal with notably a linear strong correlation with PD-L1 expression levels as measure by IHC.

In the present application, validation testing was performed on formalin fixed paraffin wax embedded tissue samples (PWET). Quantative analysis of RNA was performed in parallel and integrated with DNA DDR mutation analysis which has to date been a technical challenge because formalin fixation results in degradation of nucleic acid resulting in low DNA/RNA yields with low integrity and quality. The combined PD-L1-DDR NGS assay design is unique in being able to analyse PWET tissues and circumvent the problem of degraded DNA/RNA thereby enabling a combined PD-L1 mRNA gene expression and DDR signature to be generated.

Patient Demographics:

PD-L1 IHC expression analysis and genomic analysis of DDR genes was performed on a total of 1112 solid tumours. Details of the tumour cohort are shown in Table 1.

TABLE 1
Cancer type and histological classification of the study cohort.

| | Primary/Metastatic | |
|---|---|---|
| Cancer Type | lesion tested | N = 1112 |
| Breast | Primary carcinoma | 176 |
| | Invasive ductal (70) | |
| | Invasive lobular (5) | |
| | Metastatic carcinoma (101) | |
| Colorectal | Primary carcinoma | 177 |
| | Colorectal | |
| | adenocarcinoma (109) | |
| | Appendiceal | |
| | adenocarcinoma (4) | |
| | Appendiceal | |
| | neuroendocrine | |
| | carcinoma (1) | |
| | Anal squamous cell | |
| | carcinoma (5) | |
| | Metastatic carcinoma | |
| | Colorectal | |
| | adenocarcinoma (54) | |
| | Anal squamous cell | |
| | carcinoma (1) | |
| | Rectal squamous cell | |
| | carcinoma (1) | |
| | Appendiceal | |
| | adenocarcinoma (1) | |
| | Appendiceal | |
| | neuroendocrine | |
| | carcinoma (1) | |
| Ovarian | Primary carcinoma | 85 |
| | Serous (38) | |
| | Mucinous (2) | |
| | Endometrioid (2) | |
| | Clear cell (3) | |
| | Undifferentiated (2) | |
| | Malignant sex cord | |
| | stromal tumour (1) | |
| | Granulosa cell tumour | |
| | (1) | |
| | Metastatic carcinoma (36) | |
| Glioma | Astrocytoma | 81 |
| | Oligodendroglioma | |
| | Glioblastoma | |
| Lung | Primary carcinoma | 75 |
| | NSCLC (58) | |
| | SCLC (14) | |
| | Mucoepidermoid (1) | |
| | Metastatic carcinoma (2) | |
| Upper GI | Primary carcinoma | 75 |
| | Oesophageal | |
| | adenocarcinoma (23) | |
| | Oesophageal | |
| | squamous cell | |
| | carcinoma (10) | |
| | Oesophageal | |
| | lymphoepithelial | |
| | carcinoma (1) | |
| | Gastric | |
| | adenocarcinoma (25) | |
| | Gastric | |
| | neuroendocrine | |
| | carcinoma (1) | |
| | Gastro-oesophageal | |
| | junction | |
| | adenocarcinoma (6) | |
| | Metastatic carcinoma | |
| | Oesophageal (4) | |
| | Gastric (4) | |
| | GOJ (1) | |
| Pancreatic | Primary carcinoma | 71 |
| | Adenocarcinoma (41) | |
| | Anaplastic carcinoma | |
| | (1) | |
| | Adenosquamous | |
| | carcinoma (1) | |
| | Neuroendocrine | |
| | carcinoma (1) | |
| | Metastatic carcinoma (27) | |
| Sarcoma | Primary | 58 |
| | Leiomyosarcoma (11) | |
| | Liposarcoma (5) | |
| | Chordoma (3) | |
| | Ewing's sarcoma (3) | |
| | Pleomorphic sarcoma | |
| | (3) | |
| | Rhabdomyosarcoma | |
| | (3) | |
| | Angiosarcoma (2) | |
| | Chondrosarcoma (2) | |
| | Malignant peripheral | |
| | nerve sheath tumour | |
| | (2) | |
| | Other (11) | |
| | Metastatic sarcoma (12) | |
| Prostate | Primary carcinoma | 45 |
| | Adenocarcinoma (44) | |
| | Metastatic carcinoma (1) | |
| CUP | Metastatic carcinoma | 38 |
| | Poorly differentiated | |
| | carcinoma (9) | |
| | Adenocarcinoma (22) | |
| | Squamous cell | |
| | carcinoma (3) | |
| | Neuroendocrine | |
| | carcinoma (4) | |
| Head & Neck | Primary carcinoma | 34 |
| | Squamous cell | |
| | carcinoma (23) | |
| | Adenoid cystic | |
| | carcinoma (3) | |
| | Acinic cell carcinoma | |
| | (1) | |
| | Mucoepidermoid (3) | |
| | Salivary duct | |
| | carcinoma (3) | |
| | Low grade parotid | |
| | tumour (1) | |
| Liver | Primary carcinoma | 32 |
| | Cholangiocarcinoma | |
| | (19) | |
| | Biliary tract | |
| | adenocarcinoma (3) | |
| | Hepatocellular | |
| | carcinoma (7) | |
| | Hepatoblastoma (1) | |
| | Metastatic carcinoma | |
| | Hepatocellular | |
| | carcinoma (2) | |
| Bladder | Primary carcinoma | 24 |
| | Transitional cell | |
| | carcinoma (17) | |
| | Adenocarcinoma (4) | |
| | Urethral | |
| | adenocarcinoma (1) | |
| | Metastatic carcinoma | |
| | Transitional cell | |
| | carcinoma (2) | |
| Other | Primary tumours | 19 |
| | Vulva squamous cell | |
| | carcinoma (3) | |
| | Right buttock | |
| | squamous cell | |
| | carcinoma (1) | |
| | Mediastinal tumour | |
| | (1) | |
| | NUT midline | |
| | carcinoma (1) | |
| | Pecoma (1) | |
| | Merkel cell carcinoma | |
| | (1) | |
| | Neurocytoma (1) | |
| | Pseudomyxoma | |
| | peritonei (2) | |
| | Adrenal carcinoma (1) | |
| | Peritoneal high grade | |
| | serous carcinoma (1) | |
| | Testicular | |
| | adenocarcinoma/germ | |
| | cell tumour (1) | |
| | Yolk sac tumour (1) | |
| | Diffuse B-cell | |
| | lymphoma (1) | |
| | Teratoma (1) | |
| | Neurocytoma (1) | |
| | Choroid plexus | |
| | carcinoma (1) | |
| Endometrial | Primary carcinoma | 23 |
| | Adenocarcinoma (8) | |
| | Serous carcinoma (4) | |
| | Carcinosarcoma (4) | |
| | Metastatic carcinoma | |
| | Adenocarcinoma (7) | |
| Cervix | Primary carcinoma | 22 |
| | Squamous cell | |
| | carcinoma (12) | |
| | Adenocarcinoma (6) | |
| | Adenosquamous | |
| | carcinoma (1) | |
| | Metastatic carcinoma (3) | |
| Mesothelioma | Primary | 19 |
| | Epitheliod (17) | |
| | Sarcomatoid (2) | |
| | Biphasic (2) | |
| Kidney | Primary carcinoma | 18 |
| | Transitional cell | |
| | carcinoma (3) | |
| | Renal cell carcinoma | |
| | (11) | |
| | Metastatic carcinoma (4) | |
| Melanoma | Primary | 17 |
| | Malignant melanoma | |
| | (4) | |
| | Ocular spindle cell | |
| | malignant melanoma | |
| | (1) | |
| | Metastatic malignant | |
| | melanoma (12) | |
| Thyroid | Primary carcinoma | 9 |
| | Papillary (2) | |
| | Follicular (3) | |
| | Anaplastic (3) | |
| | Metastatic carcinoma (1) | |
| Small Bowel | Primary carcinoma | 7 |
| | Adenocarcinoma (6) | |
| | Metastatic carcinoma (1) | |
| Gallbladder | Primary carcinoma | 7 |
| | Adenocarcinoma (7) | |

TABLE 2
List of DDR genes analysed

| | |
|---|---|
| AKT1 | ALK |
| AKT2 | ARMT1 |
| AKT3 | ATAD5 |
| ARID1A | ATG7 |
| ATM | ATIC |
| ATR | AXL |
| ATRX | BIRC6 |
| BAP1 | BRD3 |
| BRCA1 | BRD4 |
| BRCA2 | CAPRIN1 |
| CDK12 | CCAR2 |
| CHEK1 | CCDC6 |
| CHEK2 | CDK5RAP2 |
| CREBBP | CHD9 |
| ERC1 | CIT |
| ERCC2 | CTNNB1 |
| FANCA | CUL1 |
| FANCD2 | DDR2 |
| FANCI | EBF1 |
| IDH1 | EIF3E |
| IDH2 | GNAS |
| MDM2 | HIP1 |
| MDM4 | HMGA2 |
| MLH1 | IRF2BP2 |
| MRE11A | MED12 |
| MSH2 | NF1 |
| MSH6 | NF2 |
| NBN | NOTCH1 |
| NSD1 | NOTCH2 |
| PALB2 | NOTCH3 |
| PMS2 | NOTCH4 |
| POLE | NPM1 |
| POLH | OFD1 |
| PPM1G | RNF43 |
| PTEN | SLX4 |
| RAD18 | SPOP |
| RAD50 | TACC1 |
| RAD51 | TACC3 |
| RAD51B | TERF2 |
| RAD51C | TMEM106B |
| RAD51D | UBE2L3 |
| RB1 | USP10 |
| SETD2 | WDR48 |
| SMARCA4 | XPO1 |
| SMARCB1 | YAP1 |
| TERT | ZEB2 |
| TP53 | ZMYND8 |
| TP53BP1 | |

TABLE 3
Fusions

| | Fusion | |
|---|---|---|
| | and | |
| Gene | partner | Sequence |
| AKT2 | BCAM- | CTCCTGCTCCTCGTCGTTGCTGTCTTCTACTGCGTGAGACGCAAA |
| | AKT2.B13A5 | GGGGGCCCCTGCTGCCGCCAGCGGCGGGAGAAGGGGGCTCCG |
| | | GAGGAGTGGATGCGGGCCATCCAGATGGTCGCCAACAGCCT |
| | ZNF226- | GACGACGTAGCAGCCATCTTTTCCCTGGCTTTGGTGATTCAGCCC |
| | AKT2.Z2A5 | TGACTTCTCAAAAAGCACTGCACAGAGGAGGAGGCAGCAGAAC |
| | | CCCATGGAGGAGTGGATGCGGGCCATCCAGATGGTCGCCAACA |
| | | GCCT |
| BRCA1 | BRCA1- | AGTCTGGGCCACACGATTTGACGGAAACATCTTACTTGCCAAGG |
| | BRCA1. | CAAGATCTAGATGCTCGTGTACAAGTTTGCCAGAAAACACCACAT |
| | B15B17. | CACTTTAACTAATCTAATTACTGAAGAGACTACTCATGTTGTTATG |
| | V16 | AAAACAGATGCTGAG |
| | BRCA1- | GGGTGACCCAGTCTATTAAAGAAAGAAAAATGCTGAATGAGGG |
| | BRCA1.B | TGTCCACCCAATTGTGGTTGTGCAGCCAGATGCCTGGACAGAGG |
| | 19B23.V20 | ACAATGGCTTCCATG |
| | BRCA1- | CCTGGAAGTAATTGTAAGCATCCTGAAATAAAAAAGCAAGAATA |
| | BRCA1. | TGAAGAAGTAGTTCAGACTGTTAATACAGATTTCTCTCCATATCT |
| | B10B14. | GATTTCAGATAACTTAGAACAGCCTATGGGAATATTAACTTCACA |
| | V11 | GAAAAGTAGTGAATACCCTATAAGCCAGAATCCA |
| | BRCA1- | CCTTCACAGTGTCCTTTATGTAAGAATGATATAACCAAAAGGTCA |
| | BRCA1.B | TCCCCTTCTAAATGCCCATCATTAGATGATAGGTGGTACATGCAC |
| | 4B15.V5 | AGTTGCTCTGGGAGTCTTCAGAATAGA |
| | es | |
| | BRCA1- | GAACTGTGAGAACTCTGAGGACAAAGCAGCGGATACAACCTCAA |
| | BRCA1. | AAGACGTCTGTCTACATTGAATTGGCAGAGGGATACCATGCAAC |
| | B7B12. | ATAACCTGATAAAGCTCCAGCAGGAAATGGCTGAACTAGAAGCT |
| | V8es | GTGT |
| | BRCA1- | GAACTGTGAGAACTCTGAGGACAAAGCAGCGGATACAACCTCAA |
| | BRCA1. | AAGACGTCTGTCTACATTGAATTGGTATTAACTTCACAGAAAAGT |
| | B7B14. | AGTGAATACCCTATAAGCCAGAATCCA |
| | V8es | |
| | BRCA1- | AGTCTGGGCCACACGATTTGACGGAAACATCTTACTTGCCAAGG |
| | BRCA1. | CAAGATCTAGATGCTGAGTTTGTGTGTGAACGGACACTGAAATA |
| | B15B18 | TTTTCTAGGAATTGCGGGAGGAAAATG |
| | BRCA1- | GAAGTCAGAGGAGATGTGGTCAATGGAAGAAACCACCAAGGTC |
| | BRCA1. | CAAAGCGAGCAAGAGAATCCCAGGACAGAAAGGGTGTCCACCC |
| | B20B23. | AATTGTGGTTGTGCAGCCAGATGCCTGGACAGAGGACAATGGCT |
| | V21es | TCCATG |
| BRCA2 | BRCA2- | TGCATCATGTTTCTTTAGAGCCGATTACCTGTGTACCCTTTCGGGC |
| | BRCA2. | TCTGTGTGACACTCCAGGTGTGGATCCAAAGCTTATTTCTAGAAT |
| | B13B17. | TTGGGTTTATAATCACTATAGATGGATCATATGGAAACTGGCAGC |
| | V14 | TATGGAATGTGCC |
| | BRCA2- | GTCAGCTTACTCCGGCCAAAAAAGAACTGCACCTCTGGAGCGGA |
| | | TTTAGGACCAATAAGTCTTAATTGGTTTGAAGAACTTTCTTCAGA |
| | BRCA2. | AGCTCCACCCTATAATTCTGAACCTGCAGAAGAATCTGAACATAA |
| | B1B3.V2 | A |
| | BRCA2- | CCATCACGTGCACTAACAAGACAGCAAGTTCGTGCTTTGCAAGAT |
| | BRCA2. | GGTGCAGAGCTTTATGAAGCAGTGAAGAATGCAGCAGACCCAG |
| | B21B25. | CTTACCTTGAGGACTTGCCCCTTTCGTCTATTTGTCAGACGAATGT |
| | V22 | TACAATTTACTGGCA |
| | BRCA2- | TTCTGAAAGTCTAGGAGCTGAGGTGGATCCTGATATGTCTTGGTC |
| | BRCA2. | AAGTTCTTTAGCTACACCACCCACCCTTAGTTCTACTGTGCTCATA |
| | B7B10.V8 | GGATTTGGAAAAACATCAGGGAATTCATTTAAAGTAAATAGCTG |
| | | CAAAGACCACATTGG |
| | BRCA2- | ACGAGGCATTGGATGATTCAGAGGATATTCTTCATAACTCTCTAG |
| | BRCA2. | ATAATGATGAATGTAGCACGCATTCACATTCCTTACACAAAGTTA |
| | B11B11.D | AGGGAGTGTTAGAGGAATTTGATTTAATCAGAACTGAGCATAGT |
| | | CTTCACTATTCACCTACGTCTAGACAA |
| | BRCA2- | GCATGTCTAACAGCTATTCCTACCATTCTGATGAGGTATATAATG |
| | BRCA2. | ATTCAGGATATGGTTTATCAAGGGATGTCACAACCGTGTGGAAG |
| | B11B22.V12 | TTGCGTATTGTAAGCTATTCA |
| | BRCA2- | ACTTGATTCTGGTATTGAGCCAGTATTGAAGAATGTTGAAGATCA |
| | BRCA2. | AAGTCCTTTATCACTTTGTATGGCCAAAAGGAAGTCTGTTTCCAC |
| | B11B27.V12 | ACCTGTCTCAGCCCAGATGACTTCAAAGTCTTGTAAAGGGGAG |
| ERC1 | ERC1- | CCAGCTTCCTATAACTTGGACGATGACCAGGCGGCTTGGGAGAA |
| | RET.E17R12 | TGAGCTGCAGAAGATGACCCGGGGGCAGGAGGATCCAAAGTGG |
| | | GAATTCCCTCGGAAGAACTTGGTTCTTGGAAAAACTCTAGGAGA |
| | | AGGCGAATTTGG |
| | ERC1- | GGCTTAAGACACTAGAGATTGCTTTGGAGCAGAAGAAGGAGGA |
| | RET.E11R12 | GTGTCTGAAAATGGAATCACAATTGAAAAAGGAGGATCCAAAGT |
| | | GGGAATTCCCTCGGAAGAACTTGGTTCTTGGAAAAACTCTAGGA |
| | | GAAGGCGAATTTGG |
| | ERC1- | CAGGCAGAAGTTGATCGACTCTTAGAAATCTTGAAGGAGGTGGA |
| | ROS1. | AAATGAGAAGAATGACAAAGATAAGAAGATAGCTGAGTTGGAA |
| | E11R36 | AGTACTCTTCCAACCCAAGAGGAGATTGAAAATCTTCCTGCCTTC |
| | | C |
| | ERC1- | GCAGTCTCTGGCAGAAAAGGAAACTCACTTGACTAATCTTCGGG |
| | PDGFRB. | CAGAGAGAAGGAAACACTTAGAGGAAGTTCTGGAGATGAAGTG |
| | E15P10 | TCCACGTGAGCTGCCGCCCACGCTGCTGGGGAACAGTTCCGAAG |
| | | AGGAGAGCCAGC |
| | ERC1- | GCAGTCTCTGGCAGAAAAGGAAACTCACTTGACTAATCTTCGGG |
| | PDGFRB. | CAGAGAGAAGGAAACACTTAGAGGAAGTTCTGGAGATGAACCT |
| | E15P11 | TGCCCTTTAAGGTGGTGGTGATCTCAGCCATCCTGGCCCTGGTG |
| | | GTGCTCACCATCATCTCCCTTATCATCCTCATCATGCTTTGGC |
| | ERC1- | AAAGAAGAGTGCACAAATGTTAGAGGAGGCGCGACGACGGGA |
| | BRAF. | GGACAATCTCAACGACAGCTCTCAGCAGCTACAGAAAGCCTTAC |
| | E12B10 | AGAAATCTCCAGGACCTCAGCGAGAAAGGAAGTCATCTTCATCC |
| | | TCAGAAGACAGGAATCGAATGAAAACACT |
| | ERC1- | CCAGCTTCCTATAACTTGGACGATGACCAGGCGGCTTGGGAGAA |
| | BRAF. | TGAGCTGCAGAAGATGACCCGGGGGCAGCCAGCAGATGAAGAT |
| | E17B8 | CATCGAAATCAATTTGGGCAACGAGACCGATCCTCATCAGCTCCC |
| | | AATGTGCA |
| | ERC1- | AAAGAAGAGTGCACAAATGTTAGAGGAGGCGCGACGACGGGA |
| | RET.E12R12 | GGACAATCTCAACGACAGCTCTCAGCAGCTACAGGAGGATCCAA |
| | | AGTGGGAATTCCCTCGGAAGAACTTGGTTCTTGGAAAAACTCTA |
| | | GGAGAAGGCGAATTTGG |
| | ERC1- | GCTGGAGAGATACATGACCTCAAGGACATGTTGGATGTGAAGG |
| | RET.E7R12 | AGCGGAAGGTTAATGTTCTTCAGAAGAAGGAGGATCCAAAGTG |
| | | GGAATTCCCTCGGAAGAACTTGGTTCTTGGAAAAACTCTAGGAG |
| | | AAGGCGAATTTGG |
| NSD1 | NSD1- | GGGTCAAAGATCCTTGCATCTAATAGTATCATCTGCCCTAATCAC |
| | NOTCH4. | TTTACCCCTAGGCGGGGCTGCCGAAATCATGAGCATGTTAATGTT |
| | N14N18 | AGCTGGTGCTTTGTGTGCTCAGAAGGCATAGACGTCTCTTCCCTT |
| | | TGCCACAATGGAGGC |
| POLH | ESR1- | GCTTACTGACCAACCTGGCAGACAGGGAGCTGGTTCACATGATC |
| | POLH.E6P2 | AACTGGGCGAAGAGGGTGCCAGAAAAATGGCTACTGGACAGGA |
| | | TCGAGTGGTTGCTCTCGTGGACATGGACTGTTTTTTTGTTCAAGT |
| | | GGAGCAGCG |
| PPM1G | PPM1G- | GCTTCTCCGCCATGCAAGGCTGGCGCGTCTCCATGGAGTGATGG |
| | ALK.P1A18 | AAGGCCACGGGGAAGTGAATATTAAGCATTATCTAAACTGCAGT |
| | | CACTGTGAGGTAG |
| PTEN | PTEN- | CTGCAGAAAGACTTGAAGGCGTATACAGGAACAATATTGATGAT |
| | BTAF1.P2B2 | GTAGTAAGGCTAGATCGCCTTTTTATTTTACTGGATACTGGCACT |
| | | ACTCCTGTTACAAGAAAAGCTGCTGCACAGCAAC |
| | PTEN- | CTGCAGAAAGACTTGAAGGCGTATACAGGAACAATATTGATGAT |
| | SHROOM4.P2S3 | GTAGTAAGGAGGAACGCCCCTGTCAGTAGGCCGCACTCATGGCA |
| | | TGTGGCCAAGCTGCTGGAGGGATGCCCTGAAGCAGCCACCACCA |
| | | TGCATTTCCCTTCTGAAG |
| | PTEN- | CTGCAGAAAGACTTGAAGGCGTATACAGGAACAATATTGATGAT |
| | SHROOM4.P3S4 | GTAGTAAGGTTTTTGGATTCAAAGCATAAAAACCATTACAAGATA |
| | | TACAATCTTGACGTGTGTGTGCAGTGGTGTCCACTCTCCCGGCAT |
| | | TGCAGCACCGAGAAAAGCAGCTCCATTGGCA |
| RAD18 | RAD18- | CAACAGCTCATTAAAAGGCACCAAGAATTTGTACACATGTACAAT |
| | BRAF.R7B10 | GCCCAATGCGATGCTTTGCATCCTAAATCAGGATCAACCACAGGT |
| | | TTGTCTGCTACCCCCCCTGCCTCATTACCTGGCTCACTAACTAACG |
| | | TG |
| RAD51 | CHD9- | GCTCGGAGTTGGCATTCATCATTTTCTAATCATCAGCATTTACATG |
| | RAD51B.C2R8 | ACAGAAATCACCTATGTTTACAGCGACAGGTTATCTTGACGAATC |
| | | AGATTACAACCCATCTGAGTGGAGCCCTGGCTTCTCAGGCAGAC |
| | | CTGGTGTCTCCAGCTG |
| | EIF3E- | CTCGCATCGCGCACTTTTTGGATCGGCATCTAGTCTTTCCGCTTCT |
| | RAD51B.E1R5 | TGAATTTCTCTCTGTAAAGGAGATTACAGGTCCACCAGGTTGTGG |
| | | AAAAACTCAGTTTTGTATAATGATGAGCATTTTGGCTACATTACC |
| | | CACCAACATGGGAG |
| | HMGA2- | CTAAAGCAGCTCAAAAGAAAGCAGAAGCCACTGGAGAAAAACG |
| | RAD51B.H3R11 | GCCAAGAGGCAGACCTAGGAAATGGAGACAACATTTTGCTCTGT |
| | | CACCCAAGCTGAACTGAACTGGGCTCCAGAAATCCTCCCACCTCA |
| | | GCCTCCTGAGCAGCTAGGACTACAGATGTGCCACCA |
| | NPC2- | GTTATCCGCGATGCGTTTCCTGGCAGCTACATTCCTGCTCCTGGC |
| | RAD51B.N1R9 | GCTCAGCACCGCTGCCCAGGCCGAACCGGTGCAGTTCAAGGACT |
| | | GCGGCACTTCTGGATCCAGCTGTGTGATAGCCGCACTAGGAAAT |
| | | ACCTGGAGTCACAGTGT |
| | PCNX- | CAGGCCACCTTCGTGAACGCGCTGCACCTCTACCTGTGGCTCTTT |
| | RAD51B.P1R8 | CTGCTGGGCCTGCCCTTCACCCTCTACATGGTTATCTTGACGAATC |
| | | AGATTACAACCCATCTGAGTGGAGCCCTGGCTTCTCAGGCAGAC |
| | | CTGGTGTCTCCAGCTG |
| RAD51B | CHD9- | GCTCGGAGTTGGCATTCATCATTTTCTAATCATCAGCATTTACATG |
| | RAD51B. | ACAGAAATCACCTATGTTTACAGCGACAGGTTATCTTGACGAATC |
| | C2R8 | AGATTACAACCCATCTGAGTGGAGCCCTGGCTTCTCAGGCAGAC |
| | | CTGGTGTCTCCAGCTG |
| | EIF3E- | CTCGCATCGCGCACTTTTTGGATCGGCATCTAGTCTTTCCGCTTCT |
| | RAD51B.E1R5 | TGAATTTCTCTCTGTAAAGGAGATTACAGGTCCACCAGGTTGTGG |
| | | AAAAACTCAGTTTTGTATAATGATGAGCATTTTGGCTACATTACC |
| | | CACCAACATGGGAG |
| | HMGA2- | CTAAAGCAGCTCAAAAGAAAGCAGAAGCCACTGGAGAAAAACG |
| | RAD51B.H3R11 | GCCAAGAGGCAGACCTAGGAAATGGAGACAACATTTTGCTCTGT |
| | | CACCCAAGCTGAACTGAACTGGGCTCCAGAAATCCTCCCACCTCA |
| | | GCCTCCTGAGCAGCTAGGACTACAGATGTGCCACCA |
| | NPC2- | GTTATCCGCGATGCGTTTCCTGGCAGCTACATTCCTGCTCCTGGC |
| | RAD51B.N1R9 | GCTCAGCACCGCTGCCCAGGCCGAACCGGTGCAGTTCAAGGACT |
| | | GCGGCACTTCTGGATCCAGCTGTGTGATAGCCGCACTAGGAAAT |
| | | ACCTGGAGTCACAGTGT |
| | PCNX- | CAGGCCACCTTCGTGAACGCGCTGCACCTCTACCTGTGGCTCTTT |
| | RAD51B.P1R8 | CTGCTGGGCCTGCCCTTCACCCTCTACATGGTTATCTTGACGAATC |
| | | AGATTACAACCCATCTGAGTGGAGCCCTGGCTTCTCAGGCAGAC |
| | | CTGGTGTCTCCAGCTG |
| RB1 | RB1- | CTGAGCACCCAGAATTAGAACATATCATCTGGACCCTTTTCCAGC |
| | RB1.R20R24 | ACACCCTGCAGAATGAGTATGAACTCATGAGAGACAGGCATTTG |
| | | GACCAAAATCTTAGTATCAATTGGTGAATCATTCGGGACTTCTGA |
| | | GAAGTTCCAGAAAATAAATCAGATGGTATGTAACAGCGACCGTG |
| | | TGCTCAAAAGAAGTGCTGAAG |
| | RB1- | TGTTCCATGTATGGCATATGCAAAGTGAAGAATATAGACCTTAAA |
| | RB1.R21R23 | TTCAAAATCATTGTAACAGCATACAAGGATCTTCCTCATGCTGTTC |
| | | AGGAGCCCCCTACCTTGTCACCAATACCTCACATTCCTCGAAGCC |
| | | CTTACAAGTTTC |
| | RB1- | TGTTCCATGTATGGCATATGCAAAGTGAAGAATATAGACCTTAAA |
| | RB1.R21R25 | TTCAAAATCATTGTAACAGCATACAAGGATCTTCCTCATGCTGTTC |
| | | AGGAGACTTCTGAGAAGTTCCAGAAAATAAATCAGATGGTATGT |
| | | AACAGCGACCGTGTGCTCAAAAGAAGTGCTGAAG |
| | RB1.E4E5.WT | AATGCTATGTCAAGACTGTTGAAGAAGTATGATGTATTGTTTGCA |
| | | CTCTTCAGCAAATTGGAAAGGACATGTGAACTTATATATTTGACA |
| | | CAACCCAGCAGTTCGATATCTACTGAAATAAATTCTGCATTGGTG |
| | | CTAAAAGTTTCTTG |
| | RB1. | AGGATCAGATGAAGCAGATGGAAGTAAACATCTCCCAGGAGAG |
| | E26E27.WT | TCCAAATTTCAGCAGAAACTGGCAGAAATGACTTCTACTCGAACA |
| | | CGAATGCAAAAGCAGAAAATGAATGATAGCATGGATACCTCAAA |
| | | CAAGGAAGAGAAATGA |
| | RB1.R21 | TGTTCCATGTATGGCATATGCAAAGTGAAGAATATAGACCTTAAA |
| | R22R23.WT | TTCAAAATCATTGTAACAGCATACAAGGATCTTCCTCATGCTGTTC |
| | | AGGAGACATTCAAACGTGTTTTGATCAAAGAAGAGGAGTATGAT |
| | | TCTATTATAGTATTCTATAACTCGGTCTTCATGCAGAGACTGAAA |
| | | ACAAATATTTTGCAGTATGCTTCCACCAGGCCCCCTACCTTGTCAC |
| | | CAATACCTCACATTCCTCGAAGCCCTTACAAGTTTC |
| TERT | CCDC127- | ATTCCAGGGCGGATGGTGGTGATGGAAGCAGGTGGAATTATGC |
| | TERT.C2T3 | CCTGTTGGTTCCAATGCTGGGATTGGCTGCTTTTCGGGTTGGCTG |
| | | TGTTCCGGCCGCAGAGCACCGTCTGCGTGAGGAGATCCTGGCCA |
| | | AGTTCCTGCACTGGCT |
| | GLIS3- | CTATAAACTGCTGATCCACATGAGAGTCCACTCTGGGGAGAAGC |
| | TERT.G3T3 | CCAACAAGTGTACGGGGTTGGCTGTGTTCCGGCCGCAGAGCACC |
| | | GTCTGCGTGAGGAGATCCTGGCCAAGTTCCTGCACTGGCT |
| | MTMR12- | CAAAGGCAACATGAAGTACAAAGCAGTGAGTGTCAACGAAGGC |
| | TERT.M7T3 | TATAAAGTCTGTGAGAGGGGTTGGCTGTGTTCCGGCCGCAGAGC |
| | | ACCGTCTGCGTGAGGAGATCCTGGCCAAGTTCCTGCACTGGCT |
| | TRIO- | GCAGCAGCCAGCCTGATACGATTTCCATCGCCTCACGGACGTCTC |
| | TERT.T33T2 | AGAACACGCTGGACAGCGATAAGGTGTCCTGCCTGAAGGAGCT |
| | | GGTGGCCCGAGTGCTGCAGAGGCTGTGCGAGCGCGGCGCGAAG |
| | | AACGTGCTGGCCTTC |
| | SLC12A7- | CATGCCCACCAACTTCACCGTGGTGCCCGTGGAGGCTCACGCCG |
| | TERT.S1T3 | ACGGCGGCGGGGACGAGACTGCCGAGCGGACGGAGGCTCCGG |
| | | GCACCCCCGAGGGCCCCGAGCCCGAGCGCCCCAGCCCGGGGGT |
| | | TGGCTGTGTTCCGGCCGCAGAGCACCGTCTGCGTGAGGAGATCC |
| | | TGGCCAAGTTCCTGCACTGGCT |
| | TTLL7- | CGGGCTGGGCTTTCCTCACCCGGGGGTTGGCTGTGTTCCGGCCG |
| | TERT.T1T3 | CAGAGCACCGTCTGCGTGAGGAGATCCTGGCCAAGTTCCTGCAC |
| | | TGGCT |
| | TERT- | ACGGCCTATTCCCCTGGTGCGGCCTGCTGCTGGATACCCGGACCC |
| | ALK.T11A5 | TGGAGGTGCAGAGCGACTACTCCAGTTGGACAGTGCTCCAGGG |
| | | AAGAATCGGGCGTCCAGACAACCCATTTCGAGTGGCCCTGGA |
| | ADAMTS16- | AGTAAATATCGCAGCTGCACGATTAATGAAGATACAGGTCTTGG |
| | TERT.A8T3 | ACTGGCCTTCACCATTGCCCATGAGTCTGGACACAAGGGTTGGCT |
| | | GTGTTCCGGCCGCAGAGCACCGTCTGCGTGAGGAGATCCTGGCC |
| | | AAGTTCCTGCACTGGCT |
| TP53 | TP53- | GAGCTGAATGAGGCCTTGGAACTCAAGGATGCCCAGGCTGGGA |
| | NTRK1. | AGGAGCCAGGGGGGAGCAGGGCTCACTCCAGTCCCGGCCAGTG |
| | T10N9 | TGCAGCTGCACACGGCGGTGGAGATGCACCACTGGTG |
| | TP53- | CAGAGGAAGAGAATCTCCGCAAGAAAGGGGAGCCTCACCACGA |
| | NTRK1. | GCTGCCCCCAGGGAGCACTAAGCGAGTCCCGGCCAGTGTGCAGC |
| | T8N9 | TGCACACGGCGGTGGAGATGCACCACTGGTG |
| | TP53- | CTCCTCTCCCCAGCCAAAGAAGAAACCACTGGATGGAGAATATTT |
| | NTRK1. | CACCCTTCAGTCCCGGCCAGTGTGCAGCTGCACACGGCGGTGGA |
| | T9N9 | GATGCACCACTGGTG |
| | TP53- | TCCCCTCCTTCTCCCTTTTTATATCCCATTTTTATATCGATCTCTTAT |
| | NTRK1. | TTTACAATAAAACTTTGCTGCCACCTGTGTGTCTGAGGGGTGTCC |
| | T11N9 | CGGCCAGTGTGCAGCTGCACACGGCGGTGGAGATGCACCACTG |
| | | GTG |
| TP53BP1 | TP53BP1- | CAAGCGAGGTCGCAAGTCTGCCACAGTAAAACCTGCCTTGCCCTT |
| | PDGFRB. | TAAGGTGGTGGTGATCTCAGCCATCCTGGCCCTGGTGGTGCTCA |
| | T23P11 | CCATCATCTCCCTTATCATCCTCATCATGCTTTGGC |
| ALK | EML4-ALK. | GTGCTGTCTCAATTGCAGGAAAAGAAACTCTTTCATCTGCTGCTA |
| | E3p53insA20 | AAAGTGCTTCAAGGGCCAGGCTGCCAGGCCATGTTGCAGCTGAC |
| | | CACCCACCTGCAGTGTACCGCCGGAAGCACCAGGAGCTGCAAGC |
| | | CATGCAGATGGAGCTGCAGAGCCCTGAG |
| | PPM1G- | GCTTCTCCGCCATGCAAGGCTGGCGCGTCTCCATGGAGTGATGG |
| | ALK.P1A18 | AAGGCCACGGGGAAGTGAATATTAAGCATTATCTAAACTGCAGT |
| | | CACTGTGAGGTAG |
| | KANK2- | CCAGGAGGTGGTGGAGACAATGTGCCCAGTGCCCGCTGCAGCT |
| | ALK.K4A16 | ACCAGCAACGTCCATATGGTGAAGAAGATTAGCATCACAGAGCG |
| | | AAGCTGCGATGGAGCAGCAGGTGGTGGAGGTGGCTGGAATGAT |
| | | AACACTTCCTTGCTCTGGG |
| | KIF5B- | ATCGCAAACGCTATCAGCAAGAAGTAGATCGCATAAAGGAAGCA |
| | ALK.K24A19 | GTCAGGTCAAAGAATATGGCCAGAAGAGGGCATTCTGCACAGAT |
| | | TGTGTCACCCACCCCGGAGCCACACCTGCCACTCTCGCTGATCCT |
| | | CTCTGTGGTGACCT |
| | MCFD2- | GGACCAGCTCCGGCATGCGGTCCCAGTGGCCCTCGGCGCGGCA |
| | ALK.M1A20 | GCGCTCCAGCTCGCTCTCCACCTTCAGTGTACCGCCGGAAGCACC |
| | | AGGAGCTGCAAGCCATGCAGATGGAGCTGCAGAGCCCTGAG |
| | STK32B- | CCCGCTGAATGGACACCTGCAGCACTGTTTGGAGACTGTCCGGG |
| | ALK.S11A20 | AGGAATTCATCATATTCAACAGAGAGAATGTACCGCCGGAAGCA |
| | | CCAGGAGCTGCAAGCCATGCAGATGGAGCTGCAGAGCCCTGAG |
| | CAD- | GCTTCCTGATGGCCGCTTCCATCTGCCGCCCCGAATCCATCGAGC |
| | ALK.C35A20 | CTCCGACCCAGGTTTGCCAGTGTACCGCCGGAAGCACCAGGAGC |
| | | TGCAAGCCATGCAGATGGAGCTGCAGAGCCCTGAG |
| | GFPT1- | CTCAGCGTGATCCCTTTACAGTTGCTGGCTTTCCACCTTGCTGTGC |
| | ALK.G18A20.1 | TGAGAGGCTATGATGACCTCCTCCATCAGTGACCTGAAGGAGGT |
| | | GCCGCGGAAAAACATCACCCTCATTCGGGGTCTGGGCCATGGCG |
| | | CCTTTGGGGAGGTGTATGAAGGCCAGGTGTCCGGAATGCCC |
| | TPR- | GGAAGAATTAGAAGCTGAGAAAAGAGACTTAATTAGAACCAAT |
| | ALK.T4A20 | GAGAGACTATCTCAAGAACTTGAATACTTAACAGTGTACCGCCG |
| | | GAAGCACCAGGAGCTGCAAGCCATGCAGATGGAGCTGCAGAGC |
| | | CCTGAG |
| | EML4- | CCTTCCTGGCTGTAGGATCTCATGACAACTTTATTTACCTCTATGT |
| | ALK.E19A20 | AGTCTCTGAAAATGGAAGAAAATATAGCAGATATGGAAGGTGCA |
| | | CTCACCAGGAGCTGCAAGCCATGCAGATGGAGCTGCAGAGCCCT |
| | | GAG |
| | CCDC88A- | TGGAAATGGCACAGAAACAAAGTATGGATGAATCATTACATCTT |
| | ALK.C12A20 | GGCTGGGAACTGGAACAGATATCCAGAACTAGTGAACTTTCCGA |
| | | AGTGTACCGCCGGAAGCACCAGGAGCTGCAAGCCATGCAGATG |
| | | GAGCTGCAGAGCCCTGAG |
| | KTN1- | ACACAGTTACAGCAGTTGCTTCAGGCGGTAAACCAACAGCTCAC |
| | ALK.K43A19 | AAAGGAGAAAGAGCACTACCAGGTGTTAGTGTCACCCACCCCGG |
| | | AGCCACACCTGCCACTCTCGCTGATCCTCTCTGTGGTGACCT |
| | MSN-ALK. | CTCGAATCTCCCAGCTGGAGATGGCCCGACAGAAGAAGGAGAG |
| | M11int12A20 | TGAGGCTGTGGAGTGGCAGCAGAAGCAGGCAGCATGGGAGAA |
| | | GGCACTCATGGTTTCGTCAAATACTTACTGGAGTTCTCTCAGGAG |
| | | CTGCAAGCCATGCAGATGGAGCTGCAGAGCCCTGAG |
| | MYH9-ALK. | CTGGAGATGGACCTGAAGGACCTGGAGGCGCACATCGACTCGG |
| | M34A20del23 | CCAACAAGAACCGGGACGAAGCCATCAAACAGCTGCGGAAGCT |
| | | GCAGGTCCTGCAAGCCATGCAGATGGAGCTGCAGAGCCCTGAG |
| | EML4-ALK. | GGATGTTATTAACTGGAGGAGGGAAAGACAGAAAAATAATTCT |
| | E14A20. | GTGGGATCATGATCTGAATCCTGAAAGAGAAATAGAGCACCAG |
| | COSF1064.1 | GAGCTGCAAGCCATGCAGATGGAGCTGCAGAGCCCTGAG |
| | EML4-ALK. | GGATGTTATTAACTGGAGGAGGGAAAGACAGAAAAATAATTCT |
| | E14del36A20 | GTGGGATCATGATCTGAATCCTGAAAGAGAAATAGAGATGGAG |
| | | CTGCAGAGCCCTGAG |
| | EML4-ALK. | GACACTGTGCAGATTTTCATCCAAGTGGCACAGTGGTGGCCATA |
| | E17int17Aint | GGAACGCACTCAGGCAGGAGACAAAAACATGAAGTCAATTTTCC |
| | 19E20 | CAAAATTAAACTCATTAAAAAATGTGGAATGCTGCCAGGCCATG |
| | | TTGCAGCTGACCACCCACCTGCAGTGTACCGCCGGAAGCACCAG |
| | | GAGCTGCAAGCCATGCAGATGGAGCTGCAGAGCCCTGAG |
| | BEND5- | GTCTGAAATGAAGGAGCTCCGTGACCTTAACCGGAGGCTCCAGG |
| | ALK.B3A20 | ACGTGCTGCTCCTGCGGCTTGGCAGCGTGTACCGCCGGAAGCAC |
| | | CAGGAGCTGCAAGCCATGCAGATGGAGCTGCAGAGCCCTGAG |
| | CLTC-ALK. | TGCTTCAGAATCACTGAGAAAAGAAGAAGAACAAGCTACAGAG |
| | C31ins63A20 | ACACAACCCATTGTTTATGATGGGGTCTCGTCTGTCACCCAGGCT |
| | | GGAGTGCAGTGGCGTGATCTCGGCTCACTGCAACCTTTGTACCG |
| | | CCGGAAGCACCAGGAGCTGCAAGCCATGCAGATGGAGCTGCAG |
| | | AGCCCTGAG |
| | FN1- | CTGCAGCCTGCATCTGAGTACACCGTATCCCTCGTGGCCATAAAG |
| | ALK.F20A19 | GGCAACCAAGAGAGCCCCAAAGCCACTGGAGTCTTTACCACACT |
| | | GTCACCCACCCCGGAGCCACACCTGCCACTCTCGCTGATCCTCTC |
| | | TGTGGTGACCT |
| | KCNQ5- | GCGGGTGCAGAACTACCTGTACAACGTGCTGGAGAGACCCCGC |
| | ALK.K1A10 | GGCTGGGCGTTCATCTACCACGCTTTCGTGTTCTGGCTGCAGATG |
| | | GTCGCATGGTGGGGACAAGGATCCAGAGCCATCGTGGCTTTTGA |
| | | CAATATCTCCAT |
| | ATRNL1- | ACCACAGGAAAGCAGTGTCAAGATTGTATGCCAGGTTATTATGG |
| | ALK.A19A20 | AGATCCAACCAATGGTGGACAGTGCACAGTGTACCGCCGGAAGC |
| | | ACCAGGAGCTGCAAGCCATGCAGATGGAGCTGCAGAGCCCTGAG |
| | TRMT61 | CCAGCCTTGGAAGACTATGTAGTATTGATGAAAAGAGGGACTGC |
| | B-ALK. | CATAACATTCCCAAAGCTCCGAATGTCCTGGCTCATTCGTGGAGT |
| | T1A9 | CTTGAGGGGAAACGTGTCCTTGGTGCTAGTGGAGAACAAAACCG |
| | | GGAAGGAGCAAGGCAGG |
| | TFG- | CGGCTATGGTGCACAGCAGCCGCAGGCTCCACCTCAGCAGCCTC |
| | ALK.T7A19 | AACAGTATGGTATTCAGTATTCAGTGTCACCCACCCCGGAGCCAC |
| | | ACCTGCCACTCTCGCTGATCCTCTCTGTGGTGACCT |
| | GTF3C2- | CGTCATAAGACCGCGACCAGACAGGCGGCGCCATCTTCGAACTT |
| | ALK.G1A18 | AGACTTCCGGAAGGACTTTGGCGAGGATTATCTAAACTGCAGTC |
| | | ACTGTGAGGTAG |
| | PPFIBP1- | GTTAGTGAAATGGACAGTGAGAGACTTCAGTATGAAAAAAAGCT |
| | ALK. | TAAATCAACCAAAGTTACTACGTGCTCGGCAATTTACACATTTCA |
| | P8A20ins49 | ATTCATTCGATCCTCAGTGTACCGCCGGAAGCACCAGGAGCTGC |
| | | AAGCCATGCAGATGGAGCTGCAGAGCCCTGAG |
| | MYH9- | GGACAAGGACATGTTCCAGGAGACCATGGAGGCCATGAGGATT |
| | ALK. | ATGGGCATCCCAGAAGAGGAGCAAATGGTGCTCTCCAGGAACAT |
| | M9A6ins10 | CCCCAGGCTCCAAGATGGCCCTGCAGAGCTCCTTCACTTGTTGGA |
| | | ATGGGACAGTCCTCCAGCTTGGG |
| | DCTN1- | GCCAGCTGCTGGAGACATTGAATCAATTGAGCACACACACGCAC |
| | ALK.D29A20 | GTAGTAGACATCACTCGCACCAGCCCTGTGTACCGCCGGAAGCA |
| | | CCAGGAGCTGCAAGCCATGCAGATGGAGCTGCAGAGCCCTGAG |
| | HIP1- | AAAACTGGGAGAGCTTCGGAAAAAGCACTACGAGCTTGCTGGT |
| | ALK.H30A20 | GTTGCTGAGGGCTGGGAAGAAGTGTACCGCCGGAAGCACCAGG |
| | | AGCTGCAAGCCATGCAGATGGAGCTGCAGAGCCCTGAG |
| | CLIP1- | CAAAAGGAGGAACAGTTTAACATGCTGTCTTCTGACTTGGAGAA |
| | ALK.C13A20 | GCTGAGAGAAAACTTAGCAGTGTACCGCCGGAAGCACCAGGAG |
| | | CTGCAAGCCATGCAGATGGAGCTGCAGAGCCCTGAG |
| | BIRC6- | TGAGGAACAGGACACATTTGTTTCTGTGATTTACTGTTCTGGCAC |
| | ALK.B10A20 | AGACAGGCTGTGTGCATGCACCAAAGTGTACCGCCGGAAGCACC |
| | | AGGAGCTGCAAGCCATGCAGATGGAGCTGCAGAGCCCTGAG |
| | TERT- | ACGGCCTATTCCCCTGGTGCGGCCTGCTGCTGGATACCCGGACCC |
| | ALK.T11A5 | TGGAGGTGCAGAGCGACTACTCCAGTTGGACAGTGCTCCAGGG |
| | | AAGAATCGGGCGTCCAGACAACCCATTTCGAGTGGCCCTGGA |
| | CLIP4- | GGAGAGAGAGTGTTAGTGGTAGGACAGAGACTGGGCACCATTA |
| | ALK.C12A23 | GGTTCTTTGGGACAACAAACTTCGCTCCAGGCCCCGGTTCATCCT |
| | | GCTGGAGCTCATGGCGGGGGGAGACCTCAAGTCCTTCCTCCGAG |
| | | AGACC |
| | EML4- | GTCGAAAATACCTTCAACACCCAAATTAATACCAAAAGTTACCAA |
| | ALK. | AACTGCAGACAAGCATAAAGATGTCATCATCAACCAAGCTGACC |
| | E6ins18A20 | ACCCACCTGCAGTGTACCGCCGGAAGCACCAGGAGCTGCAAGCC |
| | | ATGCAGATGGAGCTGCAGAGCCCTGAG |
| | EML4- | GGAATGGAGATGTTCTTACTGGAGACTCAGGTGGAGTCATGCTT |
| | ALK. | ATATGGAGCAAAACTACTGTAGAGCCCACACCTGGGAAAGGACC |
| | E13ins90A20 | TAAAGATCCAGGGAGGCTTCCTGTAGGAAGTGGCCTGTGTAGTG |
| | | CTTCAAGGGCCAGG |
| | EML4-ALK. | GGATGTTATTAACTGGAGGAGGGAAAGACAGAAAAATAATTCT |
| | E14ins2del52 | GTGGGATCATGATCTGAATCCTGAAAGAGAAATAGAGGTCCCTG |
| | A20 | AGTACAAGCTGAGCAAGCTCCGCACCTCGACCATCATGACCGAC |
| | | TACAACCCCAACTACTGCTTTGCTGGCAAGACCTCCT |
| | EML4-ALK. | GGATGTTATTAACTGGAGGAGGGAAAGACAGAAAAATAATTCT |
| | E14ins124A20.1 | GTGGGATCATGATCTGAATCCTGAAAGAGAAATAGAGGGAAAG |
| | | GTTCAGAGCTCAGGGGAGGATATGGAGATCCAGGGAGGCTTCC |
| | | TGTAGGAAGTGGCCTGTGTAGTGCTTCAAGGGCCAGG |
| | EML4- | GACACTGTGCAGATTTTCATCCAAGTGGCACAGTGGTGGCCATA |
| | ALK. | GGAACGCACTCAGGCAGAGTAACAGATTCCCTGGATACCCTTTC |
| | E17ins65A20 | AGAAATTTCTTCAAATAAACAGAACCATTCTTATCCTGTGTACCG |
| | | CCGGAAGCACCAGGAGCTGCAAGCCATGCAGATGGAGCTGCAG |
| | | AGCCCTGAG |
| | EML4- | GACACTGTGCAGATTTTCATCCAAGTGGCACAGTGGTGGCCATA |
| | ALK. | GGAACGCACTCAGGCAGAGTCTTGCTCTGTCTCCCAGGCTGGAG |
| | E17ins68A20 | TGCAGTGGCAATTTACACATTTCAATTCATTCGATCCTCAGTGTAC |
| | | CGCCGGAAGCACCAGGAGCTGCAAGCCATGCAGATGGAGCTGC |
| | | AGAGCCCTGAG |
| | EML4- | GACACTGTGCAGATTTTCATCCAAGTGGCACAGTGGTGGCCATA |
| | ALK. | GGAACGCACTCAGGCAGGCCATGTTGCAGCTGACCACCCACCTG |
| | E17ins30A20_ | CAGTGTACCGCCGGAAGCACCAGGAGCTGCAAGCCATGCAGAT |
| | V8a | GGAGCTGCAGAGCCCTGAG |
| | PRKAR1A- | GCACTGCTCGACCTGAGAGACCCATGGCATTCCTCAGGGAATAC |
| | ALK. | TTTGAGAGGTTGGAGAAGACCTCCTCCATCAGTGACCTGAAGGA |
| | P2A20.NGS.2 | GGTGCCGCGGAAAAACATCACCCTCATTCGGGGTCTGGGCCATG |
| | | GCGCCTTTGGGGAGGTGTATGAAGGCCAGGTGTCCGGAATGCC |
| | | C |
| | TRAF1- | CGATGGCACTTTCCTGTGGAAGATCACCAATGTCACCAGGCGGT |
| | ALK.T6A20 | GCCATGAGTCGGCCTGTGGCAGGACCGTCAGCCTCTTCTCCCCA |
| | | GTGTACCGCCGGAAGCACCAGGAGCTGCAAGCCATGCAGATGG |
| | | AGCTGCAGAGCCCTGAG |
| | PPP4R3B- | GCACCACTTTTGACCAATACTTCAGAAGACAAATGTGAAAAGGA |
| | ALK.P9A2 | TAATATAGTTGGATCAAACAAAAACAACACAATTTGTCCCGGTCA |
| | | TAGCTCCTTGGAATCACCAACAAACATGCCTTCTCCTTCTCCTGAT |
| | | TATTTTACATGGAATCTCACCTGGATAATGAAAG |
| | DCTN1- | AGAACTAAAGCAGCGTCTGAACAGCCAGTCCAAACGCACGATTG |
| | ALK.D26A20 | AGGGACTCCGGGGCCCTCCTCCTTCAGGCATTGCTACTCTGGTCT |
| | | CTGGCATTGCTGGTGTGTACCGCCGGAAGCACCAGGAGCTGCAA |
| | | GCCATGCAGATGGAGCTGCAGAGCCCTGAG |
| | EML4- | CAAATGGCTGCAAACTAATCAGGAATCGATCGGATTGTAAGGAC |
| | ALK.E21A20 | ATTGATTGGACGACATATACCTGTGTGCTAGGATTTCAAGTATTT |
| | | GTGTACCGCCGGAAGCACCAGGAGCTGCAAGCCATGCAGATGG |
| | | AGCTGCAGAGCCCTGAG |
| | A2M- | CAGTCATCAAGCCTCTGTTGGTTGAACCTGAAGGACTAGAGAAG |
| | ALK.A22A19 | GAAACAACATTCAACTCCCTACTTTGTCCATCAGTGTCACCCACCC |
| | | CGGAGCCACACCTGCCACTCTCGCTGATCCTCTCTGTGGTGACCT |
| | TPM1- | CTGAGACTCGGGCTGAGTTTGCGGAGAGGTCAGTAACTAAATTG |
| | ALK.T8A20. | GAGAAAAGCATTGATGACTTAGAAGTGTACCGCCGGAAGCACCA |
| | NGS | GGAGCTGCAAGCCATGCAGATGGAGCTGCAGAGCCCTGAG |
| | TPR- | AAATGCAGCTTGTTGATTCCATAGTTCGTCAGCGTGATATGTACC |
| | ALK.T15A20 | GTATTTTATTGTCACAAACAACAGGAGTTGCCATTCCATTACATG |
| | | TGTACCGCCGGAAGCACCAGGAGCTGCAAGCCATGCAGATGGA |
| | | GCTGCAGAGCCCTGAG |
| | NCOA1-ALK. | CTCAAAACAGAAGCAGATGGAACCCAGCAGGTGCAACAGGTTCA |
| | N21A1.NGS | GGTGTTTGCTGACGTCCAGTGTACAGTGAATCTGGTAGGCGGCT |
| | | GTGGGGCTGCTCCAGTTCAATCTCAGCGAGCTGTTCA |
| | MEMO1- | ACAGCTAGAAGGTTGGCTTTCACAAGTACAGTCTACAAAAAGAC |
| | ALK.M2A7 | CTGCTAGAGCCATTATTGCCCCGGAAACTGCCTGTGGGTTTTTAC |
| | | TGCAACTTTGAAGATGGCTTCTGTGGCT |
| | GTF2IRD1- | CCTCTCATCCAGAACGTCCATGCCTCCAAGCGCATTCTCTTCTCCA |
| | ALK.G7A20 | TCGTCCATGACAAGTCAGTGTACCGCCGGAAGCACCAGGAGCTG |
| | | CAAGCCATGCAGATGGAGCTGCAGAGCCCTGAG |
| | HIP1- | AGCGACGCCATTGCTCATGGTGCCACCACCTGCCTCAGAGCCCCA |
| | ALK.H21A20 | CCTGAGCCTGCCGACTTGTACCGCCGGAAGCACCAGGAGCTGCA |
| | | AGCCATGCAGATGGAGCTGCAGAGCCCTGAG |
| | HIP1- | GCGTTGTGGCCTCAACCATTTCCGGCAAATCACAGATCGAAGAG |
| | ALK.H28A20 | ACAGTGTACCGCCGGAAGCACCAGGAGCTGCAAGCCATGCAGA |
| | | TGGAGCTGCAGAGCCCTGAG |
| | EML4-ALK. | GGAATGGAGATGTTCTTACTGGAGACTCAGGTGGAGTCATGCTT |
| | E13A20. | ATATGGAGCAAAACTACTGTAGAGCCCACACCTGGGAAAGGACC |
| | COSF1062.2 | TAAAGGAAGTGGCCTGTGTAGTGCTTCAAGGGCCAGG |
| | EML4- | GGATGTTATTAACTGGAGGAGGGAAAGACAGAAAAATAATTCT |
| | ALK.E14A20. | GTGGGATCATGATCTGAATCCTGAAAGAGAAATAGAGATATGCT |
| | COSF477.1 | GGATGAGCCCTGAGTACAAGCTGAGCAAGCTCCGCACCTCGACC |
| | | ATCATGACCGACTACAACCCCAACTACTGCTTTGCTGGCAAGACC |
| | | TCCT |
| | EML4-ALK. | GACACTGTGCAGATTTTCATCCAAGTGGCACAGTGGTGGCCATA |
| | E17A20. | GGAACGCACTCAGGCAGCATACTATGTATACAAGGGAGTTGCAG |
| | COSF1366.2 | AGCCCTGAGTACAAGCTGAGCAAGCTCCGCACCTCGACCATCAT |
| | | GACCGACTACAACCCCAACTACTGCTTTGCTGGCAAGACCTCCT |
| | EML4-ALK. | GACACTGTGCAGATTTTCATCCAAGTGGCACAGTGGTGGCCATA |
| | E17A20. | GGAACGCACTCAGGCAGGGAGTTGCAGAGCCCTGAGTACAAGC |
| | COSF1367.2 | TGAGCAAGCTCCGCACCTCGACCATCATGACCGACTACAACCCCA |
| | | ACTACTGCTTTGCTGGCAAGACCTCCT |
| | EML4-ALK. | CATTCCAGCTACATCACACACCTTGACTGGTCCCCAGACAACAAG |
| | E20A20. | TATATAATGTCTAACTCGGGAGACTATGAAATATTGTACTCTGAC |
| | COSF730.1 | CACCCACCTGCAGTGTACCGCCGGAAGCACCAGGAGCTGCAAGC |
| | | CATGCAGATGGAGCTGCAGAGCCCTGAG |
| | EML4-ALK. | GCGGCTTTGGCTGATGTTTTGAGGCGTCTTGCAATCTCTGAAGAT |
| | E2A20. | CATGTGGCCTCAGTGAAAAAATCAGTCTCAAGTAAAGGTTCAGA |
| | COSF479.1 | GCTCAGGGGAGGATATGGAGATCCAGGGAGGCTTCCTGTAGGA |
| | | AGTGGCCTGTGTAGTGCTTCAAGGGCCAGG |
| | EML4- | GTCGAAAATACCTTCAACACCCAAATTAATACCAAAAGTTACCAA |
| | ALK.E6A17 | AACTGCAGACAAGCATAAAGATGTCATCATCAACCAAGGCGGCA |
| | | ATGCAGCCTCAAACAATGACCCCGAAATGGATGGGGAAGATGG |
| | | GGT |
| | EML4- | GTCGAAAATACCTTCAACACCCAAATTAATACCAAAAGTTACCAA |
| | ALK.E6A18 | AACTGCAGACAAGCATAAAGATGTCATCATCAACCAAGTGATGG |
| | | AAGGCCACGGGGAAGTGAATATTAAGCATTATCTAAACTGCAGT |
| | | CACTGTGAGGTAG |
| | EML4-ALK. | GTCGAAAATACCTTCAACACCCAAATTAATACCAAAAGTTACCAA |
| | E6bA20. | AACTGCAGACAAGCATAAAGATGTCATCATCAACCAAGCAAAAA |
| | AB374362 | TGTCAACTCGCGAAAAAAACAGCCAAGTGTACCGCCGGAAGCAC |
| | | CAGGAGCTGCAAGCCATGCAGATGGAGCTGCAGAGCCCTGAG |
| | EML4- | AATTACCATGTTCATTCCTTCCGATGTTGACAACTATGATGACATC |
| | ALK.E7A20. | AGAACGGAACTGCCTCCTGAGAAGCTGAGCAAGCTCCGCACCTC |
| | NGS | GACCATCATGACCGACTACAACCCCAACTACTGCTTTGCTGGCAA |
| | | GACCTCCT |
| | ACTG2- | CAGGCTTCGCAGGAGATGATGCCCCCCGGGCTGTCTTCCCCTCCA |
| | ALK.A2A18 | TTGTGGGCCGCCCTCGCCACCAGTGATGGAAGGCCACGGGGAA |
| | | GTGAATATTAAGCATTATCTAAACTGCAGTCACTGTGAGGTAG |
| | CLTC- | TGGATTTTGCCATGCCCTATTTCATCCAGGTCATGAAGGAGTACT |
| | ALK.C31A20. | TGACAAAGGGTGAAGGTTCAGAGCTCAGGGGAGGATATGGAGA |
| | COSF470 | TCCAGGGAGGCTTCCTGTAGGAAGTGGCCTGTGTAGTGCTTCAA |
| | | GGGCCAGG |
| | FN1- | TGTCTCCACCAACAAACTTGCATCTGGAGGCAAACCCTGACACTG |
| | ALK.F23A19. | GAGTGCTCACAGTCTCCTGGGAGAGGAGCACCACCCCAGTGTCA |
| | COSF1301 | CCCACCCCGGAGCCACACCTGCCACTCTCGCTGATCCTCTCTGTG |
| | | GTGACCT |
| | RNF213- | GAAGGGAGGAACTGTTACTTCTAAAGAAAGAGAAAAGATGTGT |
| | ALK.R20A20 | TGATAGTCTCCTGAAGATGTGTGGGAACGTGAAACATCTGATAC |
| | | AAGTGTACCGCCGGAAGCACCAGGAGCTGCAAGCCATGCAGAT |
| | | GGAGCTGCAGAGCCCTGAG |
| | PPFIBP1- | GATCTTCGACAGTGCCTGAACAGGTACAAGAAAATGCAAGACAC |
| | ALK.P12A20. | GGTGGTACTGGCCCAAGGTAAAAAAGTGTACCGCCGGAAGCAC |
| | COSF1461 | CAGGAGCTGCAAGCCATGCAGATGGAGCTGCAGAGCCCTGAG |
| | SQSTM1- | CTTCTGGTCCATCGGAGGATCCGAGTGTGAATTTCCTGAAGAAC |
| | ALK.S5A20. | GTTGGGGAGAGTGTGGCAGCTGCCCTTAGCCCTCTGGTGTACCG |
| | COSF1051 | CCGGAAGCACCAGGAGCTGCAAGCCATGCAGATGGAGCTGCAG |
| | | AGCCCTGAG |
| | STRN- | AGGAAAGAGCCAAATACCACAAGTTGAAATACGGGACAGAATT |
| | ALK.S3A20. | GAATCAGGGAGATATGAAGCCTCCAAGCTATGATTCTGTGTACC |
| | COSF1430 | GCCGGAAGCACCAGGAGCTGCAAGCCATGCAGATGGAGCTGCA |
| | | GAGCCCTGAG |
| | TPM4- | CTGACAAACTGAAAGAGGCTGAGACCCGTGCTGAATTTGCAGAG |
| | ALK.T7A20. | AGAACGGTTGCAAAACTGGAAAAGTGTACCGCCGGAAGCACCA |
| | COSF441 | GGAGCTGCAAGCCATGCAGATGGAGCTGCAGAGCCCTGAG |
| | VCL- | CTGTGAAAGCTGCCTCTGATGAATTGAGCAAAACCATCTCCCCGA |
| | ALK.V16A20. | TGGTGATGGATGCAAAAGCTGTGGCTGGAAACATTTCCGACCCT |
| | COSF1057 | GTGTACCGCCGGAAGCACCAGGAGCTGCAAGCCATGCAGATGG |
| | | AGCTGCAGAGCCCTGAG |
| | KIF5B- | GAGCAGCTGAGATGATGGCATCTTTACTAAAAGACCTTGCAGAA |
| | ALK.K15A20. | ATAGGAATTGCTGTGGGAAATAATGATGTAAAGTGTACCGCCGG |
| | COSF1381 | AAGCACCAGGAGCTGCAAGCCATGCAGATGGAGCTGCAGAGCC |
| | | CTGAG |
| | KIF5B- | GAGCAGCTGAGATGATGGCATCTTTACTAAAAGACCTTGCAGAA |
| | ALK.K15A20. | ATAGGAATTGCTGTGGGAAATAATGATGTAAAGCACCAGGAGCT |
| | COSF1060.1 | GCAAGCCATGCAGATGGAGCTGCAGAGCCCTGAG |
| | KIF5B- | AAAGAAAAGACAGTTGGAGGAATCTGTCGATGCCCTCAGTGAA |
| | ALK.K17A20. | GAACTAGTCCAGCTTCGAGCACAAGTGTACCGCCGGAAGCACCA |
| | COSF1257 | GGAGCTGCAAGCCATGCAGATGGAGCTGCAGAGCCCTGAG |
| | KIF5B- | ATCGCAAACGCTATCAGCAAGAAGTAGATCGCATAAAGGAAGCA |
| | ALK.K24A20. | GTCAGGTCAAAGAATATGGCCAGAAGAGGGCATTCTGCACAGAT |
| | COSF1058 | TGTGTACCGCCGGAAGCACCAGGAGCTGCAAGCCATGCAGATG |
| | | GAGCTGCAGAGCCCTGAG |
| | TFG- | CCTCCTCAGCAGCTCACCCACCAGGCGTTCAGCCACAGCAGCCAC |
| | ALK.T6A20. | CATATACAGGAGCTCAGACTCAAGCAGGTCAGATTGAAGTGTAC |
| | COSF428 | CGCCGGAAGCACCAGGAGCTGCAAGCCATGCAGATGGAGCTGC |
| | | AGAGCCCTGAG |
| | TFG- | AGTGAATCGTTTATTGGATAGCTTGGAACCACCTGGAGAACCAG |
| | ALK.T4A20. | GACCTTCCACCAATATTCCTGAAAATGTGTACCGCCGGAAGCACC |
| | COSF424 | AGGAGCTGCAAGCCATGCAGATGGAGCTGCAGAGCCCTGAG |
| | TFG- | AAAAATGTTATGTCAGCGTTTGGCTTAACAGATGATCAGGTTTCA |
| | ALK.T5A20. | GTGTACCGCCGGAAGCACCAGGAGCTGCAAGCCATGCAGATGG |
| | COSF426 | AGCTGCAGAGCCCTGAG |
| | TPM3- | CAGAGACCCGTGCTGAGTTTGCTGAGAGATCGGTAGCCAAGCTG |
| | ALK.T7A20. | GAAAAGACAATTGATGACCTGGAAGTGTACCGCCGGAAGCACCA |
| | COSF439 | GGAGCTGCAAGCCATGCAGATGGAGCTGCAGAGCCCTGAG |
| | SEC31A- | CAAATGCTGCTGGTCAGCTTCCCACATCTCCAGGTCATATGCACA |
| | ALK.S21A20. | CCCAGGTACCACCTTATCCACAGCCACAGCTGTACCGCCGGAAG |
| | COSF460 | CACCAGGAGCTGCAAGCCATGCAGATGGAGCTGCAGAGCCCTG |
| | | AG |
| | SEC31A- | GCTCCACCATCATCTTCAGCTTATGCACTGCCTCCTGGAACAACA |
| | ALK.S22A20. | GGTACACTGCCTGCTGCCAGTGAGCTGCCTGCGTCCCAAAGAAC |
| | COSF459 | AGTGTACCGCCGGAAGCACCAGGAGCTGCAAGCCATGCAGATG |
| | | GAGCTGCAGAGCCCTGAG |
| | RANBP2- | CATCGTTGGCCCACAGAGAATTATGGACCAGACTCAGTGCCTGA |
| | ALK.R18 | TGGATATCAGGGGTCACAGACATTTCATGGGGCTCCACTAACAG |
| | A20.COSF415 | TGTACCGCCGGAAGCACCAGGAGCTGCAAGCCATGCAGATGGA |
| | | GCTGCAGAGCCCTGAG |
| | NPM1- | GGGCTTTGAAATAACACCACCAGTGGTCTTAAGGTTGAAGTGTG |
| | ALK.N4A20. | GTTCAGGGCCAGTGCATATTAGTGGACAGCACTTAGTAGTGTAC |
| | COSF198 | CGCCGGAAGCACCAGGAGCTGCAAGCCATGCAGATGGAGCTGC |
| | | AGAGCCCTGAG |
| | MSN- | CTCGAATCTCCCAGCTGGAGATGGCCCGACAGAAGAAGGAGAG |
| | ALK.M11A20. | TGAGGCTGTGGAGTGGCAGCAGAAGCAGGAGCTGCAAGCCATG |
| | COSF421 | CAGATGGAGCTGCAGAGCCCTGAG |
| | KLC1- | CAAGCAGAAACACTGTACAAAGAGATTCTCACTCGTGCACATGA |
| | ALK.K9A20. | AAGGGAGTTTGGTTCTGTAGATGTGTACCGCCGGAAGCACCAGG |
| | COSF1276 | AGCTGCAAGCCATGCAGATGGAGCTGCAGAGCCCTGAG |
| | CLTC- | TGCTTCAGAATCACTGAGAAAAGAAGAAGAACAAGCTACAGAG |
| | ALK.C31A20. | ACACAACCCATTGTTTATGTGTACCGCCGGAAGCACCAGGAGCT |
| | COSF434 | GCAAGCCATGCAGATGGAGCTGCAGAGCCCTGAG |
| | EML4- | GGAATGGAGATGTTCTTACTGGAGACTCAGGTGGAGTCATGCTT |
| | ALK.E13A20. | ATATGGAGCAAAACTACTGTAGAGCCCACACCTGGGAAAGGACC |
| | COSF408.1 | TAAAGTGTACCGCCGGAAGCACCAGGAGCTGCAAGCCATGCAG |
| | | ATGGAGCTGCAGAGCCCTGAG |
| | EML4- | TCCTGAAAGAGAAATAGAGGTTCCTGATCAGTATGGCACAATCA |
| | ALK.E15A20. | GAGCTGTAGCAGAAGGAAAGGCAGATCAATTTTTAGTAGGCAA |
| | COSF413.1 | GCTCCGCACCTCGACCATCATGACCGACTACAACCCCAACTACTG |
| | | CTTTGCTGGCAAGACCTCCT |
| | EML4- | TGGATGCAGAAACCAGAGATCTAGTTTCTATCCACACAGACGGG |
| | ALK.E18A20. | AATGAACAGCTCTCTGTGATGCGCTACTCAATAGTGTACCGCCGG |
| | COSF487.1 | AAGCACCAGGAGCTGCAAGCCATGCAGATGGAGCTGCAGAGCC |
| | | CTGAG |
| | EML4- | CATTCCAGCTACATCACACACCTTGACTGGTCCCCAGACAACAAG |
| | ALK.E20A20. | TATATAATGTCTAACTCGGGAGACTATGAAATATTGTACTTGTAC |
| | COSF409.1 | CGCCGGAAGCACCAGGAGCTGCAAGCCATGCAGATGGAGCTGC |
| | | AGAGCCCTGAG |
| | EML4- | GCGGCTTTGGCTGATGTTTTGAGGCGTCTTGCAATCTCTGAAGAT |
| | ALK.E2A20. | CATGTGGCCTCAGTGAAAAAATCAGTCTCAAGTAAAGTGTACCG |
| | COSF478.1 | CCGGAAGCACCAGGAGCTGCAAGCCATGCAGATGGAGCTGCAG |
| | | AGCCCTGAG |
| | EML4- | GTCGAAAATACCTTCAACACCCAAATTAATACCAAAAGTTACCAA |
| | ALK.E6A19. | AACTGCAGACAAGCATAAAGATGTCATCATCAACCAAGTGTCAC |
| | COSF1296.1 | CCACCCCGGAGCCACACCTGCCACTCTCGCTGATCCTCTCTGTGG |
| | | TGACCT |
| | EML4- | GTCGAAAATACCTTCAACACCCAAATTAATACCAAAAGTTACCAA |
| | ALK.E6aA20. | AACTGCAGACAAGCATAAAGATGTCATCATCAACCAAGTGTACC |
| | AB374361 | GCCGGAAGCACCAGGAGCTGCAAGCCATGCAGATGGAGCTGCA |
| | | GAGCCCTGAG |
| | ATIC- | GGAAACAGTACAGCAAAGGCGTATCTCAGATGCCCTTGAGATAT |
| | ALK.A7A20. | GGAATGAACCCACATCAGACCCCTGCCCAGCTGTACACACTGCA |
| | COSF444 | GCCCAAGCTTCCCATCACAGTGTACCGCCGGAAGCACCAGGAGC |
| | | TGCAAGCCATGCAGATGGAGCTGCAGAGCCCTGAG |
| | CARS- | CACAGTCATGCCCTACCTTCAGGTGTTATCAGAATTCCGAGAAGG |
| | ALK.C17A20. | AGTGCGGAAGATTGCCCGAGAGCAAAAAGTGTACCGCCGGAAG |
| | COSF437 | CACCAGGAGCTGCAAGCCATGCAGATGGAGCTGCAGAGCCCTG |
| | | AG |
| ARMT1 | ESR1- | GCCCTACTACCTGGAGAACGAGCCCAGCGGCTACACGGTGCGCG |
| | ARMT1.E3A4 | AGGCCGGCCCGCCGGCATTCTACAGTCCACCAATCGATTACTTTG |
| | | ATGTATTTAAAGAATCAAAAGAGCAAAATTTCTATGGGTCACAG |
| | | GA |
| ATAD5 | NF1- | CTGTTTGTTCAGAAGACAATGTTGATGTTCATGATATAGAATTGT |
| | ATAD5.N5A11 | TACAGTATATCAATGTGGATTGTGCAAAATTAAAACGACTCCTGA |
| | | AGGATTCTGGAACTGAAGACATGCTTTGGACAGAAAAGTATCAA |
| | | CCTCAGACTGCCAGTG |
| ATG7 | ATG7- | CTAGCCAAGGTGTTTAATTCTTCACATTCCTTCTTAGAAGACTTGA |
| | BRAF.A18B9 | CTGGTCTTACATTGCTGCATCAAGAAACCCAAGCTGCTGAGGACT |
| | | TGATTAGAGACCAAGGATTTCGTGGTGATGGAGGATCAACCACA |
| | | GGTTT |
| ATIC | ATIC- | GGAAACAGTACAGCAAAGGCGTATCTCAGATGCCCTTGAGATAT |
| | ALK.A7A20. | GGAATGAACCCACATCAGACCCCTGCCCAGCTGTACACACTGCA |
| | COSF444 | GCCCAAGCTTCCCATCACAGTGTACCGCCGGAAGCACCAGGAGC |
| | | TGCAAGCCATGCAGATGGAGCTGCAGAGCCCTGAG |
| AXL | AXL- | ACATGGATGAGGGTGGAGGTTATCCTGAACCCCCTGGAGCTGCA |
| | MBIP.A20M4.1 | GGAGGAGCTGACCCCCCAACCCAGCCAGACCCTAAGGATTCCTG |
| | | TAGCTGCCTCACTGCGGCTGAGATTGACAGACGAATATCTGCATT |
| | | TATTGAAAGAAAGCAAGCTGAAATCAA |
| BIRC6 | BIRC6- | TGAGGAACAGGACACATTTGTTTCTGTGATTTACTGTTCTGGCAC |
| | ALK.B10A20 | AGACAGGCTGTGTGCATGCACCAAAGTGTACCGCCGGAAGCACC |
| | | AGGAGCTGCAAGCCATGCAGATGGAGCTGCAGAGCCCTGAG |
| BRD3 | BRD3- | CACTTTGCGGGAACTGGAGAGATATGTCAAGTCTTGTTTACAGA |
| | NUTM1.B10N2 | AAAAGCAAAGGAAACCGTTCTCATCTGCATTGCCGGGACCGGAT |
| | | ATGAGCATGAAACCTAGTGCCGCCCCGTCTCCATCCCCTGCACTT |
| | | CCCTTTCTCCCACCAAC |
| BRD4 | BRD4- | GTCACAGTTCCAGAGCCTGACCCACCAGTCTCCACCCCAGCAAAA |
| | NUTM1.B15N2 | CGTCCAGCCTAAGAAACAGCATCTGCATTGCCGGGACCGGATAT |
| | | GAGCATGAAACCTAGTGCCGCCCCGTCTCCATCCCCTGCACTTCC |
| | | CTTTCTCCCACCAAC |
| | BRD4- | CTCGTCCTCAGAGTCGGAGAGCTCCAGTGAGTCCAGCTCCTCTGA |
| | NUTM1.B11N2 | CAGCGAAGACTCCGAAACAGCATCTGCATTGCCGGGACCGGATA |
| | | TGAGCATGAAACCTAGTGCCGCCCCGTCTCCATCCCCTGCACTTC |
| | | CCTTTCTCCCACCAAC |
| | BRD4- | GCCAAGCCTCAGCAAGTCATCCAGCACCACCATTCACCCCGGCAC |
| | NUTM1. | CACAAGTCGGACCCCTACTCAACCGGTGACCGCTCCAAAATTTCC |
| | B14N2del585 | AAGGACGTTTATGAGAACTTCCGTCAGTGGCAGCGTTACAAAG |
| CAPRIN1 | CAPRIN1- | CAGAATGGGCTGTGTGAGGAAGAAGAGGCAGCCTCAGCACCTG |
| | PDGFRB.C7P11 | CAGTTGAAGACCAGGTACCTGAAGCTGCCTTGCCCTTTAAGGTG |
| | | GTGGTGATCTCAGCCATCCTGGCCCTGGTGGTGCTCACCATCATC |
| | | TCCCTTATCATCCTCATCATGCTTTGGC |
| CCAR2 | FGFR2- | CTCCCAGAGACCAACGTTCAAGCAGTTGGTAGAAGACTTGGATC |
| | CCAR2.F17C4 | GAATTCTCACTCTCACAACCAATGAGGGTGGGGAGAAACAGCGG |
| | | GTCTTCACTGGTATTGTTACCAGCTTGCATGACTACTTTGGGGTT |
| | | GTGGATGAAGAGG |
| CCDC6 | CCDC6- | GGAGACCTACAAACTGAAGTGCAAGGCACTGCAGGAGGAGAAC |
| | BRAF.C1B9 | CGCGACCTGCGCAAAGCCAGCGTGACCATCGACTTGATTAGAGA |
| | | CCAAGGATTTCGTGGTGATGGAGGATCAACCACAGGTTT |
| | FGFR2- | CTCCCAGAGACCAACGTTCAAGCAGTTGGTAGAAGACTTGGATC |
| | CCDC6.F17C1 | GAATTCTCACTCTCACAACCAATGAGTCGCCGCCGCTCCGAGTCT |
| | | GCGCCCTGGTGCCAGGCGCTCAGCTCGGCGCTCCCCTGTGCTCG |
| | | CCCGGCGCCCACTCATTCGCAGCCCG |
| | CCDC6- | GGAGACCTACAAACTGAAGTGCAAGGCACTGCAGGAGGAGAAC |
| | RET.C1R12. | CGCGACCTGCGCAAAGCCAGCGTGACCATCGAGGATCCAAAGTG |
| | COSF1271 | GGAATTCCCTCGGAAGAACTTGGTTCTTGGAAAAACTCTAGGAG |
| | | AAGGCGAATTTGG |
| | CCDC6- | TGGTTTCACGCCACCAACTTCACTGACTAGAGCTGGAATGTCTTA |
| | RET.C8R11. | TTACAATTCCCCGGGTCTTCACGTGCAGCACATGGGAACATCCCA |
| | COSF1518 | TGGTATCACAATCTCCTCAGCTGAGATGACCTTCCGGAGGCCCGC |
| | | CCAGGCCTTCCCGGTCAGCTACTCCTCTTCCGG |
| | CCDC6- | GGAGACCTACAAACTGAAGTGCAAGGCACTGCAGGAGGAGAAC |
| | RET.C1R13 | CGCGACCTGCGCAAAGCCAGCGTGACCATCGAGTGAGCTGCGA |
| | | GACCTGCTGTCAGAGTTCAACGTCCTGAAG |
| | CCDC6- | AGGAGAAAGAAACCCTTGCTGTAAATTATGAGAAAGAAGAAGA |
| | RET.C2R12.1 | ATTCCCTCGGAAGAACTTGGTTCTTGGAAAAACTCTAGGAGAAG |
| | | GCGAATTTGG |
| | CCDC6- | TGGTTTCACGCCACCAACTTCACTGACTAGAGCTGGAATGTCTTA |
| | RET.C8R11 | TTACAATTCCCCGGGTCTTCACGTGCAGCACATGGGAACATCCCA |
| | | TGGTATCACAAGTTTGCCCACAAGCCACCCATCTCCTCAGCTGAG |
| | | ATGACCTTCCGGAGGCCCGCCCAGGCCTTCCCGGTCAGCTACTCC |
| | | TCTTCCGG |
| | CCDC6- | TGGTTTCACGCCACCAACTTCACTGACTAGAGCTGGAATGTCTTA |
| | RET.C8R12 | TTACACCACGGTGGCCGTGAAGATGCTGAAAGAGAACGCCTCCC |
| | | CGAGTGAGCTGCGAGACCTGCTGTCAGAGTTCAACGTCCTGAAG |
| | CCDC6- | GGAGACCTACAAACTGAAGTGCAAGGCACTGCAGGAGGAGAAC |
| | RET.C1R12 | CGCGACCTGCGCAAAGTGGGAATTCCCTCGGAAGAACTTGGTTC |
| | | TTGGAAAAACTCTAGGAGAAGGCGAATTTGG |
| | CCDC6- | GGAGACCTACAAACTGAAGTGCAAGGCACTGCAGGAGGAGAAC |
| | RET.C1R11 | CGCGACCTGCGCAAAGCCACCCATCTCCTCAGCTGAGATGACCTT |
| | | CCGGAGGCCCGCCCAGGCCTTCCCGGTCAGCTACTCCTCTTCCGG |
| | CCDC6- | GGAGACCTACAAACTGAAGTGCAAGGCACTGCAGGAGGAGAAC |
| | RET.C1R11.1 | CGCGACCTGCGCAAAGCCAGCGTGACCATCTTTGCCCACAAGCC |
| | | ACCCATCTCCTCAGCTGAGATGACCTTCCGGAGGCCCGCCCAGG |
| | | CCTTCCCGGTCAGCTACTCCTCTTCCGG |
| | CCDC6- | AGGAGAAAGAAACCCTTGCTGTAAATTATGAGAAAGAAGAAGA |
| | RET.C2R11 | ATTCCTCACTAATGAGCTCTCCAGAAAATTGATGCAGATCCACTG |
| | | TGCGACGAGCTGTGCCGCACGGTGATCGCAGCCGCTGT |
| | CCDC6- | CGGCTGAAGAAGCAACTGAGAGCTGCTCAGTTACAGCAGTCTTG |
| | RET.C5ins16R11 | CTGTGTTGCCCACAAGTTTGCCCACAAGCCACCCATCTCCTCAGC |
| | | TGAGATGACCTTCCGGAGGCCCGCCCAGGCCTTCCCGGTCAGCT |
| | | ACTCCTCTTCCGG |
| | CCDC6- | GGAGACCTACAAACTGAAGTGCAAGGCACTGCAGGAGGAGAAC |
| | RET.C1R9 | CGCGACCTGCGCAAAGCCAGCGTGACCATCGGATCACCAGGAAC |
| | | TTCTCCACCTGCTCTCCCAGCACCAAGACCTG |
| | CCDC6- | CGGCTGAAGAAGCAACTGAGAGCTGCTCAGTTACAGCTCTGGCA |
| | ROS1.C5R35.1 | TAGAAGATTAAAGAATCAAAAAAGTGCCAAGGAAGGGGTGACA |
| | | GTGCTTATAAACGAAGACAAAGAGTTGGCTGA |
| | CCDC6- | CTTACACACCTTCTCCGAGTTCAAGCAGGCCTATATCACCTGCCTT |
| | PDGFRB.C7P11 | GCCCTTTAAGGTGGTGGTGATCTCAGCCATCCTGGCCCTGGTGG |
| | | TGCTCACCATCATCTCCCTTATCATCCTCATCATGCTTTGGC |
| | FGFR2- | CTCCCAGAGACCAACGTTCAAGCAGTTGGTAGAAGACTTGGATC |
| | CCDC6.F17C2 | GAATTCTCACTCTCACAACCAATGAGCAAGCCAGGGCTGAGCAG |
| | | GAAGAAGAATTCATTAGTAACACTTTATTCAAGAAAATTCAGGCT |
| | | TTGCAGAAGGAGAAAGAAACCC |
| CDK5RAP2 | CDK5RAP2- | AGAAAGTACCAATCAGAAGGACGTGTTGCTTCAGGCCTGGAGCC |
| | PDGFRA. | CTCCCTTCTCAAAGAGAACCCTGCGGGCAACTTATGACTCAAGAT |
| | C13ins40P12 | GGGAGTTTCCAAGAGATGGACTAGTGCTTGGTCGGGTCTTGGGG |
| | | TCTGGAGCGTTTGGGAAGGTGGTTGAAGGAA |
| CHD9 | CHD9- | GCTCGGAGTTGGCATTCATCATTTTCTAATCATCAGCATTTACATG |
| | RAD51B.C2R8 | ACAGAAATCACCTATGTTTACAGCGACAGGTTATCTTGACGAATC |
| | | AGATTACAACCCATCTGAGTGGAGCCCTGGCTTCTCAGGCAGAC |
| | | CTGGTGTCTCCAGCTG |
| CIT | FGFR2- | CTCCCAGAGACCAACGTTCAAGCAGTTGGTAGAAGACTTGGATC |
| | CIT.F17C23 | GAATTCTCACTCTCACAACCAATGAGGCACATAGAGATGAAATCC |
| | | AGCGCAAATTTGATGCTCTTCGTAACAGCTGTACTGTAATCACAG |
| | | ACCTGGAGGAGCA |
| CTNNB1 | CTNNB1- | GGAGGAAGGTCTGAGGAGCAGCTTCAGTCCCCGCCGAGCCGCC |
| | FGFR2.C1F10 | ACCGCAGGTCGAGGACGGTCGGACTCCCGCGGCGGGAGGAGCC |
| | | TGTTCCCCTGAGGTTTCGGCTGAGTCCAGCTCCTCCATGAACTCC |
| | | AACACCC |
| CUL1 | CUL1- | TCTTGCAGCAGAACCCAGTTACTGAATATATGAAAAAGGACTTG |
| | BRAF.C7B9 | ATTAGAGACCAAGGATTTCGTGGTGATGGAGGATCAACCACAGG |
| | | TTT |
| EBF1 | EBF1- | CCAGTCGTCAGACCCCAGACCTCCCCACCTCCCACCTGCACCAGC |
| | PDGFRB.E15P11 | ACCAACGGGAACAGCCTGCAAGCCTTGCCCTTTAAGGTGGTGGT |
| | | GATCTCAGCCATCCTGGCCCTGGTGGTGCTCACCATCATCTCCCTT |
| | | ATCATCCTCATCATGCTTTGGC |
| | EBF1- | CCATCGATTATGGTTTCCAGAGGTTACAGAAGGTCATTCCTCGGC |
| | PDGFRB.E11P11 | ACCCTGGTGACCCTGAGCGTTTGCCAAAGCCTTGCCCTTTAAGGT |
| | | GGTGGTGATCTCAGCCATCCTGGCCCTGGTGGTGCTCACCATCAT |
| | | CTCCCTTATCATCCTCATCATGCTTTGGC |
| | EBF1- | CTCTGCCGCAATGTCCAATTTGGGCGGCTCCCCCACCTTCCTCAA |
| | PDGFRB.E14P11 | CGGCTCAGCTGCCAACTCCCCCTATGCCACCTTGCCCTTTAAGGT |
| | | GGTGGTGATCTCAGCCATCCTGGCCCTGGTGGTGCTCACCATCAT |
| | | CTCCCTTATCATCCTCATCATGCTTTGGC |
| | EBF1- | CTCTGCCGCAATGTCCAATTTGGGCGGCTCCCCCACCTTCCTCAA |
| | JAK2.E14J17 | CGGCTCAGCTGCCAACTCCCCCTATGCCATTCTTCAGGAGAGAAT |
| | | ACCATGGGTACCACCTGAATGCATTGAAAATCCTAAAAATTTAAA |
| | | TTTGGCAACAGACAAATGGAGTTTTGG |
| EIF3E | EIF3E- | CTCGCATCGCGCACTTTTTGGATCGGCATCTAGTCTTTCCGCTTCT |
| | RSPO2.E1R2. | TGAATTTCTCTCTGTAAAGGAGGTTCGTGGCGGAGAGATGCTGA |
| | COSF1307 | TCGCGCTGAACTGACCGGTGCGGCCCGGGGGTGAGTGGCGAGT |
| | | CTCCCT |
| | EIF3E- | CTCGCATCGCGCACTTTTTGGATCGGCATCTAGTCTTTCCGCTTCT |
| | RAD51B.E1R5 | TGAATTTCTCTCTGTAAAGGAGATTACAGGTCCACCAGGTTGTGG |
| | | AAAAACTCAGTTTTGTATAATGATGAGCATTTTGGCTACATTACC |
| | | CACCAACATGGGAG |
| | EIF3E- | CTCGCATCGCGCACTTTTTGGATCGGCATCTAGTCTTTCCGCTTCT |
| | RSPO2.E1R3. | TGAATTTCTCTCTGTAAAGGAGCTAGTTATGTATCAAATCCCATTT |
| | COSF1309 | GCAAGGGTTGTTTGTCTTGTTCAAAGGACAATGGGTGTAGCCGA |
| | | TGTCAACAGAAGTT |
| | EIF3E- | CTGCAACCTCTGCCTCCTTAGTTCAAGCGATTCTCCTGCCTCAGCC |
| | RSPO2. | TCCTGAGTAGCTGGTACTACAGGTTCGTGGCGGAGAGATGCTGA |
| | E1ins351R2 | TCGCGCTGAACTGACCGGTGCGGCCCGGGGGTGAGTGGCGAGT |
| | | CTCCCT |
| HIP1 | HIP1- | AAAACTGGGAGAGCTTCGGAAAAAGCACTACGAGCTTGCTGGT |
| | ALK.H30A20 | GTTGCTGAGGGCTGGGAAGAAGTGTACCGCCGGAAGCACCAGG |
| | | AGCTGCAAGCCATGCAGATGGAGCTGCAGAGCCCTGAG |
| | HIP1- | AGCGACGCCATTGCTCATGGTGCCACCACCTGCCTCAGAGCCCCA |
| | ALK.H21A20 | CCTGAGCCTGCCGACTTGTACCGCCGGAAGCACCAGGAGCTGCA |
| | | AGCCATGCAGATGGAGCTGCAGAGCCCTGAG |
| | HIP1- | GCGTTGTGGCCTCAACCATTTCCGGCAAATCACAGATCGAAGAG |
| | ALK.H28A20 | ACAGTGTACCGCCGGAAGCACCAGGAGCTGCAAGCCATGCAGA |
| | | TGGAGCTGCAGAGCCCTGAG |
| | HIP1- | AAAACTGGGAGAGCTTCGGAAAAAGCACTACGAGCTTGCTGGT |
| | PDGFRB.H30P11 | GTTGCTGAGGGCTGGGAAGAAGCCTTGCCCTTTAAGGTGGTGGT |
| | | GATCTCAGCCATCCTGGCCCTGGTGGTGCTCACCATCATCTCCCTT |
| | | ATCATCCTCATCATGCTTTGGC |
| HMGA2 | HMGA2- | CTAAAGCAGCTCAAAAGAAAGCAGAAGCCACTGGAGAAAAACG |
| | RAD51B.H3R11 | GCCAAGAGGCAGACCTAGGAAATGGAGACAACATTTTGCTCTGT |
| | | CACCCAAGCTGAACTGAACTGGGCTCCAGAAATCCTCCCACCTCA |
| | | GCCTCCTGAGCAGCTAGGACTACAGATGTGCCACCA |
| | HMGA2- | CTAAAGCAGCTCAAAAGAAAGCAGAAGCCACTGGAGAAAAACG |
| | RAD51B. | GCCAAGAGGCAGACCTAGGAAATGGGTTATCTTGACGAATCAGA |
| | H3R8.COSF981 | TTACAACCCATCTGAGTGGAGCCCTGGCTTCTCAGGCAGACCTG |
| | | GTGTCTCCAGCTG |
| IRF2BP2 | IRF2BP2- | GGCCCTTCGAGAGCAAGTTTAAGAAGGAGCCGGCCCTGACTGCA |
| | NTRK1.\|1N10.1 | GACACTAACAGCACATCTGGAGACCCGGTGGAGAAGAAGGACG |
| | | AAACACCTTTTGGGGTCTCG |
| NOTCH1 | NOTCH1- | TGACCTGCGCATGTCTGCCATGGCCCCCACACCGCCCCAGGGTG |
| | GABBR2. | AGGTTGACGCCGACTGCATGGACGTCAATGTCCGCGGGCCTGCC |
| | N30G14. | GGACCCAGCAGGACGGGATATCTCCATCCGCCCTCTCCTGGAGC |
| | COSF1178 | ACTGTGAGAACACCCATATGACCATCTGGCTTGGCATCG |
| | SEC16A- | CTGAGGTGTCTGTGCTCGTCGCCAGCGTCGGGGGGCTTTCGCC |
| | NOTCH1.S1N27 | CGCGGCTCCTGAGGGATCGGTCTCAGCCGCGCGGCTCCATCGTC |
| | | TACCTGGAGATTGACAACCGGCAGTGT |
| | SEC16A- | CTGAGGTGTCTGTGCTCGTCGCCAGCGTCGGGTGGGCTTTCGCC |
| | NOTCH1.S1N28 | CGCGGCTCCTGAGGGATCGGTCTCAGCCGCGCGGGTGAGACCG |
| | | TGGAGCCGCCCCCGCCGGCGCAGCTGCACTTCATGTACGTGGCG |
| | | GC |
| | MIR143HG- | GCTGGGTCTAATTAGTTGAGAAGCAGTGACACCCCCAACCACTC |
| | NOTCH1.M1N27 | CCCAAACAGGCTGGCTCCCGTCTCCAGGCCCCAAGGAGCCACAC |
| | | CTGGACCAGACCCCAGGAAAGCTCCATCGTCTACCTGGAGATTG |
| | | ACAACCGGCAGTGT |
| | NOTCH1- | CGGTGAGACCTGCCTGAATGGCGGGAAGTGTGAAGCGGCCAAT |
| | NUP214.N2N25 | GGCACGGAGGCCTGCGTTTCTTCAGTGCCCTACTCCACAGCCAAA |
| | | ACACCTCACCCAGTGTTGACCCCAGTGGCTGCTAACCAAGCCAA |
| | | GCAGGGGTCTCTAATAAA |
| | NOTCH1- | ACTGTGAGGACCTGGTGGACGAGTGCTCACCCAGCCCCTGCCAG |
| | SDCCAG3. | AACGGGGCCACCTGCACGGACTACCTGGGCGGCTACTCCTGCAA |
| | N21S5 | GCTGAAAGATGAAAATTCTAAGCTGAGAAGAAAGCTGAATGAG |
| | | G |
| | NOTCH1- | CGGTGAGACCTGCCTGAATGGCGGGAAGTGTGAAGCGGCCAAT |
| | SNHG7.N2S4 | GGCACGGAGGCCTGCGTATGCAGAGGCCAGGATGTGGGCCCAG |
| | | CCCTGTGCCAGGAGGCTGGCTGGAATAAAGAGTAACAAACCCCC |
| | | TTGGAGGACTCTCCTGCCG |
| NOTCH4 | NSD1- | GGGTCAAAGATCCTTGCATCTAATAGTATCATCTGCCCTAATCAC |
| | NOTCH4.N14N18 | TTTACCCCTAGGCGGGGCTGCCGAAATCATGAGCATGTTAATGTT |
| | | AGCTGGTGCTTTGTGTGCTCAGAAGGCATAGACGTCTCTTCCCTT |
| | | TGCCACAATGGAGGC |
| NPM1 | NPM1- | GGGCTTTGAAATAACACCACCAGTGGTCTTAAGGTTGAAGTGTG |
| | ALK.N4A20. | GTTCAGGGCCAGTGCATATTAGTGGACAGCACTTAGTAGTGTAC |
| | COSF198 | CGCCGGAAGCACCAGGAGCTGCAAGCCATGCAGATGGAGCTGC |
| | | AGAGCCCTGAG |
| OFD1 | FGFR2- | CTCCCAGAGACCAACGTTCAAGCAGTTGGTAGAAGACTTGGATC |
| | OFD1.F17O3 | GAATTCTCACTCTCACAACCAATGAGACACAACTTCGAAACCAGC |
| | | TAATTCATGAGTTGATGCACCCTGTATTGAG |
| | OFD1- | AATCTGCTCACAGTGAAAATCCTTTAGAGAAATACATGAAAATCA |
| | JAK2.O21J13 | TCCAGCAGGAGCAAGACCAGGAGTCGGCAGATAAGAATGAAAG |
| | | CCTTGGCCAAGGCACTTTTACAAAGATTTTTAAAGGCGTACGAAG |
| | | AGAAGTAGGA |
| TACC1 | FGFR1- | CCCTCACAGAGACCCACCTTCAAGCAGCTGGTGGAAGACCTGGA |
| | TACC1.F17T7. | CCGCATCGTGGCCTTGACCTCCAACCAGGGGCTGCTGGAGTCCT |
| | COSF1362 | CTGCAGAGAAGGCCCCTGTGTCGGTGTCCTGTGGAGGTGAG |
| | FGFR1- | TCCGTCCCTGTCCCCTTTCCTGCTGGCAGGAGCCGGCTGCCTACC |
| | TACC1.F18T7 | AGGGGCCTGGGCTGCTGGAGTCCTCTGCAGAGAAGGCCCCTGT |
| | | GTCGGTGTCCTGTGGAGGTGAG |
| TACC3 | FGFR2- | CTCCCAGAGACCAACGTTCAAGCAGTTGGTAGAAGACTTGGATC |
| | TACC3.F17T11 | GAATTCTCACTCTCACAACCAATGAGGTAAAGGCGACACAGGAG |
| | | GAGAACCGGGAGCTGAGGAGCAGGTGTGAGGAGCTCCACGGG |
| | | AAGAACCTGGAACTGGGGAAGATCATGGA |
| | FGFR3- | GGACCTGGACCGTGTCCTTACCGTGACGTCCACCGACGTGCCAG |
| | TACC3.F17T10. | GCCCACCCCCAGGTGTTCCCGCGCCTGGGGGCCCACCCCTGTCCA |
| | COSF1434 | CCGGACCTATAGTGGACCTGCTCCAG |
| | FGFR3- | GGACCTGGACCGTGTCCTTACCGTGACGTCCACCGACTTTAAGG |
| | TACC3.F17T8. | AGTCGGCCTTGAGGAAGCAGTCCTTATACCTCAAGTTCGACCCCC |
| | COSF1353 | TCCTGAGGGACAGTCCTGGTAGACC |
| | FGFR3- | GGACCTGGACCGTGTCCTTACCGTGACGTCCACCGACGTAAAGG |
| | TACC3.F17T11. | CGACACAGGAGGAGAACCGGGAGCTGAGGAGCAGGTGTGAGG |
| | COSF1348 | AGCTCCACGGGAAGAACCTGGAACTGGGGAAGATCATGGA |
| | FGFR3- | GGCCTTGTTTGACCGAGTCTACACTCACCAGAGTGACGTGTAAA |
| | TACC3.F15T11 | GGCGACACAGGAGGAGAACCGGGAGCTGAGGAGCAGGTGTGA |
| | | GGAGCTCCACGGGAAGAACCTGGAACTGGGGAAGATCATGGA |
| | FGFR3- | GGAGCTCTTCAAGCTGCTGAAGGAGGGCCACCGCATGGACAAG |
| | TACC3.F16T10. | CCCGCCAACTGCACACACGACCTGTGCCAGGCCCACCCCCAGGT |
| | COSF1359 | GTTCCCGCGCCTGGGGGCCCACCCCTGTCCACCGGACCTATAGT |
| | | GGACCTGCTCCAG |
| | FGFR3- | GGAGCTCTTCAAGCTGCTGAAGGAGGGCCACCGCATGGACAAG |
| | TACC3.F16T11. | CCCGCCAACTGCACACACGACCTGTAAAGGCGACACAGGAGGA |
| | COSF1348 | GAACCGGGAGCTGAGGAGCAGGTGTGAGGAGCTCCACGGGAA |
| | | GAACCTGGAACTGGGGAAGATCATGGA |
| | FGFR3- | GCACACACGACCTGTACATGATCATGCGGGAGTGCTGGCATGCC |
| | TACC3. | GCGCCCTCCCAGAGGCCCACCTTCAAGCAGAAGGAACTTTCCAA |
| | F17T13.NGS | AGCTGAAATCCAGAAAGTTCTAAAAGAAAAAGACCAACTTACCA |
| | | CAGATCTGAACTCCAT |
| | FGFR3- | GGACCTGGACCGTGTCCTTACCGTGACGTCCACCGACGCAGCTG |
| | TACC3.F17T5 | CATTCAGCCTCAGCGGAGGACACGCCTGTGGTGCAGTTGGCAGC |
| | | CGAGACCCCAACAGCAGAGAGCAAGGAGAGAGCCTT |
| | FGFR3- | GGACCTGGACCGTGTCCTTACCGTGACGTCCACCGACGAGAGAG |
| | TACC3.F17T6 | CCTTGAACTCTGCCAGCACCTCGCTTCCCACAAGCTGTCCAGGCA |
| | | GTGAGCCAGTGCCCACCCATCAGC |
| | FGFR3- | GGACCTGGACCGTGTCCTTACCGTGACGTCCACCGACCATGCAC |
| | TACC3.F17T9 | GGTGCAAATGAGACTCCCTCAGGACGTCCGCGGGAAGCCAAGCT |
| | | TGTGGAGTTCGATTTCTTGGGAGCACTGGACATTC |
| | FGFR3- | GGACCTGGACCGTGTCCTTACCGTGACGTCCACCGACGAGTACC |
| | TACC3.F18T7. | TGGAGCAGTTTGGAACTTCCTCGTTTAAGGAGTCGGCCTTGAGG |
| | NGS | AAGCAGTCCTTATACCTCAAGTTCGACCCCCTCCTGAGGGACAGT |
| | | CCTGGTAGACC |
| | FGFR3- | GTGACCGAGGACAACGTGATGAAGATCGCAGACTTCGGGCTGG |
| | TACC3.F14T11 | CCCGGGACGTGCACAACCTCGACGTAAAGGCGACACAGGAGGA |
| | | GAACCGGGAGCTGAGGAGCAGGTGTGAGGAGCTCCACGGGAA |
| | | GAACCTGGAACTGGGGAAGATCATGGA |
| | FGFR3- | GGCGCCTTTCGAGCAGTACTCCCCGAGCCAGCAGCTGCATTCAG |
| | TACC3. | CCTCAGCGGAGGACACGCCTGTGGTGCAGTTGGCAGCCGAGAC |
| | F18T4and5 | CCCAACAGCAGAGAGCAAGGAGAGAGCCTT |
| | FGFR3- | GGCGCCTTTCGAGCAGTACTCCCCGGGTGGCCAGGACACCCCCA |
| | TACC3. | GCTCCAGCTCCTCAGGGGACGACTCCGTGTTTGCCCACGACGTG |
| | F18T10.1 | CCAGGCCCACCCCCAGGTGTTCCCGCGCCTGGGGGCCCACCCCT |
| | | GTCCACCGGACCTATAGTGGACCTGCTCCAG |
| | FGFR3- | GGCGCCTTTCGAGCAGTACTCCCCGGGTGGCCAGGACACCCCCA |
| | TACC3. | GCTCCAGCTCCTCAGGGGACGAGGACCTGGATGCAGTGGTAAA |
| | F18T10 | GGCGACACAGGAGGAGAACCGGGAGCTGAGGAGCAGGTGTGA |
| | | GGAGCTCCACGGGAAGAACCTGGAACTGGGGAAGATCATGGA |
| | FGFR3- | GGCGCCTTTCGAGCAGTACTCCCCGGGTGTAAAGGCGACACAGG |
| | TACC3. | AGGAGAACCGGGAGCTGAGGAGCAGGTGTGAGGAGCTCCACG |
| | F18T11 | GGAAGAACCTGGAACTGGGGAAGATCATGGA |
| | FGFR3- | GGACCTGGACCGTGTCCTTACCGTGAATGGAATTCTACAGAAAC |
| | TACC3. | CAGTGGAGGCTGACACCGACCTCCTGGGGGATGCAAGCCCAGC |
| | TruncatedF17T4 | CTTTG |
| | FGFR3- | GCACACACGACCTGTACATGATCATGCGGGAGTGCTGGCATGCC |
| | TACC3.F17T7 | GCGCCCTCCCAGAGGCCCACCTTCAAGCAGCTGGTGGAGTACCT |
| | | GGAGCAGTTTGGAACTTCCTCGTTTAAGGAGTCGGCCTTGAGGA |
| | | AGCAGTCCTTATACCTCAAGTTCGACCCCCTCCTGAGGGACAGTC |
| | | CTGGTAGACC |
| | FGFR3- | GCACACACGACCTGTACATGATCATGCGGGAGTGCTGGCATGCC |
| | TACC3.F17T10 | GCGCCCTCCCAGAGGCCCACCTTCAAGCAGCTGGTGGAGGACCT |
| | | GGATGCAGTGGTAAAGGCGACACAGGAGGAGAACCGGGAGCT |
| | | GAGGAGCAGGTGTGAGGAGCTCCACGGGAAGAACCTGGAACTG |
| | | GGGAAGATCATGGA |
| | FGFR3- | GCACACACGACCTGTACATGATCATGCGGGAGTGCTGGCATGCC |
| | TACC3. | GCGCCCTCCCAGAGGCCCACCTTCAAGCAGCTGGTGGAGGACCT |
| | F17T11.1 | CCACGGGAAGAACCTGGAACTGGGGAAGATCATGGA |
| | FGFR3- | GCACACACGACCTGTACATGATCATGCGGGAGTGCTGGCATGCC |
| | TACC3. | GCGCCCTCCCAGAGGCCCACCTTCAAGCAGCAGGTGTGAGGAGC |
| | F17T11.2 | TCCACGGGAAGAACCTGGAACTGGGGAAGATCATGGA |
| | FGFR3- | GGTGCCACCCGCCTATGCCCCTCCCCCTGCCGTCCCCGGCCATCC |
| | TACC3. | TGCCCCCCAGAGTGCTGAGGTGTGGGGGGGGCCTTCTGGCCCAG |
| | F17intron | GTGCCCTGGCTGACCTGGACTGCTCAAGCTCTTCCCAGAGCCCAG |
| | 17T4.1 | GAA |
| | FGFR3- | GGTGCCACCCGCCTATGCCCCTCCCCCTGCCGTCCCCGGCCATCC |
| | TACC3. | CTCAGGACGTCCGCGGGAAGCCAAGCTTGTGGAGTTCGATTTCT |
| | F17Intron | TGGGAGCACTGGACATTC |
| | 17T9 | |
| | FGFR3- | GGACCTGGACCGTGTCCTTACCGTGACGTCCACCGACAACGAAG |
| | TACC3.F17T14 | AGTCACTGAAGAAGTGCGTGGAGGATTACCTGGCAAGGATCACC |
| | | CAGGAGGGCCAGAGGTACCAAGCCCTGAAGGCC |
| | FGFR3- | GGACCTGGACCGTGTCCTTACCGTGACGTCCACCGACGAGTACC |
| | TACC3. | TGGACCTGTCGGCGACACAGGAGGAGAACCGGGAGCTGAGGA |
| | F18T11del5 | GCAGGTGTGAGGAGCTCCACGGGAAGAACCTGGAACTGGGGAA |
| | | GATCATGGA |
| | FGFR3- | GGCGCCTTTCGAGCAGTACTCCCCGGGTGGCCAGGACACCCCCA |
| | TACC3.F18T1 | GCTCCAGCTCCTCAGGGGACGACTCCGGAGGTCCTGGGAGGGTC |
| | | AGTCTGGCCCGCCTGCCTGCTGACTTGGGTGTGGCCTGAGCAGG |
| | | TAAAGGCGACACAGGAGGAGAACCGGGAGCTGAGGAGCAGGT |
| | | GTGAGGAGCTCCACGGGAAGAACCTGGAACTGGGGAAGATCAT |
| | | GGA |
| | FGFR3- | GCACACACGACCTGTACATGATCATGCGGGAGTGCTGGCATGCC |
| | TACC3. | GCGCCCTCCCAGAGGCCCACCTTCAAGCAGCTGGTGGAAGGACC |
| | F17ins1T10 | TGGATGCAGTGGTAAAGGCGACACAGGAGGAGAACCGGGAGCT |
| | | GAGGAGCAGGTGTGAGGAGCTCCACGGGAAGAACCTGGAACTG |
| | | GGGAAGATCATGGA |
| | FGFR3- | GGACCTGGACCGTGTCCTTACCGTGACGTCCACCGACTGAAGGC |
| | TACC3. | CCACGCGGAGGAGAAGCTGCAGCTGGCAAACGAGGAGATCGCC |
| | F17T14.1 | CAGGTCCGGAGCAAGGCCCAGGCGGAAGCGTTGGCCCTCCAGG |
| | | CCAGCCTGAGGAAGGAGCAGA |
| | FGFR3- | GGTGCCACCCGCCTATGCCCCTCCCCCTGCCACGGAGGAGCCAG |
| | TACC3.F17T4 | GTCCCTGTCTGAGCCAGCAGCTGCATTCAGCCTCAGCGGAGGAC |
| | | ACGCCTGTGGTGCAGTTGGCAGCCGAGACCCCAACAGCAGAGA |
| | | GCAAGGAGAGAGCCTT |
| TERF2 | TERF2- | CCAAAGTACCCAAAGGCAAGTGGAACAGCTCTAATGGGGTTGAA |
| | JAK2.T8J19 | GAAAAGGAGACTTGGGTGGAAGAGGATGAACTGTTTCAAGTTC |
| | | AGGATTATGAACTATTAACAGAAAATGACATGTTACCAAATATGA |
| | | GGATAGGTGCCCTGGGGTTTTCTGGTGCCTTTGAAGACCGGGAT |
| | | C |
| TMEM106B | TMEM10 | AGATGGAAGAAATGGAGATGTCTCTCAGTTTCCATATGTGGAAT |
| | 6B- | TTACAGGAAGAGATAGTGTCACCTGCCCTACTTGTCAGGGAACA |
| | ROS1.T3R35 | GGAAGAATTCCTAGGGTCTGGCATAGAAGATTAAAGAATCAAAA |
| | | AAGTGCCAAGGAAGGGGTGACAGTGCTTATAAACGAAGACAAA |
| | | GAGTTGGCTGA |
| UBE2L3 | UBE2L3- | CAGGTCTGTCTGCCAGTAATTAGTGCCGAAAACTGGAAGCCAGC |
| | KRAS.U3K2. | AACCAAAACCGACCAAGGCCTGCTGAAAATGACTGAATATAAAC |
| | COSF1298.1 | TTGTGGTAGTTGGAGCTGGTGGCGTAGGCAAGAGTGCCTTGACG |
| | | ATACAGCTAATTCAG |
| USP10 | FGFR2- | CTCCCAGAGACCAACGTTCAAGCAGTTGGTAGAAGACTTGGATC |
| | USP10. | GAATTCTCACTCTCAAGTTGCTGGAGAATGTAACCCTAATCCATA |
| | F17del11U5 | AACCAGTGTCGTTGCAACCCCGTGGGCTGATCAATAAAGGGAAC |
| | | TGGTGCT |
| WRDR48 | WDR48- | CTGCAATTTGGGTTGCAACAACTAAGTCTACAGTAAATAAATGG |
| | PDGFRB.W9P12 | AAGCCACGTTACGAGATCCGATGGAAGGTGATTGAGTCTGTGAG |
| | | CTCTGACGGCCATGAGTACATCTACGTGGACCC |
| YAP1 | ESR1- | GCTTACTGACCAACCTGGCAGACAGGGAGCTGGTTCACATGATC |
| | YAP1.E6Y4 | AACTGGGCGAAGAGGGTGCCAGGTCCTCTTCCTGATGGATGGG |
| | | AACAAGCCATGACTCAGGATGGAGAAATTTACTATATAAACCAT |
| | | AAGAACAAGACCACCTCTT |
| ZEB2 | ZEB2- | CAGAGAGTGGCATGTATGCATGTGACTTATGTGACAAGACATTC |
| | PDGFRB.Z9P9 | CAGAAAAGCAGTTCCCTTCTGCGACATAAATACGAACACACAGTC |
| | | CCTGTCCGAGTGCTGGAGCTAAGTGAGAGCCACCCTGACAGTGG |
| | | GGAACAGACAGTC |
| ZMYND8 | ZMYND8- | GCTCGACCCTTGACCTTTCTGGCTCCAGAGAGACGCCCTCCTCCA |
| | RELA.Z21R2 | TTCTCTTAGGCTCCAACCAAGGCTCTGAACTGTTCCCCCTCATCTT |
| | | CCCGGCAGAGCCAGCCCAGGCCTCTGGCCCCTATGTGGAGATCA |
| | | TTGAGCAGC |

### Example 1

Detection of Genetic Biomarkers

1.1 Overview of Primer Design:

Primers for detecting each of the biomarkers listed in Table 2 were designed in accordance with conventional practice using techniques known to those skilled in the art. In general, primers of 18-30 nucleotides in length are optimal with a melting temperature (T m) between 65° C.-75° C. The GC content of the primers should be between 40-60%, with the 3′ of the primer ending in a C or G to promote binding. The formation of secondary structures within the primer itself is minimised by ensuring a balanced distribution of GC-rich and AT-rich domains. Intra/inter—primer homology should be avoided for optimal primer performance.

1.1.1 Primers for Copy Number Detection:

Primers were designed, as discussed in 1.1, to span the regions of the Table 2 genes as listed in Table 3. Several amplicons per gene were designed. Although the regions are given in Table 3 other regions within the genes in Table 2 could be used and a person skilled in the art would be able to identify the regions and design amplicons therefor. The depth of coverage is measured for each of these amplicons. The copy number amplification and deletion algorithm is based on a hidden Markov model (HMM). Prior to copy number determination, read coverage is corrected for GC bias and compared to a preconfigured baseline.

1.1.2 Primer for Hotspot Detection:

Primers were designed, as discussed in 1.1, to target specific regions prone to oncogenic somatic mutations as listed Table 3 and in consideration with the general points discussed above.

1.1.3 Primers for Fusion Detection:

Primers were designed, as discussed in 1.1, to target specific regions prone to gene rearrangement as listed Table 3 and in consideration with the general points discussed above.

1.1.4 Primers for Quantitative Detection of PD-L1 mRNA by NGS:

Extracted RNA is processed via RT-PCR to create complementary DNA (cDNA) which is then amplified using primers designed, as discussed in 1.1. Multiple primer sets were designed to span the exon/intron boundaries across the PD-L1 gene and are listed in Table 4 in FIG. 6.

1.2 DNA and RNA Extraction

DNA and RNA were extracted from a formalin fixed tumour sample. Two xylene washes were performed by mixing 1 ml of xylene with the sample. The samples were centrifuged and xylene removed. This was followed by 2 washes with 1 ml of pure ethyl alcohol. After the samples were air-dried, 25 μl of digestion buffer, 75 μl of nuclease free water and 4 μl of protease were added to each sample. Samples were then digested at 55° C. for 3 hours followed by 1 hour digestion at 90° C.

120 μl of Isolation additive was mixed with each sample and the samples added to filter cartridges in collection tubes and centrifuged. The filters were moved to new collection tubes and kept in the fridge for DNA extraction at a later stage. The flow-through was kept for RNA extraction and 275 μl of pure ethyl alcohol was added and the sample moved to a new filter in a collection tube and centrifuged. After a wash of 700 μl of Wash 1 buffer the RNA was treated with DNase as follows; a DNase mastermix was prepared using 6 μl of 10× DNase buffer, 50 μl of nuclease free water and 4 μl of DNase per sample. This was added to the centre of each filter and incubated at room temperature for 30 minutes.

After the incubation 3 washes were performed using Wash 1, then Wash 2/3 removing the wash buffer from the collection tubes after each centrifugation. The filters were moved to a new collection tube and the elution solution (heated to 95° C.) was added to each filter and incubated for 1 minute. After centrifuging the sample, the filter was discarded and the RNA collected in the flow through moved to a new low bind tube.

The DNA in the filters were washed with Wash 1 buffer, centrifuged and flow through discarded. The DNA was treated with RNase (50 μl nuclease water and 10 μl RNase) and incubated at room temperature for 30 minutes. As above with the RNA, three washes were completed and the samples eluted in elution solution heated at 95° C.

1.3 DNA and RNA Measurement

The quantity of DNA and RNA from the extracted samples were measured using the Qubit® 3.0 fluorometer and the Qubit® RNA High Sensitivity Assay kit (CAT: Q32855) and Qubit® dsDNA High Sensitivity Assay kit (Cat: Q32854). 1 μl of RNA/DNA combined with 199 μl of combined HS buffer and reagent were used in Qubit® assay tubes for measurement. 10111 of standard 1 or 2 were combined with 190 μl of the buffer and reagent solution for the controls.

1.4 Library Preparation

RNA samples were diluted to 5 ng/μl if necessary and reverse transcribed to cDNA in a 96 well plate using the SuperScript VILO cDNA synthesis kit (CAT 11754250). A mastermix of 2 μl of VILO, 1 μl of 10× SuperScript III Enzyme mix and 5 μl of nuclease free water was made for all of the samples. 8 μl of the MasterMix was used along with 2 μl of the RNA in each well of a 96 well plate. The following program was run:

| Temperature | Time |
|---|---|
| 42° C. | 30 min |
| 85° C. | 5 min |
| 10° C. | Hold |

Amplification of the cDNA was then performed using 4 μl of 6 RNA primers covering multiple exon-intron loci across the gene, 4 μl of AmpliSeq HiFi*¹ and 2 μl of nuclease free water into each sample well. The plate was run on the thermal cycler for 30 cycles using the following program:

| Stage | Step | Temperature | Time | |
|---|---|---|---|---|
| Hold | Activate the enzyme | 99° C. | 2 | min |
| Cycle | Denature | 99° C. | 15 | sec |
| (30 cycles) | Anneal and extend | 60° C. | 4 | min |

| | | | |
|---|---|---|---|
| Hold | — | 10° C. | Hold |

DNA samples were diluted to 5 ng/μl and added to AmpliSeq Hifi*¹, nuclease free water and set up using two DNA primer pools (5 μl of pool 1 and 5 μl of pool 2) in a 96 well plate. The following program was run on the thermal cycler:

| Stage | Step | Temperature | Time | |
|---|---|---|---|---|
| Hold | Activate the enzyme | 99° C. | 2 | min |
| Cycle | Denature | 99° C. | 15 | sec |
| (18 cycles) | Anneal and extend | 60° C. | 14 | min |

| | | | |
|---|---|---|---|
| Hold | — | 10° C. | Hold (up to |
| | | | 16 hours) |

Following amplification, the amplicons were partially digested using 2 μl of LIB Fupa*¹, mixed well and placed on the thermal cycler on the following program:

| Temperature | Time |
|---|---|
| 50° C. | 10 min |
| 55° C. | 10 min |
| 60° C. | 20 min |
| 10° C. | Hold (for up |
| | to 1 hour) |

4 μl of switch solution*¹, 2 μl of diluted Ion XPRESS Barcodes 1-16 (CAT: 4471250) and 2 μl of LIB DNA ligase*¹ were added to each sample, mixing thoroughly in between addition of each component. The following program was run on the thermal cycler:

| Temperature | Time |
|---|---|
| 22° C. | 30 min |
| 72° C. | 10 min |
| 10° C. | Hold (for up |
| | to 1 hour) |

The libraries were then purified using 30 μl of Agencourt AMPure XP (Biomeck Coulter cat: A63881) and incubated for 5 minutes. Using a plate magnet, 2 washes using 70% ethanol were performed. The samples were then eluted in 50 μl TE.

1.5 qPCR

The quantity of library was measured using the Ion Library TaqMan quantitation kit (cat: 4468802). Four 10-fold serial dilutions of the E. coli DH10B Ion control library were used (6.8 pmol, 0.68 pmol, 0.068 pmol and 0.0068 pmol) to create the standard curve. Each sample was diluted 1/2000, and each sample, standard and negative control were tested in duplicate. 10 μl of the 2× TaqMan mastermix and 1 μl of the 20× TaqMan assay were combined in a well of a 96 well fast thermal cycling plate for each sample. 9 μl of the 1/2000 diluted sample, standard or nuclease free water (negative control) were added to the plate and the qPCR was run on the ABI StepOnePlus™ machine (Cat: 4376600) using the following program:

| Stage | Temperature | Time | |
|---|---|---|---|
| Hold (UDG incubation) | 50° C. | 2 | min |
| Hold (polymerase activation) | 95° C. | 20 | sec |
| Cycle (40 cycles) | 95° C. | 1 | sec |
| | 60° C. | 20 | sec |

Samples were diluted to 100 pmol using TE and 10 μl of each sample pooled to either a DNA tube or RNA tube. To combine the DNA and RNA samples, a ratio of 80:20 DNA:RNA was used.

1.6 Template Preparation

The Ion One Touch™ 2 was initialized using the Ion S5 OT2 solutions and supplies*² and 150 μl of breaking solution*² was added to each recovery tube. The pooled RNA samples were diluted further in nuclease free water (8 μl of pooled sample with 92 μl of water) and an amplification mastermix was made using the Ion S5 reagent mix*² along with nuclease free water, ION S5 enzyme mix*², Ion sphere particles (ISPs)*² and the diluted library. The mastermix was loaded into the adapter along with the reaction oil*². The instrument was loaded with the amplification plate, recovery tubes, router and amplification adapter loaded with sample and amplification mastermix.

1.7 Enrichment

For the enrichment process, melt off was made using 280 μl of Tween*² and 40 μl of 1M Sodium Hydroxide. Dynabeads® MyOne™ Streptavidin C1 (CAT: 65001) were washed with the OneTouch wash solution*² using a magnet. The beads were suspended in 130 μl of MyOne bead capture solution*². The ISPs were recovered by removing the supernatant, transferring to a new low bind tube and subsequently washed in 800 μl of nuclease free water. After centrifuging the sample and removing the supernatant of water, 20 μl of template positive ISPs remained. 80 μl of ISP resuspension solution*² was added for a final volume of 100 μl.

A new tip, 0.2 ml tube and an 8 well strip was loaded on the OneTouch™ ES machine with the following:
Well 1: 100 μl of template positive ISPs
Well 2: 130 μl of washed Dynabeads® MyOne™ streptavidin C1 beads, resuspended in MyOne bead capture
Well 3: 300 μl of Ion OneTouch ES Wash solution*²
Well 4: 300 μl of Ion OneTouch ES Wash solution
Well 5: 300 μl of Ion OneTouch ES Wash solution
Well 6: Empty
Well 7: 300 μl of melt off
Well 8: Empty

Following the run which takes approximately 35 minutes, the enriched ISPs were centrifuged, the supernatant removed and washed with 200 μl of nuclease free water. Following a further centrifuge step and supernatant removal, 10 μl of ISPs remained. 90 μl of nuclease free water was added and the beads were resuspended.

1.8 Sequencing

The Ion S5 System™ (Cat: A27212) was Initialized Using the Ion S5 Reagent Cartridge, Ion S5 cleaning solution and Ion S5 wash solutions*².

5 μl of Control ISPs*² were added to the enriched sample and mixed well. The tube was centrifuged and the supernatant removed to leave the sample and control ISPs. 15 μl of Ion S5 annealing buffer*² and 20 μl of sequencing primer*² were added to the sample. The sample was loaded on the thermal cycler for primer annealing at 95° C. for 2 minutes and 37° C. for 2 minutes. Following thermal cycling, 10 μl of Ion S5 loading buffer*² was added and the sample mixed.

50% annealing buffer was made using 500 μl of Ion S5 annealing buffer*² and 500 μl of nuclease free water*².

The entire sample was then loaded into the loading port of an Ion 540™ chip (Cat: A27766) and centrifuged in a chip centrifuge for 10 minutes.

Following this, 100 μl of foam (made using 49 μl of 50% annealing buffer and 1 μl of foaming solution*²) was injected into the port followed by 55 μl of 50% annealing buffer into the chip well, removing the excess liquid from the exit well. The chip was centrifuged for 30 seconds with the chip notch facing out. This foaming step was repeated.

The chip was flushed twice using 100 μl of flushing solution (made using 250 μl of isopropanol and 250 μl of Ion S5 annealing buffer) into the loading port, and excess liquid removed from the exit well. 3 flushes with 50% annealing buffer into the loading port were then performed. 60 μl of 50% annealing buffer was combined with 6 μl of Ion S5 sequencing polymerase*². 65 μl of the polymerase mix was then loaded into the port, incubated for 5 minutes and loaded on the S5 instrument for sequencing which takes approximately 3 hours and 16 hours for data transfer.
*1 From the Ion Ampliseq™ library 2.0 (Cat: 4480441)
*2 From the Ion 540™ OT2 kit (Cat: A27753)

1.9 Data Analysis

1.9.1 DNA Cnv Analysis:

Copy number variations (CNVs) represent a class of variation in which segments of the genome have been duplicated (gains) or deleted (losses). Large, genomic copy number imbalances can range from sub-chromosomal regions to entire chromosomes.

Raw data were processed on the Ion S5 System and transferred to the Torrent Server for primary data analysis. The Baseline v2.0 plug-in is included in Torrent Suite Software, which comes with each Ion Torrent™ sequencer. Copy number amplification and deletion detection was performed using an algorithm based on a hidden Markov model (HMM). The algorithm uses read coverage across the genome to predict the copy-number.

Prior to copy number determination, read coverage is corrected for GC bias and compared to a preconfigured baseline.

The median of the absolute values of all pairwise differences (MAPD) score is reported per sample and is used to assess sample variability and define whether the data are useful for copy number analysis. MAPD is a per-sequencing run estimate of copy number variability, like standard deviation (SD). If one assumes the log 2 ratios are distributed normally with mean 0 against a reference a constant SD, then MAPD/0.67 is equal to SD. However, unlike SD, using MAPD is robust against high biological variability in log 2 ratios induced by known conditions such as cancer. Samples with an MAPD score above 0.5 should be carefully reviewed before validating CNV call.

The results from copy number analysis after normalisation can be visualised from the raw data.

Somatic CNV detection provides Confidence bounds for each Copy Number Segment. The Confidence is the estimated percent probability that Copy Number is less than the given Copy Number bound. A lower and upper percent and the respective Copy Number value bound are given for each CNV. Confidence intervals for each CNV are also stated, and amplifications of a copy number>6 with the 5% confidence value of ≥4 after normalization and deletions with 95% CI≤1 are classified as present.

DNA Hotspot Analysis:

Raw data were processed on the Ion S5 System and transferred to the Torrent Server for primary data analysis performed using the custom workflow. Mapping and alignment of the raw data to a reference genome is performed and then hotspot variants are annotated in accordance with the BED file. Coverage statistics and other related QC criteria are defined in a vcf file which includes annotation using a rich set of public sources. Filtering parameters can be applied to identify those variants passing QC thresholds and these variants can be visualised on IGV. In general, the rule of classifying variants with >10% alternate allele reads, and in >10 unique reads are classified as ‘detected’. Several in-silico tools are utilised to assess the pathogenicity of identified variants these include PhyloP, SIFT, Grantham, COSMIC and PolyPhen-2.

1.9.2 RNA Expression Analysis:

RNA Expression Analysis:

The custom bioinformatics workflow extracts sequencing data from the Ion Torrent server, this pipeline executes global normalisation, followed by the removal of libraries with <25,000 reads. The resulting data is normalised per million and the linear scale converted to a log scale transforming zeros to 0.5. stable control amplicons included in the panel design allow for further robust data normalisation. The pipeline includes a size factor calculation comparing the median difference for every sample compared to controls. The size factor is subtracted from all measurements in the original sequence data. The end point of this bioinformatics pipeline is a CSV file containing log 2 RPM per amplicon.

The bespoke BED file is a formatted to contain the nucleotide positions of each amplicon per transcript in the mapping reference. Reads aligning to the expected amplicon locations and meeting filtering criteria such as minimum alignment length are reported as percent “valid” reads. “Targets Detected” is defined as the number of amplicons detected (≥10 read counts) as a percentage of the total number of targets.

After mapping, alignment and normalization, the AnnpliSeqRNA plug-in provides data on QC metrics, visualization plots, and normalized counts per gene that corresponds to gene expression information that includes a link to a downloadable file detailing the read counts per gene in a tab delimited text file. The number of reads aligning to a given gene target represents an expression value referred to as “counts”. This Additional plug-in analyses include output for each barcode of the number of genes (amplicons) with at least 1, 10, 100, 1,000, and 10,000 counts to enable determination of the dynamic range and sensitivity per sample.

A summary table of the above information, including mapping statistics per barcode of total mapped reads, percentage on target, and percentage of panel genes detected (“Targets Detected”) is viewable in Torrent Suite Software to quickly evaluate run and library performance.

1.9.3 Fusion Analysis:

Raw data were processed on the Ion S5 System and transferred to the Torrent Server for primary data analysis performed using the custom workflow. For each sample the following 6 internal expression quality controls are also monitored: HMBS, ITGB7, MYC, LRP1, MRPL13 and TBP. The expression controls are spiked into each sample and confirm the assay is performing as expected for RNA analysis. The controls must be present with at least 15 reads.

The BED used contains details of the fusion break points and allows for accurate mapping of known fusion genes. The software automatically assesses each targeted fusion to check 70% of the Insert is covered by the read on both sides of the breakpoint. Within that 70% overlap, at least 66.66% exact matches are required. The software automatically fails for regions not meeting this criteria. The read counts for each targeted fusion event which passes the initial QC metrics is recorded and visible in the raw data. Targeted gene fusions (except EGFR VIII and MET exon 14 del) are reported when detected with >40 read counts and meeting the thresholds of assay specific internal RNA quality control with a sensitivity>99% and PPV of >99%.

In addition to these targeted events it is also possible to detect non-targeted fusions, which occur when the primers for a targeted fusion bind to and produce a product of two genes which are targeted but not in that particular configuration. Non-targeted gene fusions (including EGFR VIII and MET exon 14 del) are reported when detected with >1000 read counts and meeting the thresholds of assay specific internal RNA quality control with a sensitivity of >99% and PPV of >99%.

1.9.4 TMB Analysis

Raw data were processed on the Ion S5 System and transferred to the Torrent Server for data analysis performed using the Oncomine Tumor Mutation Load—w2.0—DNA—Single Sample workflow. To meet QC acceptance the sample must have an average coverage/mean depth of >300, uniformity of >80% and a deamination score of <30.

The following calculation is applied to sample which pass to QC to calculate the TMB figure:

Non-synonymous somatic mutations×10⁶/total exonic bases with sufficient coverage
1. If the pre-calibration figure is >25 then

Mutation load=(Pre calibration mutation load−25)×calibration slope+25
2. If the pre calibration figure is <25 then no calibration required.

### Example 2

Analysis of Tumour Mutational Burden.

2.0 DNA Measurement

DNA from a FFPE tumour sample was quantified post extraction following the protocol in section 1.3 above.

2.1 Library Preparation

DNA samples were diluted to 5 ng/μl and added to 5× Ion AmpliSeq Hifi (from the Ion AmpliSeq™ library kit plus (4488990)), nuclease free water and set up using two DNA primer pools (5 μl of pool 1 and 5 μl of pool 2) in a 96 well plate. The list of genes targeted for TMB analysis is shown in Table 5. The following program was run on the thermal cycler:

| Stage | Step | Temperature | Time | |
|---|---|---|---|---|
| Hold | Activate the enzyme | 99° C. | 2 | min |
| Cycle | Denature | 99° C. | 15 | sec |
| (15) | Anneal and extend | 60° C. | 16 | min |

| | | | |
|---|---|---|---|
| Hold | — | 10° C. | Hold |

Following amplification, the amplicons were partially digested using 2 μl of LIB FuPa (From the Ion 540™ OT2 kit (Cat: A27753)), mixed well and placed on the thermal cycler on the following program:

| Temperature | Time |
|---|---|
| 50° C. | 20 min |
| 55° C. | 20 min |
| 60° C. | 20 min |
| 10° C. | Hold (for up |
| | to 1 hour) |

4 μl of switch solution*³, 2 μl of diluted Ion XPRESS Barcodes 1-16 (Cat: 4471250) and 2 μl of LIB DNA ligase (From the Ion Ampliseq™ library kit plus (4488990)) were added to each sample, mixing thoroughly in between addition of each component. The following program was run on the thermal cycler:

| Temperature | Time |
|---|---|
| 22° C. | 30 min |
| 68° C. | 5 min |
| 72° C. | 5 min |
| 10° C. | Hold (for up |
| | to 24 hour) |

2.2 Purification

Libraries were purified as in section 1.3 using 45 μl of Agencourt AMPure XP (Biomeck Coulter cat: A63881).

2.3 q-PCR

The quantity of library was measured using the Ion Library TaqMan quantitation kit (cat: 4468802). Three 10-fold serial dilutions of the E. coli DH10B Ion control library were used (6.8 pmol, 0.68 pmol and 0.068 pmol) to create the standard curve. Each sample was diluted 1/500 and each sample, standard and negative control were tested in duplicate. 10 μl of the 2× TaqMan mastermix and 1 μl of the 20× TaqMan assay were combined in a well of a 96 well fast thermal cycling plate for each sample. 9 μl of the 1/500 diluted sample, standard or nuclease free water (negative control) were added to the plate and the qPCR was run on the ABI StepOnePlus™ machine (Cat: 4376600) using the program listed in section 1.5.

Samples were diluted to 100 pMol using the results from the q-PCR and pooled ready for template preparation. Following this, template preparation, enrichment of the sample and sequencing were performed as written in sections 1.6, 1.7 and 1.8, respectively.

TABLE 5
Genes targeted for analysis of Tumour Mutational Burden (TMB).

| | | | | | | | |
|---|---|---|---|---|---|---|---|
| ABL1 | CCNE1 | EPHB4 | GPR124 | MAF | NFKB2 | PPARG | SSX1 |
| ABL2 | CD79A | EPHB6 | GRM8 | MAFB | NIN | PPP2R1A | STK11 |
| ACVR2A | CD79B | ERBB2 | GUCY1A2 | MAGEA1 | NKX2-1 | PRDM1 | STK36 |
| ADAMTS20 | CDC73 | ERBB3 | HCAR1 | MAGI1 | NLRP1 | PRKAR1A | SUFU |
| AFF1 | CDH1 | ERBB4 | HIF1A | MALT1 | NOTCH1 | PRKDC | SYK |
| AFF3 | CDH11 | ERCC1 | HLF | MAML2 | NOTCH2 | PSIP1 | SYNE1 |
| AKAP9 | CDH2 | ERCC2 | HNF1A | MAP2K1 | NOTCH4 | PTCH1 | TAF1 |
| AKT1 | CDH20 | ERCC3 | HOOK3 | MAP2K2 | NPM1 | PTEN | TAF1L |
| AKT2 | CDH5 | ERCC4 | HRAS | MAP2K4 | NRAS | PTGS2 | TAL1 |
| AKT3 | CDK12 | ERCC5 | HSP90AA1 | MAP3K7 | NSD1 | PTPN11 | TBX22 |
| ALK | CDK4 | ERG | HSP90AB1 | MAPK1 | NTRK1 | PTPRD | TCF12 |
| APC | CDK6 | ESR1 | ICK | MAPK8 | NTRK3 | PTPRT | TCF3 |
| AR | CDK8 | ETS1 | IDH1 | MARK1 | NUMA1 | RAD50 | TCF7L1 |
| ARID1A | CDKN2A | ETV1 | IDH2 | MARK4 | NUP214 | RAF1 | TCF7L2 |
| ARID2 | CDKN2B | ETV4 | IGF1R | MBD1 | NUP98 | RALGDS | TCL1A |
| ARNT | CDKN2C | EXT1 | IGF2 | MCL1 | PAK3 | RARA | TET1 |
| ASXL1 | CEBPA | EXT2 | IGF2R | MDM2 | PALB2 | RB1 | TET2 |
| ATF1 | CHEK1 | EZH2 | IKBKB | MDM4 | PARP1 | RECQL4 | TFE3 |
| ATM | CHEK2 | FAM123B | IKBKE | MEN1 | PAX3 | REL | TGFBR2 |
| ATR | CIC | FANCA | IKZF1 | MET | PAX5 | RET | TGM7 |
| ATRX | CKS1B | FANCC | IL2 | MITF | PAX7 | RHOH | THBS1 |
| AURKA | CMPK1 | FANCD2 | IL21R | MLH1 | PAX8 | RNASEL | TIMP3 |
| AURKB | COL1A1 | FANCF | IL6ST | MLL | PBRM1 | RNF2 | TLR4 |
| AURKC | CRBN | FANCG | IL7R | MLL2 | PBX1 | RNF213 | TLX1 |
| AXL | CREB1 | FANCJ | ING4 | MLL3 | PDE4DIP | ROS1 | TNFAIP3 |
| BAI3 | CREBBP | FAS | IRF4 | MLLT10 | PDGFB | RPS6KA2 | TNFRSF14 |
| BAP1 | CRKL | FBXW7 | IRS2 | MMP2 | PDGFRA | RRM1 | TNK2 |
| BCL10 | CRTC1 | FGFR1 | ITGA10 | MN1 | PDGFRB | RUNX1 | TOP1 |
| BCL11A | CSF1R | FGFR2 | ITGA9 | MPL | PER1 | RUNX1T1 | TP53 |
| BCL11B | CSMD3 | FGFR3 | ITGB2 | MRE11A | PGAP3 | SAMD9 | TPR |
| BCL2 | CTNNA1 | FGFR4 | ITGB3 | MSH2 | PHOX2B | SBDS | TRIM24 |
| BCL2L1 | CTNNB1 | FH | JAK1 | MSH6 | PIK3C2B | SDHA | TRIM33 |
| BCL2L2 | CYLD | FLCN | JAK2 | MTOR | PIK3CA | SDHB | TRIP11 |
| BCL3 | CYP2C19 | FLI1 | JAK3 | MTR | PIK3CB | SDHC | TRRAP |
| BCL6 | CYP2D6 | FLT1 | JUN | MTRR | PIK3CD | SDHD | TSC1 |
| BCL9 | DAXX | FLT3 | KAT6A | MUC1 | PIK3CG | Sep-09 | TSC2 |
| BCR | DCC | FLT4 | KAT6B | MUTYH | PIK3R1 | SETD2 | TSHR |
| BIRC2 | DDB2 | FN1 | KDM5C | MYB | PIK3R2 | SF3B1 | UBR5 |
| BIRC3 | DDIT3 | FOXL2 | KDM6A | MYC | PIM1 | SGK1 | UGT1A1 |
| BIRC5 | DDR2 | FOXO1 | KDR | MYCL1 | PKHD1 | SH2D1A | USP9X |
| BLM | DEK | FOXO3 | KEAP1 | MYCN | PLAG1 | SMAD2 | VHL |
| BLNK | DICER1 | FOXP1 | KIT | MYD88 | PLCG1 | SMAD4 | WAS |
| BMPR1A | DNMT3A | FOXP4 | KLF6 | MYH11 | PLEKHG5 | SMARCA4 | WHSC1 |
| BRAF | DPYD | FZR1 | KRAS | MYH9 | PML | SMARCB1 | WRN |
| BRD3 | DST | G6PD | LAMP1 | NBN | PMS1 | SMO | WT1 |
| BTK | EGFR | GATA1 | LCK | NCOA1 | PMS2 | SMUG1 | XPA |
| BUB1B | EML4 | GATA2 | LIFR | NCOA2 | POT1 | SOCS1 | XPC |
| CARD11 | EP300 | GATA3 | LPHN3 | NCOA4 | POU5F1 | SOX11 | XPO1 |
| CASC5 | EP400 | GDNF | LPP | NF1 | | SOX2 | XRCC2 |
| CBL | EPHA3 | GNA11 | LRP1B | NF2 | | SRC | ZNF384 |
| CCND1 | EPHA7 | GNAQ | LTF | NFE2L2 | | | ZNF521 |
| CCND2 | EPHB1 | GNAS | LTK | NFKB1 | | | |

### Example 3

Immunofocus® IHC Assay

The Immunofocus assay was validated for clinical use and accredited by CLIA and by UKAS (9376) in compliance with IS015189:2012. PD-L1 rabbit monoclonal antibody (clone E1L3N) was obtained from Cell Signalling (Cat No. 136845). Histological sections from a representative PWET block for each case were cut at 3 μm thickness and mounted on Super Frost glass slides (Leica, cat no). Section deparaffinization, antigen retrieval and immunohistochemical labelling were performed using the Bond III Autostainer and Bond Polymer Refine Detection Kit (Leica, Cat no. DS8900) according to the manufacturer's instructions. Primary antibody was applied for 20 minutes at 1/400 dilution. Assessment of PD-L1 immunostaining was performed by a qualified histopathologist in accordance with PD-L1 clinical reporting guidelines.

Results

PD-L1 IHC Expression Analysis

Using a cut point of a 10% tumour proportion score, elevated levels of PD-L1 expression were identified in 19.5% of cases as shown in Table 6. This information is presented in a pie chart format in FIG. 2.

TABLE 6

| PD-L1 Tumour | | % of samples |
|---|---|---|
| Proportion Score | Frequency | (n = 1099) |
| <1 | 671 | 61.1% |
| 1-10 | 214 | 19.5% |
| 11-25 | 67 | 6.1% |
| 26-50 | 44 | 4.0% |
| 50+ | 103 | 9.4% |
| Total | 1099 | 100% |
| 0-10 | 885 | 80.5% |
| 11+ | 214 | 19.5% |

DDR Gene Genomic Analysis of Variants

As shown in Table 7, DDR genomic variants were identified in 130 cases with PD-L1 expression levels with a tumour proportion score (TPS)>10%. Thirty of the 95 DDR genes (32%) analysed harboured genetic variants in conjunction with elevated (TPS>10%) PD-L1 expression levels. The DDR aberrant genes associated with high expression levels of PD-L1 comprises AKT1, TP53, ATM, BRCA2, FANCD2, MLH1, PTEN, NBN, PMS2, ATR, AKT2, MSH6, RB1, BRCA1, IDH1, IDH2, ARID1A, CHEK2, BAP1, CREBBP, SETD2, SLX4, RNF43, NF1, GNAS, NF2, NOTCH1, DDR2 and AXL.

TABLE 7

| | No DDR | DDR | | |
|---|---|---|---|---|
| | variant | variant | | |
| | detected | detected | | |
| <1 | 212 | 459 | % unique samples with DDR genes | 67.5% |
| 1-10 | 61 | 155 | % of samples with PD-L1 | 19.5% |
| | | | score greater than 10% | |
| 11-25 | 29 | 38 | % of samples with DDR genes or | 75.2% |
| | | | PD-L1 score >10% | |
| 26-50 | 20 | 24 | % of DDR samples with PD-L1 | 11.8% |
| | | | above 10% | |
| >50 | 36 | 68 | | |
| Total | 358 | 744 | | |

Pd-L1 Ngs mRNA Analysis:

FIG. 3 shows analytical validation of the quantitative measurements of mRNA levels by NGS in FFPE samples, consisting of PD-L1 expressing control cell lines, using PD-L1 expression as an example. PD-L1 mRNA expression levels are measured using next generation sequencing (NGS) analysis to provide a readout measured in RPM (Reads per million mapped reads). The RPM reads were first normalised and a log score generated to derive a nLRPM. The nLRPM counts are used as a surrogate measure of mRNA gene expression. Four cell line controls stably expressing variable levels of PD-L1 assessed by PD-L1 protein were selected representing tumour proportions score of 0%, 25%, 75% and 100% as assessed at the protein level by immunocytochemistry. The nLRPM counts are shown for two primer sets spanning exon/intron boundaries for the PD-L1 gene.

A) shows nLRPM counts from the two different amplicons targeting the PD-L1 gene.

B) shows PD-L1 nLRPM counts (mRNA) generated by the method of the present invention compared to PD-L1 protein expression assessed by IHC.

C) shows photomicrographs of four cell line controls immunohistochemically stained with an antibody against PD-L1 and expressing different levels of PD-L1 protein together with the observed tumour proportion score (TPS).

FIG. 4 shows a correlation of PD-L1 expression by IHC with PD-L1 mRNA expression by NGS as non-normalised RPM counts in nine formalin fixed, paraffin embedded samples of non small cell lung cancer (NSCLC)

A) shows RPM counts from the two different amplicons targeting the PD-L1 gene

B) shows PD-L1 RPM counts (mRNA) generated by the method of the present invention compared to PD-L1 protein expression assessed by IHC.

C) shows photomicrographs of a representative sample of NSCLC stained with hematoxylin and eosin and immunohistochemically stained with an antibody against PD-L1.

The data shows that the method of the present invention provides an accurate quantitative assessment of mRNA expression when applied to routine formalin fixed paraffin wax embedded samples. Notably the RPM shows a rapid increase in parallel with protein expression as measured by IHC across cut point values of 1%, 10%, 25% and 50%. These are the clinically important cut points defined by a number of approved IHC Cdx PD-L1 assays for the identification of responders to anti-PD-L1/PD-1 directed 10 immunotherapies (eg VENTANA PD-L1 (SP263) Assay, VENTANA PD-L1 (SP142) Assay, Dako PD-L1).

Validation of Normalised Log of Reads Per Million (nLRPM) and Establishment of Cut-Offs

FIG. 5 shows normalised log reads per million (nLRPM) plotted against combined PD-L1 score [Combined PD-L1 expression score=tumour content×PD-L1 positive tumour cells+PIC score× PD-L1 positive ICs]. RPM counts were normalised against expression of housekeeping genes and chip coverage to account for run inter-variability. PD-L1 mRNA expression measured by NGS (nLRPM) was plotted against PD-L1 IHC combined PD-L1 expression score. PD-L1 expression combined score cut-offs of clinical relevance were established as follows: negative (<1%): <6 nLRPM; 1-10%: 6.1-7.1 nLRPM; 10-25%: 7.2-8.5 nLRPM; 25-50%: 8.6-10 nLRPM: >50%: >10 nLRPM.

Analysis of Tumour Mutational Burden.

Analysis of TMB was performed on 44 solid tumour samples. Fifteen cases were associated with DDR mutations and 29 cases showed aberration of DDR genes. Notably no significant difference was observed in tumour mutation burden (TMB) between the two groups (Table 8). This shows that TMB and DDR defects are two entirely independent mechanisms that can predict response to agents targeting the immune-checkpoint including components of the PD1/PD-L1 pathway, or alternatively agents targeting DDR signalling pathway including PARP inhibitors, DDR inhibitors (e.g. ATR) and cell cycle checkpoint inhibitors (e.g. Cdc7 inhibitors), or a combination of immune-checkpoint inhibitors and DDR inhibitors and that both these variables need to be assessed to accurately determine response to the above therapies or other therapeutic agents targeting the immune-checkpoint pathways

TABLE 8
Correlation of TMB and DDR status

| | DDR variant detected | No DDR variant detected |
|---|---|---|
| | (n = 15) | (n = 29) |
| Average TMB | 5.43 | 8.45 |
| Mode TMB | 0.84 | 2.51 |
| Median TMB | 4.18 | 4.99 |

PD-L1-DDR-TMB Immune Signature Algorithm

In the present invention, we have shown that a proportion of solid tumours are characterised not only by high PD-L1 mRNA and protein expression levels but also aberration of a specific set of DDR genes. Aberration of DDR genes results in genomic instability which results in increased expression of neoantigens which enhances the immune response against the tumour.

The quantitative assessment of NGS PD-L1 mRNA expression using nLRPM as a readout provides a more accurate assessment of PD-L1 immune status than microscopic scoring of PD-L1 IHC staining by a pathologist. This approach circumvents the problem of inter-observer variability associated with the reading of IHC immunostains by the pathologist and enables the analysis of immune-checkpoint and DDR biomarkers to be integrated into a combinatorial algorithm.

This molecular signature combining these elements can, therefore, help identify those patients most likely to respond to an agent, for example, targeting the immune-checkpoint including components of the PD1/PD-L1 pathway, or alternatively agents targeting DDR signalling pathway including PARP inhibitors, DDR inhibitors (e.g. ATR) and cell cycle checkpoint inhibitors (e.g. Cdc7 inhibitors), or a combination of immune-checkpoint inhibitors and DDR inhibitors and thereby circumvent the problems associated with the current goldstandard PD-L1 IHC assays [Ventana PD-L1 (SP263 & SP142), Dako PD-L1 IHC (28-8 & 22C3)].

The NGS signature platform enables all biomarkers of response to be run in a high throughput testing configuration in which PD-L1 expression can be integrated with genomic aberrations in DDR genes and TMB.

### Example 4

Application of Polygenic Prediction Score (PPS) Algorithm to Results

Case 1. Results obtained from a tumour biopsy sample of a patient with Non-small Cell Lung Cancer. Assay results:
A. Tumour Type: Non-Small Cell Lung Cancer
B. PD-L1 nLRPM=2.2 (PPS=1)
C. DDR Status=BRCA1, SETD2 SNV hotspot mutations (PPS=2)
D. TMB=13 mut/MB DNA (PPS=1)

PPS Algorithm score=4

Indicative of moderate response to immunotherapy and DDR inhibitors

1. A method for determining the susceptibility of a patient suffering from proliferative disease to treatment using an agent targeting a cell pathway or components thereof comprising an immune-checkpoint comprising components of the PD1/PD-L1 pathway, an agent targeting DDR signalling pathway comprising PARP inhibitors, DDR inhibitors and cell cycle checkpoint inhibitors, or a combination of thereof, said method comprising determining tumour type, determining expression levels of PD-L1, determining tumour mutational burden, preparing a DNA damage and repair related genes analysis based on the tumour type and PD-L1 expression levels.

2. A method according to claim 1 wherein the tumour type is selected from bladder, breast, cervical, colorectal, cancer of unknown primary, endometrial, gallbladder, gastric, glioblastoma, glioma, gastro oesophageal junction, head and neck, kidney, liver, lung, melanoma, mesothelioma, oesophageal, ovarian, pancreatic, prostrate, sarcoma, small bowel and thyroid.

3. A method according to either claim 1 or 2 wherein the DNA damage and repair related genes analysis is prepared by using the tumour type and PDL-1 gene expression levels to select the core genes identified in Table A for analysis.

4. A method according to any preceding claim wherein
i) a score of ‘0’ is applied in the absence of PD-L1 expression;
ii) a score of ‘1’ is applied in the presence <7 nLRPM but not 0 in relation to PD-L1 expression;
iii) a score of ‘2’ is applied in the presence 7-10 nLRPM in relation to PD-L1 expression;
iv) a score of ‘3’ is applied in the presence >10 nLRPM in relation to PD-L1 expression;
v) a score of ‘0’ is applied if the tumour mutational burden is ‘low’;
vi) a score of ‘1’ is applied if the tumour mutational burden is ‘high’;
vii) a score of ‘0’ is applied if there are no aberrations in the DNA damage and repair related genes analysis;
viii) a score of ‘1’ is applied if there is 1 aberration in the DNA damage and repair related genes analysis;
xi) a score of ‘2’ is applied if there are 2 aberrations in the DNA damage and repair related genes analysis;
x) a score of ‘3’ is applied if there are aberrations in the DNA damage and repair related genes analysis;
wherein an overall score of 0 is indicative of no susceptibility to the target agent, an overall score of 1-2 indicates a weak response, an overall score of 3-4 indicates a moderate response, and an overall score of 5 to 7 indicates a strong response.

5. A method according to claim 4 wherein the tumour mutational burden is designated ‘low’ if there are <10 mut/MB and the tumour mutational burden is designated ‘high’ if there are ≥1.0 mut/MB.

6. A method according to any preceding claim further comprising administering to a patient found to have a moderate response, an effective amount of the target agent.

7. A method for treating a patient suffering from proliferative disease, said method comprising carrying out a method according to claim 6 using a tumour sample from said patient, developing a customised recommendation for treatment or continued treatment, based upon the overall score, and administering a suitable target agent, therapy or treatment to said patient.

8. A computer or machine-readable cassette programmed to implement the method according to any of the preceding claims.

9. A system for identifying patients suffering from proliferative disease who would respond an agent targeting a cell pathway or components thereof comprising an immune-checkpoint comprising components of the PD1/PD-L1 pathway, an agent targeting DDR/MMR signalling pathway comprising PARP inhibitors, DDR inhibitors and cell cycle checkpoint inhibitors, or a combination of thereof, said system comprising:
a processor; and
a memory that stores code of an algorithm that, when executed by the processor, causes the computer system to:
receive data regarding tumour type of a sample;
receive data regarding level of expression of PD-L1 in the sample;
receive data regarding level of the tumour mutational burden in said sample;
receive data regarding level of DNA damage and repair related genes analysis based on the tumour type and PD-L1 levels;
analyse and transform the input levels via an algorithm to provide an output indicative of the level of susceptibility of said patient to treatment using the target agent;
display the output on a graphical interface of the processor.

10. A system according to claim 9 wherein instead of receiving the data the system, the memory further comprises code which allows at least one of the levels to be determined by the system.

11. A system according to claims 9 and 10 wherein the memory further comprises code to provide a customised recommendation for the treatment of the patient, based upon the output.

12. A system according to claim 11 wherein the customised recommendation is displayed on a graphical interface of the processor.

13. A non-transitory computer-readable medium storing instructions that, when executed by a processor, cause a computer system to identify patients suffering from proliferative disease who would respond to treatment using an agent targeting a cell pathway or components thereof comprising an immune-checkpoint comprising components of the PD1/PD-L1 pathway, an agent targeting DDR signalling pathway comprising PARP inhibitors, DDR inhibitors and cell cycle checkpoint inhibitors, or a combination of thereof, by:
receiving data regarding tumour type of a sample;
receiving data regarding level of expression of PD-L1 in the sample;
receiving data regarding level of the tumour mutational burden in said sample;
receiving data regarding level of DNA damage and repair related genes analysis based on the tumour type and PD-L1 levels;
analysing and transforming the input levels via an algorithm to provide an output indicative of the level of susceptibility of said patient to treatment using the target agent;
displaying the output on a graphical interface of the processor.

14. A non-transitory computer-readable medium according to claim 13 further storing instructions for developing a customised recommendation for treatment of the patient based upon the output and displaying the customized recommendation on a graphical interface of the processor.
